(19) United States
(12) Patent Application Publication
(10) Pub. No.: US 2003/0170838 A1
(43) Pub. Date: Sep. 11, 2003

(54) Novel polynucleotides and polypeptides encoded thereby

(76) Inventors: Vishnu S. Mishra, Gainsville, FL (US); Kimberly Ann Spytek, New Haven, CT (US); Raymond J. Taupier JR., East Haven, CT (US); Corine A. Vernet, L North Branford, CT (US); Steven D. Colman, Guilford, CT (US); Linda Gorman, East Haven, CT (US); Velizar T. Tchernev, Branford, CT (US); Uriel M. Malyankar, North Branford, CT (US); Suresh Shenoy, Branford, CT (US); Muralidhara Padigaru, Branford, CT (US); Valerie L. Gerlach, Branford, CT (US); John R. MacDougall, Hamden, CT (US); Glennda Smithson, Guilford, CT (US); Isabelle Millet, Milford, CT (US); John Peyman, New Haven, CT (US); David Stone, Guilford, CT (US); Erik Gunther, Branford, CT (US); Karen Ellerman, Branford, CT (US); Li Li, Branford, CT (US); Luca Rastelli, Guilford, CT (US); Bryan Zerhusen, Branford, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 09/954,342

(22) Filed: Sep. 17, 2001

Related U.S. Application Data
(60) Provisional application No. 60/233,382, filed on Sep. 18, 2000.
(60) Provisional application No. 60/240,498, filed on Oct. 13, 2000.
(60) Provisional application No. 60/260,284, filed on Jan. 8, 2001.
(60) Provisional application No. 60/260,973, filed on Jan. 11, 2001.
(60) Provisional application No. 60/264,794, filed on Jan. 29, 2001.
(60) Provisional application No. 60/238,398, filed on Oct. 6, 2000.
(60) Provisional application No. 60/232,675, filed on Sep. 15, 2000.
(60) Provisional application No. 60/274,862, filed on Mar. 9, 2001.
(60) Provisional application No. 60/233,801, filed on Sep. 19, 2000.
(60) Provisional application No. 60/232,676, filed on Sep. 15, 2000.
(60) Provisional application No. 60/233,960, filed on Sep. 20, 2000.
(60) Provisional application No. 60/233,402, filed on Sep. 18, 2000.
(60) Provisional application No. 60/233,521, filed on Sep. 19, 2000.
(60) Provisional application No. 60/233,522, filed on Sep. 19, 2000.
(60) Provisional application No. 60/232,679, filed on Sep. 15, 2000.

(51) Int. Cl. C12N 9/00; C07K 14/435; C07H 21/04

(52) U.S. Cl. 435/183; 435/69.1; 435/325; 435/320.1; 530/350; 536/23.2

(57) ABSTRACT

Disclosed herein are nucleic acid sequences that encode Wnt, zinc transporter, mitsugumin29, slit-3, LRR/GPCR, major histocompatability complex enhancer protein MAD3, interleukin 9, 5-hydroxytryptamine receptor, and thioredoxin related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. No. 60/233,382, filed Sep. 18, 2000; U.S. Ser. No. 60/240,498, filed Oct. 13, 2000; U.S. Ser. No. 60/260,284, filed Jan. 8, 2001; U.S. Ser. No. 60/260,973, filed Jan. 11, 2001; U.S. Ser. No. 60/264,794, filed Jan. 29, 2001; U.S. Ser. No. 60/238,398, filed Oct. 6, 2000; U.S. Ser. No. 60/232,675, filed Sep. 15, 2000; U.S. Ser. No. 60/274,862, filed Mar. 9, 2001; U.S. Ser. No. 60/233,801, filed Sep. 19, 2000; U.S. Ser. No. 60/232,676, filed Sep. 15, 2000; U.S. Ser. No. 60/233,960, filed Sep. 20,2000; U.S. Ser. No. 60/233,402, filed Sep. 18, 2000; U.S. Ser. No. 60/233,521, filed Sep. 19, 2000; U.S. Ser. No. 60/233,522, filed Sep. 19, 2000; and U.S. Ser. No. 60/232,679, filed Sep. 15, 2000, each of which is incorporated herein by reference in its entirety.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, orNOV1a, NOV1b, NOV1c, NOV2a, NOV2b, Nov2c, NOV3a, NOV3b, NOV4a, NOV4b, NOV5a, NOV5b, NOV6, NOV7, NOV8, and NOV9 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, and 31. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, bomolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, and 32. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29,and31.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17, 19, 21, 23, 25, 27, 29, and 31) or a complement of said oligonucleotide.

Also included in the invention are substantially purified NOVX polypeptides (SEQ ID NOS:2, 4, 6, 8, 10, 12, 14, 16, 18, 20, 22, 24, 26, 28, 30, and 32). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., developmental disorders, endocrine disorders, vascular disorders, infectious disease, anorexia, cancer, neurodegenerative disorders, lung disorders, reproductive disorders, Alzheimer's Disease, Parkinson's Disease, immune disorders, and hematopoietic disorders, or other disorders related to cell signal processing and metabolic pathway modulation. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from: neurodegenerative diseases (e.g. Alzheimer's disease, Parkinson's disease, Huntington's disease, Multiple Sclerosis, Amyotropic Lateral Sclerosis), acute brain injury (e.g. stroke, head injury, cerebral palsy), CNS dysfunctions (e.g. depression, epilepsy, and schizophrenia), disorders affecting carbohydrate metabolism (e.g. galactosemia and hereditary fructose intolerance), tissue disorders (e.g. Wiskott-Aldrich syndrome, Aldrich syndrome, Eczema-Thrombocytopenia-Immunodeficiency syndrome, thrombocytopenia, night blindness, Batten disease, Ceroid Lipofuscinosis, Rett syndrome and Pick disease), disorders linked to abnormal angiogeniesis (e.g. cancer), asthma, azoospermia, learning disabilities, facial dysmorphism, autoimmune encephalomyelitis, X-linked severe combined immunodeficiency, and other immunological disorders, seizures, migraines, inflammation, autoimmune disorders, and other disorders affecting sleep, appetite, thermoregulation, pain perception, hormone secretion, and sexual behavior.

The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof.

The invention further includes a method for screening for a modulator of disorders or syndromes including, e.g., developmental disorders, endocrine disorders, vascular disorders, infectious disease, anorexia, cancer, neurodegenerative disorders, lung disorders, reproductive disorders, immune and autoimmune disorders, and/or other disorders related to cell signal processing and metabolic pathway modulation. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., developmental disorders, endocrine disorders, vascular disorders, infectious disease, anorexia, cancer, neurodegenerative disorders, lung disorders, reproductive disorders, immune and autoimmune disorders, and/or other disorders related to cell signal processing and metabolic pathway modulation by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., developmental disorders, endocrine disorders, vascular disorders, infectious disease, anorexia, cancer, neurodegenerative disorders, lung disorders, reproductive disorders, immune and autoimmune disorders, and/or other disorders related to cell signal processing and metabolic pathway modulation. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g. a human subject), in an amount sufficient to alleviate or prevent the pathological condition. In preferred embodiments, the disorder, includes, e.g., developmental disorders, endocrine disorders, vascular disorders, infectious disease, anorexia, cancer, neurodegenerative disorders, lung disorders, reproductive disorders, immune and autoimmune disorders, and/or other disorders related to cell signal processing and metabolic pathway modulation.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

Included in the invention are novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to the Wnt gene family. Thus, NOV1 polypeptides of the invention include those that function similarly to members of the Wnt gene family. This gene family encodes a class of cysteine rich proteins that are known to play an important role in vertebrate development and differentiation. Wnt gene family is involved in the signaling pathway that decides the fate of embryonic neural cells that take part in development of the brain. Recent work has shown that Wnt signaling controls initial formation of the neural plate and many subsequent patterning decisions in the embryonic nervous system, including formation of the neural crest. Wnt protein signaling continues to be important at later stages of development. Wnt proteins have been shown to regulate the anatomy of the neuronal cytoskeleton and the differentiation of synapses in the cerebellum. Wnt protein signaling has been demonstrated to regulate apoptosis and may participate in degenerative processes leading to cell death in the aging brain. Lymphocyte enhancer factor-1 (LEF-1) mediated Wnt protein signaling has been shown to participate in B cell development. Recent studies have suggested that the Wnt protein signaling pathway may also play a role in Alzheimer's disease.

The Wnt gene family includes several members. Out of those, Wnt-1 and Wnt-3a, encoded secreted signals are coexpressed at the dorsal midline of the developing neural tube, coincident with dorsal patterning. Each signal is essential for embryonic development, Wnt-1 for midbrain patterning, and Wnt-3a for formation of the paraxial mesoderm. Wnt-3a mutant embryos show defects caudal to the forelimb level; somites are absent, the notochord is disrupted, and the central nervous system has a pronounced dysmorphology. Recent genetic studies have shown that the signalling factor Wnt-3a is required for formation of the hippocampus. In addition, studies have shown that primary axis formation depends on Wnt-3. Apart from development and maintenance of the neural cells, Wnt-1 and Wnt-3 have been discovered as activated oncogenes in mouse mammary tumors. Thus, the NOV1 nucleic acids and polypeptides, antibodies and related compounds according to the invention are useful in therapeutic applications in various neurological disorders such as, but not limited to, neurodegenerative diseases (e.g. Alzheimer's, Parkinson's, Multiple Sclerosis, Huntington's, Amyotropic Lateral Sclerosis), acute brain injury (e.g. stroke, head injury, cerebral palsy) and a large number of CNS dysfunctions (e.g. depression, epilepsy, and schizophrenia).

NOV2 is homologous to the Zinc-transporter-like (ZNT) family of proteins. Thus, NOV2 polypeptides of the present invention include those that function similarly to members of the ZNT family. Zinc transporters play a role in transporting zinc ions into cells, and regulating processes such as cell survival and proliferation. Zinc-binding proteins have been identified in the brain and regulate the steady state concentration of zinc. Because zinc is a potent inhibitor of numerous sulphydryl-containing enzymes, zinc-binding proteins may plat a role in preventing Central Nervous System toxicity by preventing the rise of free zinc in the brain. Apart from maintenance of neural cells, zinc-binding proteins have been found to play an important role in carbohydrate metabolism. The NOV2 nucleic acids and poly peptides, antibodies and related compounds according to the invention, therefore, are useful in therapeutic applications in neurological maintenance and various disorders in carbohydrate metabolism such as Galactosemia and Hereditary Fructose Intolerance.

NOV3 is homologous to the Mitsugumin29-like (MG29) family of proteins, which is a member of the synaptophysin family. Thus, NOV3 polypeptides of the invention include those that function similarly to MG29 and other members of the synaptophysin family. Synaptophysin and synaptoporin are related glycoproteins: they are the major integral membrane proteins of a certain class of small neurosecretory vesicles, although they may also be found in vesicles of various non-endocrine cells. The polypeptide chain spans the membrane four times and possibly acts as an ion or solute channel. Recently MG29 unique to the triad junction in skeletal muscle was identified as a novel member of the synaptophysin family; the members of this family have four transmembrane segments and are distributed on intracellular vesicles. Mouse MG29 cDNA and genomic DNA containing the gene has been isolated and analyzed. The MG29 gene mapped to the mouse chromosome 3 F3-H2 is closely related to the synaptophysin gene in exon-intron organization, which indicates their intimate relationship in molecular evolution. RNA blot hybridization and immunoblot analysis revealed that MG29 is expressed abundantly in skeletal muscle and at lower levels in the kidney. Immunofluorescence microscopy demonstrated that MG29 exists specifically in cytoplasmic regions of the proximal and distal tubule cells in the kidney. The results obtained suggest that MG29 is involved in the formation of specialized endoplasmic reticulum systems in skeletal muscle and renal tubule cells.

Physiological roles of the members of the synaptophysin family, carrying four transmembrane segments and being basically distributed on intracellular membranes including synaptic vesicles, have not been established yet. Recently, MG29 was identified as a novel member of the synaptophysin family from skeletal muscle. MG29 is expressed in the junctional membrane complex between the cell surface transverse (T) tubule and the sarcoplasmic reticulum (SR), called the triad junction, where the depolarization signal is converted to Ca(2+) release from the SR. The distribution and protein structure of MG29 suggests that this protein is involved in communication between the T-tubular and junctional SR membranes. Further, the morphological and functional abnormalities of the mutant muscle seem to be related to each other and indicate that MG29 is essential for both refinement of the membrane structures and effective excitation-contraction coupling in the skeletal muscle triad junction.

The NOV3 nucleic acids and poly peptides, antibodies and related compounds according to the invention, therefore, are useful in therapeutic applications in tissue disorders such as, but not limited to, Wiskott-Aldrich syndrome, Aldrich syndrome, Eczema-Thrombocytopenia-Immunodeficiency syndrome, Thrombocytopenia, Night Blindness, Amyotropic lateral sclerosis, Batten disease, Ceroid Lipofuscinosis, Rett syndrome and Pick disease (lobar atrophy).

NOV4 is homologous to the Slit-3-like family of proteins. Thus, NOV4 polypeptides of the invention include those that function similarly to Slit-3 and members of the Slit family of proteins. Slit is expressed in the midline of the central nervous system both in vertebrates and invertebrates. Each Slit gene encodes a putative secreted protein, which contains conserved protein-protein interaction domains including leucine-rich repeats (LRR) and epidermal growth factor (EGF)-like motifs, like those of the Drosophila protein. Northern blot analysis has revealed that the human Slit-1, -2, and -3 mRNAs are exclusively expressed in the brain, spinal cord, and thyroid, respectively. Slit proteins may participate in the formation and maintenance of the nervous and endocrine systems by protein-protein interactions. NOV4 nucleic acids and polypeptides, antibodies and related compounds according to the invention, therefore, are useful in therapeutic applications in various neurological disorders such as, but not limited to, neurodegenerative diseases (e.g. Alzheimer's, Parkinson's, Multiple Sclerosis, Huntington's, Amyotropic Lateral Sclerosis), acute brain injury (e.g. stroke, head injury, cerebral palsy) and a large number of CNS dysfunctions (e.g. depression, epilepsy, and schizophrenia).

NOV5 is homologous to the Leucine Rich Repeat (LRR)/GPCR family of proteins. Thus, NOV5 polypeptides of the invention include those that function similarly to other members of the Leucine Rich Repeat (LRR)/GPCR family. Proteins within this family have been implicated in tissue organization, collagen fibril orienting and ordering during ontogeny, and in pathological processes such as wound healing, tissue repair, and tumor stroma formation. Thus, NOV5 will have important structural and/or physiological functions characteristic of tumor angiogenisis. Specifically, NOV5 will be involved in the remodeling of the extracellular matrix that occurs during tumor angiogenesis as suggested by the presence of a LRR domain in the LRR/GPCR-like protein. NOV5 polypeptide will also act as a receptor for an unknown ligand and mediate downstream signalling.

The NOV5 nucleic acids and polypeptides, antibodies and related compounds according to the invention are useful, therfore, in potential diagnostic and therapeutic applications implicated in various diseases and disorders described below and/or other pathologies. For example, the compositions of NOV5 will have efficacy for treatment of patients suffering from disorders linked to abnormal angiogenesis, like cancer and more specifically aggressive, metastatic cancer, in particular tumors of the lung, kidney, brain, liver and colon.

NOV6 is homologous to the Major Histocompatibility Complex Enhancer-Binding Protein, MAD3. Thus, NOV6 polypeptides of the invention include those that function similarly to MAD3 and other members of the MAD family of proteins. MAD3 is a checkpoint protein required for cell cycle arrest in response to loss of microtubule function. The protein contains 5 ank repeats and is induced in adherent monocytes. MAD3 may regulate transcriptional responses to NF-KAPPA-B, including adhesion- dependent pathways of monocyte activation. It interacts directly with the nf-kappa-b complex, presumably through the P65 subunit.

The NOV6 nucleic acids and polypeptides, antibodies and related compounds according to the invention, therefore, are useful in potential diagnostic and therapeutic applications implicated in various diseases and disorders described below and/or other pathologies. For example, the compositions of NOV6 will have efficacy for treatment of patients suffering from disorders linked to abnormal angiogenesis, like cancer and more specifically aggressive, metastatic cancer, in particular tumors of the lung, kidney, brain, liver and colon.

NOV7 is homologous to the Interleukin-9 protein. Thus, NOV7 polypeptides of the invention include those that function similarly to Interleukin-9. Interleukin-9 (IL-9) is a cytokine that supports IL-2 independent and IL-4 independent growth of helper T-cells. Interleukin-9 is a cytokine that serves as a regulator of both lymphoid and myeloid systems. IL-9 may play a role in Hodgkin disease and large cell anaplastic lymphoma as an autocrine growth factor.

The NOV7 nucleic acids and polypeptides, antibodies and related compounds according to the invention, therefore, are useful in potential diagnostic and therapeutic applications implicated in various diseases and disorders described below and/or other pathologies. For example, the compositions of the present invention will have efficacy for treatment of patients suffering from asthma, various types of cancer, azoospermia, learning disabilities, facial dysmorphism, multiple sclerosis, autoimmune encephalomyelitis, X-linked severe combined immunodeficiency and other immunological disorders.

NOV8 is homologous to the hydroxytryptamine receptor-like family of proteins. Thus, NOV8 polypeptides of the invention include those that function similarly to the hydroxytryptamine receptor family. The neurotransmitter serotonin (5-hydroxytryptamine; 5-HT) exerts a wide variety of physiologic functions through a multiplicity of receptors and may be involved in human neuropsychiatric disorders such as anxiety, depression, or migraine. These receptors consist of 4 main groups, 5-HT-1, 5-HT-2, 5-HT-3, and 5-HT4, subdivided into several distinct subtypes on the basis of their pharmacologic characteristics, coupling to intracellular second messengers, and distribution within the nervous system. The serotonergic receptors belong to the multi 5-Hydroxytryptamine Receptor family of receptors coupled to guanine nucleotide-binding proteins. Thus, these receptors can modulate the activity of neural reward pathways and therefore the effects of various drugs of abuse.

The NOV8 nucleic acids and polypeptides, antibodies and related compounds according to the invention, therefore, are useful in potential diagnostic and therapeutic applications implicated in various diseases and disorders described below and/or other pathologies. For example, the compositions of the present invention will have efficacy for treatment of patients suffering from seizures, Alzheimer's disease, mental depression, migraines, epilepsy, obsessive-compulsive behavior (schizophrenia), and other disorders affecting sleep, appetite, thermoregulation, pain perception, hormone secretion, and sexual behavior.

NOV9 is homologous to a thioredoxin-like family of proteins. Thioredoxin is involved in several cellular processes such as protein assembly and repair, resistance to ionizing radiation, DNA replication, transcription, and cell division. In the NADP/thioredoxin system, the reduction of thioredoxin is linked to NADPH via a flavin enzyme, NADP-thioredoxin reductase(NTR). Thus, the NOV9 nucleic acids, polypeptides, antibodies and related compounds according to the invention are useful in therapeutic and diagnostic applications implicated in, for example, inflammation, autoimmune disorders, aging and cancer, and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

A NOV1 polypeptide according to the invention includes a Wnt-like protein. The NOV1 nucleic acid sequences disclosed herein map to chromosome 1. The nucleic acid sequence (and encoded polypeptide) of three NOV1 sequences-NOV1a, NOV1b, and NOV1c are provided.

NOV1a

A NOV1a (alternatively referred to herein as sggc_draft_dj881p19_{—}20000725, sggc_draft_dj881p19_{—}20000725-A, X56842_da1, or CG55702-01), includes the 1082 nucleotide sequence (SEQ ID NO:1) and which encodes a Wnt-like protein with the amino acid sequence shown in Table 1A. The disclosed ORF begins with a Kozak consensus ATG initiation codon at nucleotides 16-18 and ends with a TAG codon at nucleotides 1072-1074. Untranslated regions upstream from the initiation codon and downstream from the termination codon are underlined in Table 1A, and the start and stop codons are in bold letters.

TABLE 1A
NOV1a Nucleotide Sequence (SEQ ID NO:1)

CCCTCTCGCGCGGCGATGGCCCCACTCGGATACTTCTTACTCCTCTGCAGCCTGAAGCAGGCTCTGG
GCAGCTACCCGATCTGGTGGTCGCTGGCTGTTGGGCCACAGTATTCCTCCCTGGGCTCGCAGCCCAT
CCTGTGTGCCAGCATCCCGGGCCTGGTCCCCAAGCAGCTCCGCTTCTGCAGGACTACGTGGAGATC
ATGCCCAGCGTGGCCGAGGGCATCAAGATTGGCATCCAGGAGTGCCAGCACCAGTTCCGCGGCCGCC
GGTGGAACTGCACCACCGTCCACGACAGCCTGGCCATCTTCGGGCCCGTGCTGGACAAAGCTACCAG
GGAGTCGGCCTTTGTCCACGCCATTGCCTCAGCCGGTGTGGCCTTTGCAGTGACACGCTCATGTGCA
GAAGGCACGGCCGCCATCTGTGGCTGCAGCAGCCGCCACCAGGGCTCACCAGGCAAGGGCTGGAGT
GGGGTGGCTGTAGCGAGGACATCGAGTTTGGTGGGATGGTGTCTCGGGAGTTCGCCGACGCCCGGGA
GAACCGGCCAGATGCCCGCTCAGCCATGAACCGCCACAACAACGAGGCTGGGCGCCAGGCCATCGCC
AGCCACATGCACCTCAAGTGCAAGTGCCACGGGCTGTCGGGCAGCTGCGAGGTGAAGACATGCTGGT
GGTCGCAACCCGACTTCCGCGCCATCGGTGACTTCCTCAAGGACAAGTACGACAGCGCCTCGGAGAT
GGTGGTGGAGAAGCACCGGGAGTCCCGCGGCTGGGTGGAGACCCTGCGGCCGCGCTACACCTACTTC
AAGGTGCCCACGGAGCGCGACCTGGTCTACTACGAGGCCTCGCCCAACTTCTGCGAGCCCAACCCTG
AGACGGGCTCCTTCGGCACGCGCGACCGCACCTGCAACGTCAGCTCGCACGGCATCGACGGCTGCGA
CCTGCTGTGCTGCGGCCGCGGCCACAACGCGCGAGCGGAGCGGCGCCGGGAGAAGTGCCGCTGCGTG
TTCCACTGGTGCTGCTACGTCAGCTGCCAGGAGTGCACGCGCGTCTACGACGTGCACACCTGCAAGT
AGGCACCGGC

Variant sequences of NOV1b are included in Example 2, Table 48 and 49. A variant sequence can include a single nucleotide polymorphism (SNP). A SNP can, in some instances, be referred to as a “cSNP” to denote that the nucleotide sequence containing the SNP originates as a cDNA.

The NOV1a polypeptide (SEQ ID NO:2) encoded by SEQ ID NO:1 is 352 amino acid residues in length, has a molecular weight of 39364.3 Daltons, and is presented in Table 1B.

TABLE 1B
NOV1a protein sequence (SEQ ID NO:2)

MAPLGYFLLLCSLKQALGSYPIWWSLAVGPQYSSLGSQPILCASIPGLVPKQLRFCRNYVEIMPSVA
EGIKIGIQECQHQFRGRRWNCTTVHDSLAIFGPVLDKATRESAFVHAIASAGVAFAVTRSCAEGTAA
ICGCSSRHQGSPGKGWKWGGCSEDIEFGGMVSREFADARENRPDARSAMNRHNNEAGRQAIASHMHL
KCKCHGLSGSCEVKTCWWSQPDFRAIGDFLKDKYDSASEMVVEKHRESRGWVETLRPRYTYFKVPTE
RDLVYYEASPNFCEPNPETGSFGTRDRTCNVSSHGIDGCDLLCCGRGHNARAERRREKCRCVFHWCC
YVSCQECTRVYDVHTCK

NOV1b

A NOV1 variant also includes a NOV1b (alternatively referred to herein as GM_AL136379_A). A disclosed NOV1b sequence of 1116 nucleotide sequence (SEQ ID NO:3) is shown in Table 1C. The disclosed ORF begins with a Kozak consensus ATG initiation codon at nucleotides 31-33 and ends with a TAG codon at nucleotides 1087-1089. Untranslated regions upstream from the initiation codon and downstream from the termination codon are underlined in Table 1C, and the start and stop codons are in bold letters.

TABLE 1C
NOV1b Nucleotide Sequence (SEQ ID NO:3)

TCCCGGCCCTCCGCGCCCTCTCGCGCGGCATGGCCCCACTCGGATACTTCTTACTCCTCTGCAGCC
TGAAGCAGGCTCTGGGCAGCTACCCGATCTGGTGGTCGCTGGCTGTTGGGCCACAGTATTCCTCCCT
GGGCTCGCAGCCCATCCTGTGTGCCAGCATCCCGGGCCTGGTCCCCAAGCAGCTCCGCTTCTGCAGG
AACTACGTGGAGATCATGCCCAGCGTGGCCGAGGGCATCAAGATTGGCATCCAGGAGTGCCAGCACC
AGTTCCGCGGCCGCCGGTGGAACTGCACCACCGTCCACGACAGCCTGGCCATCTTCGGGCCCGTGCT
GGACAAAGCTACCAGGGAGTCGGCCTTTGTCCACGCCATTGCCTCAGCCGGTGTGGCCTTTGCAGTG
ACACGCTCATGTGCAGAAGGCACGGCCGCCATCTGTGGCTGCAGCAGCCGCCACCAGGGCTCACCAG
GCAAGGGCTGGAAGTGGGGTGGCTGTAGCGAGGACATCGAGTTTGGTGGGATGGTGTCTCGGGAGTT
CGCCGACGCCCGGGAGAACCGGCCAGATGCCCGCTCAGCCATGAACCGCCACAACAACGAGGCTGGG
CGCCAGGCCATCGCCAGCCACATGCACCTCAAGTGCAAGTGCCACGGGCTGTCGGGCAGCTGCGAGG
TGAAGACATGCTGGTGGTCGCAACCCGACTTCCGCGCCATCGGTGACTTCTTCAAGGACAAGTACGA
CAGCGCCTCGGAGATGGTGGTGGAGAAGCACCGGGAGTCCCGCGGCTGGGTGGAGACCCTGCGGCCG
CGCTACACCTACTTCAAGGTGCCCACGGAGCGCGACCTGGTCTACTACGAGGCCTCGCCCAACTTCT
GCGAGCCCAACCCTGAGACGGGCTCCTTCGGCACGCGCGACCGCACCTGCAACGTCAGCTCGCACGG
CATCGACGGCTGCGACCTGCTGTGCTGCGGCCGCGGCCACAACGCGCGAGCGGAGCGAGCGCCGGAG
AAGTGCCGCTGCGTGTTCCACTGGTGCTGCTACGTCAGCTGCCAGGAGTGCACGCGCGTCTACGACG
TGCACACCTGCAAGTAGGCACCGGCCGCGGCTCCCCCTGGACGG

Variant sequences of NOV1b are included in Example 2, Table 50. A variant sequence can include a single nucleotide polymorphism (SNP). A SNP can, in some instances, be referred to as a “cSNP” to denote that the nucleotide sequence containing the SNP originates as a cDNA.

The NOV1b protein (SEQ ID NO:4) encoded by SEQ ID NO:3 is 352 amino acid residues in length, has a molecular weight of 39364.3 Daltons, and is presented in Table 1D.

TABLE 1D
NOV1b protein sequence (SEQ ID NO:4)

MAPLGYFLLLCSLKQALGSYPIWWSLAVGPQYSSLGSQPILCASIPGLVPKQLRFCRNYVEIMPSVA
EGIKIGIQECQHQFRGRRWNCTTVHDSLAIFGPVLDKATRESAFVHAIASAGVAFAVTRSCAEGTAA
ICGCSSRHQGSPGKGWKWGGCSEDIEFGGMVSREFADARENRPDARSAMNRHNNEAGRQAIASHMHL
KCKCHGLSGSCEVKTCWWSQPDFRAIGDFLKDKYDSASEMVVEKHRESRGWVETLRPRYTYFKVPTE
RDLVYYEASPNFCEPNPETGSFGTRDRTCNVSSHGIDGCDLLCCGRGHNARAERRREKCRCVFHWCC
YVSCQECTRVYDVHTCK

NOV1c

A NOV1 variant is a NOV1c (alternatively referred to herein as CG55702-04) disclosed, includes the 947 nucleotide sequence (SEQ ID NO:5) shown in Table 1E. The NOV1c ORF begins at nucleotides 5-7 and ends at nucleotides 944-946. Untranslated regions upstream from the initiation codon and downstream from the termination codon are underlined in Table 1E, and the start and stop codons are in bold letters.

TABLE 1E
NOV1c Nucleotide Sequence (SEQ ID NO:5)

CCTACTTGCAGGTGTGCACGTCGTAGACGCGCGTGCACTCCTGGCAGCTGACGTAGCAGCACCAGTG
GAACACGCAGCGGCACTTCTCCCGGCGCCGCTCCGCTCGCGCGTTGTGGCCGCGGCCGCAGCACAGC
AGGTCGCAGCCGTCGATGCCGTGCGAGCTGACGTTGCAGGTGCGGTCGCGCGTGCCGAAGGAGCCCG
TCTCAGGGTTGGGCTCGCAGAAGTTGGGCGAGGCCTCGTAGTAGACCAGGTCGCGCTCCGTGGGCAC
CTTGAAGTAGGTGTAGCGCGGCCGCAGGGTCTCCACCCAGCCGCGGGACTCCCGGTGCTTCTCCACC
ACCATCTCCGAGGCGCTGTCGTACTTGTCCTGGCGCCCAGCCTCGTTGTTGTGGCGGTTCATGGCTG
AGCGGACATCTGGCCGGTTCTCCCGGGCGTCGGCGAACTCCCGAGACACCATCCCACCAAACTCGAT
GTCCTCGCTACAGCCACCCCACTTCCAGCCCTTGCCTGGTGAGCCCTGGTGGCGGCTGCTGCAGCCA
CAGATGGCGGCCGCGCCTTCTGCACATGAGCGTGTCACTGCAAAGGCCACACCGGCTGAGGCAATGG
CGTGGACAAAGGCCGACTCCCTGGTAGCTTTGTCCAGCACGGGCCCGAAGATGGCCAGGCTGTCGTG
GACGGTGGTGCAGTTCCACCGGCGGCCGCGGAACTGGTGCTGGCACTCCTGGATGCCGATCTTGATG
CCCTCGGCCACGCTGGGCATGATCTCCACGTAGTTCCTGCAGAAGCGGAGCTGCTTGGGGACCAGGC
CCGGGATGCTGGCACACAGGATGGGCTGCGAGCCCAGGGAGGAATACTGTGGCCCAACAGCCAGCGA
CCACCAGATCGGGTAGCTGCCCAGAGCCTGCTTCAGGCTGCAGAGGAGTAAGGAAGTATCCGAGTGGG
GCCATCAAG

The NOV1c protein (SEQ ID NO:6) encoded by SEQ ID NO:5 is 313 amino acid residues in length, has a molecular weight of 34988.3 Daltons, and is presented in Table 1F.

TABLE 1F
NOV1c protein sequence (SEQ ID NO:6)

MAPLGYFLLLCSLKQALGSYPIWWSLAVGPQYSSLGSQPILCASIPGLVPKQLRFCRNYVEIMPSVA
EGIKIGIQECQHQFRGRRWNCTTVHDSLAIFGPVLDKATRESAFVHAIASAGVAFAVTRSCAEGAAA
ICGCSSRHQGSPGKGWKWGGCSEDIEFGGMVSREFADARENRPDVRSAMNRHNNEAGRQDKYDSASE
MVVEKHRESRGWVETLRPRYTYFKVPTERDLVYYEASPNFCEPNPETGSFGTRDRTCNVSSHGIDGC
DLLCCGRGHNARAERRREKCRCVFHWCCYVSCQECTRVYDVHTCK

A Nov1c polypeptide may vary from the disclosed amino acid sequence at the N-terminus and/or at the C-terminus by one amino acid residue. Specifically, a NOV1c polypeptide is disclosed wherein a leucine residue precedes the N-terminal methionine residue. Alternatively, a NOV1c polypeptide is disclosed wherein a leucine precedes the N-terminal methionine residue and the C-terminus is extended by one amino acid residue selected from one of the 20 naturally occurring amino acids. In yet another form, NOV1c polypeptide has an N-terminal methionine residue and the C-terminus is extended by one amino acid residue selected from one of the 20 naturally occurring amino acids.

NOV1 Clones

The Psort profile for NOV1 predicts that this polypeptide sequence is likely to be localized outside the cell with a certainty of 0.4037. The Signal P predicts a likely cleavage site for a NOV1 polypeptide is between positions 18 and 19, i.e., at the dash in the sequence ALG-SY.

A search against the Patp database, a proprietary database that contains sequences published in patents and patent publications, yielded several homologous proteins. These proteins are identified in Table 1G.

TABLE 1G
Patp results for NOV 1

| | | Smallest |
|---|---|---|
| | | Sum |
| | High | Prob |
| Sequences producing High-scoring Segment Pairs: | Score | P(N) |
| >patp:AAY57596 Murine Wnt-3a protein | 1892 | 2.9e−195 |
| >patp:AAW30618 Human Wnt-3 protein | 1704 | 2.4e−175 |
| >patp:AAY41719 Human PR0864 protein | 902 | 2.3e−90 |

In a BLAST search of public sequence databases, it was found, for example, that the nucleic acid sequence of NOV1a has 939 of 1075 bases (87%) identical to a Wnt-3A cysteine-rich protein mRNA from Mus musculus (GENBANK-ID: MMWNT3A|acc:X56842 ). The full amino acid sequence of the protein of the invention was found to have 338 of 352 amino acid residues (96%) identical to, and 344 of 352 amino acid residues (97%) similar to the 352 amino acid residue Wnt-3A PROTEIN PRECURSOR from Mus musculus (SWISSPROT-ACC:P27467).

Similarly, in a BLAST search of public sequence databases, it was found, for example, that the nucleic acid sequence of NOV1b has 946 of 1084 bases (87%) identical to a Wnt-3A mRNA from Mus musculus (GENBANK-ID: X56842). The full amino acid sequence of the protein of NOV1b was found to have 338 of 352 amino acid residues (96%) identical to, and 344 of 352 amino acid residues (97%) similar to, the Wnt-3A protein from Mus musculus (ACC:P27467). Furthermore, in a BLAST search of public sequence databases, it was found, for example, that the full amino acid sequence of the protein of NOV1c was found to have 191 of 193 amino acid residues (98%) identical to human Wnt-3A (TREMBLNEW-ACC:BAB61052).

Additional BLAST results are shown in Table 1H. In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the IIT BLAST analysis, matched the Query IIT sequence purely by chance is the E value. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences. Blasting is performed against public nucleotide databases such as GenBank databases and the GeneSeq patent database. For example, BLASTX searching is performed against public protein databases, which include GenBank databases, SwissProt, PDB and PIR.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

TABLE 1H
BLAST results for NOV1

| Gene Index/ | | Length | Identity | Positives | |
|---|---|---|---|---|---|
| Identifier | Protein/Organism | (aa) | (%) | (%) | Expect |
| ptnr:TREMBLNEW- | WNT3A | 352 | 352/352 | 352/352 | 1.7 e − |
| ACC:BAB61052 | [Homo sapiens] | | (100%) | (100%) | 202 |
| ptnr:SWISSPROT- | WNT-3A PROTEIN | 352 | 338/352 | 344/352 | 4.6 e − |
| ACC:P27467 | PRECURSOR | | (96%) | (97%) | 195 |
| | [Mus musculus] | | | | |
| ptnr:SWISSPROT- | WNT-3A PROTEIN | 352 | 296/352 | 321/352 | 5.4 e − |
| ACC:P31285 | PRECURSOR | | (84%) | (91%) | 176 |
| | (XWNT-3A) | | | | |
| | [Xenopus laevis] | | | | |
| ptnr:SWISSNEW- | WNT-3 proto-oncogene | 355 | 297/350 | 319/350 | 3.8 e − |
| ACC:P56703 | protein precursor - | | (84%) | (91%) | 175 |
| | [Homo sapiens] | | | | |

A multiple sequence alignment is given in Table 1I, disclosed NOV1 protein sequences are shown on line 1, in a ClustalW analysis comparing NOV1 with related protein sequences is disclosed in Table 1H. The homologies shared by NOV1a, NOV 1b, and NOV1c polypeptides are also shown in Table 1I.

TABLE 1I
Information for the ClustalW proteins:

1. >NOV1a; SEQ ID NO:2
2. >NOV1b; SEQ ID NO:4
3. >NOV1c; SEQ ID NO:6
4. >BAB61052/WNT3A [Homo sapiens]; SEQ ID NO:33
5. >P27467/WNT-3A protein precursor [Mus musculus]; SEQ ID NO:34
6. >P31285/WNT-3A protein precursor [Xenopus laevis; SEQ ID NO:35
7. >P56703/WNT-3 proto-oncogene protein-precursor [Homo sapiens]; SEQ ID NO:36

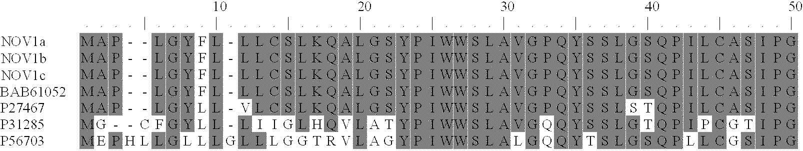

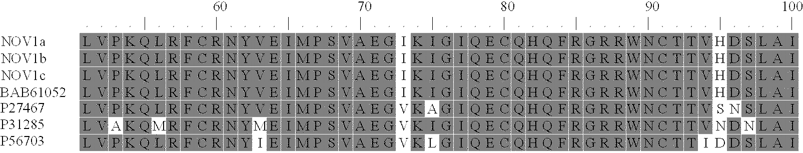

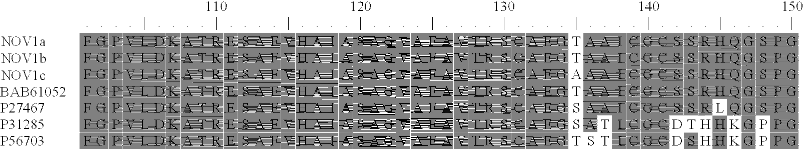

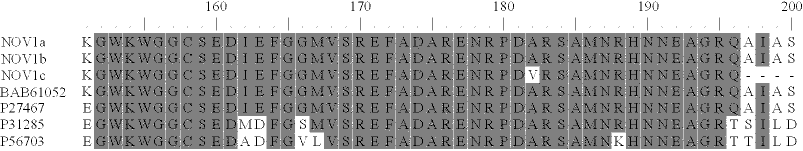

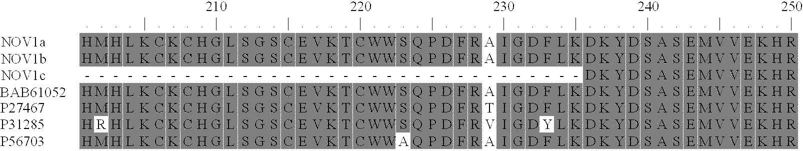

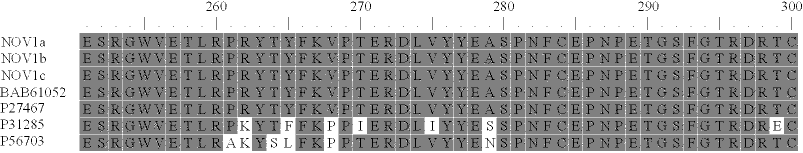

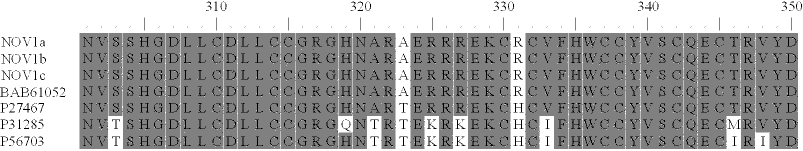

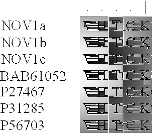

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 4J lists the domain description from DOMAIN analysis results against NOV1.

TABLE 1J
Domain Analysis of NOV 1

| | Region of | | |
|---|---|---|---|
| Model | Homology | Score (bits) | E value |
| Wnt | 41-352 | 742.7 | 8.7e−270 |

The presence of protein regions on NOV1 that are homologous to the Wnt domain (IPR000970) is consistent with the organization of members of the Wnt Protein Family. This indicates that the NOV1 sequence has properties similar to those of other Wnt-like proteins known to contain these domains.

A Wnt-like protein in the invention includes NOV1 sequences expressed in the fetal and adult brain. The expression pattern, map location, domain analysis, and protein similarity information for the invention reveals that the invention includes NOV1 polypeptides that function as a Wnt-like proteins. The NOV1 nucleic acids and proteins of the invention, therefore, are useful in potential therapeutic applications implicated, for example but not limited to, in various pathologies/disorders as described below and/or other pathologies/disorders. Potential therapeutic uses for the invention(s) are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in potential diagnostic and therapeutic applications implicated in various diseases and disorders described below and/or other pathologies. By way of non-limiting example, the compositions of the present invention will have efficacy for treatment of patients suffering from neurological disorders such as neural developmental defects, neurodegenerative diseases (including Alzheimer's disease), cancer (including mammary tumors) and B cell proliferation disorders. It will also be useful for treating disorders in other organs where it is expressed. It can also be used to treat conditions where development and differentiation are impaired and which may be corrected by Wnt-3a signaling pathway. For example, but not limited to, a cDNA encoding the Wnt-like protein may be useful in gene therapy, and the Wnt-like protein may be useful when administered to a subject in need thereof. NOV1 proteins and nucleic acids, or fragments thereof, are useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. The disclosed NOV1 protein has multiple hydrophilic regions, each of which can be used as an immunogen. In one embodiment, a contemplated NOV1 epitope is from about amino acids 50 to 100. In another embodiment, a NOV1 epitope is from about amino acids 120 to 200. In additional embodiments, NOV1 epitopes are from about amino acids 205 to 300, and from about amino acids 301 to 345.

NOV2

A protein of the invention, referred to herein as NOV2, is a Zinc transporter-like protein (ZNT)-like protein. The nucleic acid sequence (and encoded polypeptide) of three NOV2 sequences- NOV2a, NOV2b, and NOV2c are provided.

NOV2a

A NOV2a (alternatively referred to herein as 30370359_da1), includes the 1431 nucleotide sequence (SEQ ID NO:7) shown in Table 2A. The disclosed ORF begins with a Kozak consensus ATG initiation codon at nucleotides 292-294 and ends with a TAG codon at nucleotides 1399-1401. Untranslated regions upstream from the initiation codon and downstream from the termination codon are underlined in Table 2A, and the start and stop codons are in bold letters.

TABLE 2A
NOV2 Nucleotide Sequence (SEQ ID NO:7)

CAGATATCATATGAAAGACATACACACTTCATGTAATGCTACCTGCAAGTCTCCCTAGAAAAGCAGT
TTTTGTAGGTGAAAACAATGAAGCCAGGTAATATTGCAAGGAGGCTGTAATTTTAGCAGACCTACCA
ACAACACTGATGTAGGAAGCTCATTATTTTAATTTCTGGAGCCTTTTAATTTTTTCTTTAGAAAGTG
TATAAATAATTGCAGTGCTGCTTTGCTTCCAAAACTGGGCAGTGAGTTCAACAACAACGACAACAAC
AGCCGCAGCTCATCCTGGCCGTCATGGAGTTTCTTGAAAGAACGTATCTTGTGAATGATAAAGCTGC
CAAGATGTATGCTTTCACACTAGAAAGTGTGGAACTCCAACAGAAACCGGTGAATAAAGATCAGTGT
CCCAGAGAGAGACCAGAGGAGCTGGAGTCAGGAGGCATGTACCACTGCCACAGTGGCTCCAAGCCCA
CAGAAAAGGGGGCGAATGAGTACGCCTATGCCAAGTGGAAACTCTGTTCTGCTTCAGCAATATGCTT
CATTTTCATGATTGCAGAGGTCGTGGGTGGGCACATTGCTGGGAGTCTTGCTGTTGTCACAGATGCT
GCCCACCTCTTAATTGACCTGACCAGTTTCCTGCTCAGTCTCTTCTCCCTGTGGTTGTCATCGAAGC
CTCCCTCTAAGCGGCTGACATTTGGATGGCACCGAGCAGAGATCCTTGGTGCCCTGCTCTCCATCCT
GTGCATCTGGGTGGTGACTGGCGTGCTAGTGTACCTGGCATGTGAGCGCCTGCTGTATCCTGATTAC
CAGATCCAGGCGACTGTGATGATCATCGTTTCCAGCTGCGCAGTGGCGGCCAACATTGTACTAACTG
TGGTTTTGCACCAGAGATGCCTTGGCCACAATCACAAGGAAGTACAAGCCAATGCCAGCGTCAGAGC
TGCTTTTGTGCATGCCCTTGGAGATCTATTTCAGAGTATCAGTGTGCTAATTAGTGCACTTATTATC
TACTTTAAGCCAGAGTATAAAATAGCCGACCCAATCTGCACATTCATCTTTTCCATCCTGGTCTTGG
CCAGCACCATCACTATCTTAAAGGACTTCTCCATCTTACTCATGGAAGGTGTGCCAAAGAGCCTGAA
TTACAGTGGTGTGAAAGAGCTTATTTTAGCAGTCGACGGGGTGCTGTCTGTGCACAGCCTGCACATC
TGGTCTCTAACAATGAATCAAGTAATTCTCTCAGCTCATGTTGCTACAGCAGCCAGCTGGGACAGCC
AAGTGGTTCGGAGAGAATTGCTAAAGCCCTTAGCAAAAGCTTTACGATGCACTCACTCACCATTCA
GATGGAATCTCCAGTTGACCAGGACCCCGACTGCCTTTTCTGTGAAGACCCCTGTGACTAGCTCAGT
CACACCGTCAGTTTCCCAAATTTG

The NOV2a polypeptide (SEQ ID NO:8) encoded by SEQ ID NO:7 is 369 amino acid residues in length, has a molecular weight of 40784.1 Daltons, and is presented using the one-letter amino acid code in Table 2B.

TABLE 2B
NOV2a protein sequence (SEQ ID NO:8)

MEFLERTYLVNDKAAKMYAFTLESVELQQKPVNKDQCPRERPEELESGGMYHCHSGSKPTEKGANEY
AYAKWKLCSASAICFIFMIAEVVGGHIAGSLAVVTDAAHLLIDLTSFLLSLFSLWLSSKPPSKRLTF
GWHRAEILGALLSILCIWVVTGVLVYLACERLLYPDYQIQATVMIIVSSCAVAANIVLTVVLHQRCL
GHNHKEVQANASVRAAFVHALGDLFQSISVLISALIIYFKPEYKIADPICTFIFSILVLASTITILK
DFSILLMEGVPKSLNYSGVKELILAVDGVLSVHSLHIWSLTMNQVILSAHVATAASWDSQVVRREIA
KALSKSFTMHSLTIQMESPVDQDPDCLFCEDPCD

NOV2b

A NOV2b (alternatively referred to herein as CG57799-01), includes the 1623 nucleotide sequence (SEQ ID NO:9) shown in Table 2C. The disclosed ORF begins with a Kozak consensus ATG initiation codon at nucleotides 292-294 and ends with a TAG codon at nucleotides 1558-1560. Untranslated regions upstream from the initiation codon and downstream from the termination codon are underlined in Table 2C, and the start and stop codons are in bold letters.

TABLE 2C
NOV2b Nucleotide Sequence (SEQ ID NO:9)

CAGATATCATATGAAAGACATACACACTTCATGTAATGCTACCTGCAAGTCTCCCTAGAAAGCAGT
TTTTGTAGGTGAAAACAATGAAGCCAGGTAATATTGCAAGGAGGCTGTAATTTTAGCAGACCTACCA
ACAACACTGATGTAGGAAGCTCATTATTTTAATTTCTGGAGCCTTTTAATTTTTTCTTTAGAAAGTG
TATAAATAATTGCAGTGCTGCTTTGCTTCCAAAACTGGGCAGTGAGTTCAACAACAACGACAACAAC
AGCCGCAGCTCATCCTGGCCGTCATGGAGTTTCTTGAAAGAACGTATCTTGTGAATGATAAAGCTGC
CAAGATGTATGCTTTCACACTAGAAAGTGTGGAACTCCAACAGAAACCGGTGAATAAAGATCAGTGT
CCCAGAGAGAGACCAGAGGAGCTGGAGTCAGGAGGCATGTACCACTGCCACAGTGGCTCCAAGCCCA
CAGAAAAGGGGGCGAATGAGTACGCCTATGCCAAGTGGAAACTCTGTTCTGCTTCAGCAATATGCTT
CATTTTCATGATTGCAGAGGTCGTGGGTGGGCACATTGCTGGGAGTCTTGCTGTTGTCACAGATGCT
GCCCACCTCTTAATTGACCTGACCAGTTTCCTGCTCAGTCTCTTCTCCCTGTGGTTGTCATCGAAGC
CTCCCTCTAAGCGGCTGACATTTGGATGGCACCGAGCACAGGTTTTATTTAGCATTTTATCTCTCAT
CACCCTGGTTGTGGTGACTGGCGTGCTAGTGTACCTGGCATGTGAGCGCCTGCTGTATCCTGATTAC
CAGATCCAGGCGACTGTGATGATCATCGTTTCCAGCTGCGCAGTGGCGGCCGCTAAGAACATTGTTC
TCTCTTTCAGACTAACTGTGGTTTTGCACCAGAGATGCCTTGGCCGCAATCACAAGGAAGTACAAGC
CAATGCCAGCGTCAGAGCTGCTTTTGTGCATGCCCTTGGAGATCTATTTCAGAGTATCAGTGTGCTA
ATTAGTGCACTTATTATCTACTTTAAGCCAGAGTATAAAATAGCCGACCCAATCTGCACATTCATCT
TTTCCATCCTGGTCTTGGCCAGCACCATCTCTATCTTAAAGGACTTCTTCTTCTTACTCATGGAAGG
TGTGCCAAAGAGCCTGAATTACAGTGGTGTGAAAGAGCTTATTTTATCAGTCGACGGGGTGCTGTCT
GTGCACAGCCTGCACATCTGGTCTCTAACAATGAATCAAGTAATTCTCTCAGCTCATGTTGCTACAG
CAGCCAGCCGGGACAGCCAAGTGGTTCGGAGAGAAATTGCTAAAGCCCTTAGCAAAAGCTTTACGAT
GCACTCACTCACCATTCAGATGGAATCTCCAGTTGACCAGGACCCCGACTGCCTTTTCTGTGAAGAC
CCCTGTGAACTAGCTCAGTCACACCGTCAGTTTCCCAAATTTGACAGGCCACCTTCAAACATGCTGC
TATGCAGTTTCTGCATCATAGAAAATAAGGAACCAAAGGAAGAAATTCATGTCATGGTGCAATGCAC
ATTTTATCTATTTATTTAGTTCCATTCACCATGAAGGAAGAGGCACTGAGATCCATCAATCAATTGG
ATTATATACTGATCA

The NOV2b polypeptide (SEQ ID NO:10) encoded by SEQ ID NO:9 is 422 amino acid residues in length, has a molecular weight of 47199.6 Daltons, and is presented using the one-letter amino acid code in Table 2D.

TABLE 2D
NOV2b protein sequence (SEQ ID NO:1O)

MEFLERTYLVNDKAAKMYAFTLESVELQQKPVNKDQCPRERPEELESGGMYHCHSGSKPTEKGANEY
AYAKWKLCSASAICFIFMIAEVVGGHIAGSLAVVTDAAHLLIDLTSFLLSLFSLWLSSKPPSKRLTF
GWHRAQVLFSILSLITLVVVTGVLVYLACERLLYPDYQIQATVMIIVSSCAVAAAKNIVLSFRLTVV
LHQRCLGRNHKEVQANASVRAAFVHALGDLFQSISVLISALIIYFKPEYKIADPICTFIFSILVLAS
TISILKDFFFLLMEGVPKSLNYSGVKELILSVDGVLSVHSLHIWSLTMNQVILSAHVATAASRDSQV
VRREIAKALSKSFTMHSLTIQMESPVDQDPDCLFCEDPCELAQSHRQFPKFDRPPSNMLLCSFCIIE
NKEPKEEIHVMVQCTFYLFI

NOV2c

A NOV2c (alternatively referred to herein as CG57799-02), includes the 1318 nucleotide sequence (SEQ ID NO:11) shown in Table 2E. The disclosed ORF begins with a Kozak consensus ATG initiation codon at nucleotides 51-53 and ends with a TAG codon at nucleotides 1158-1160. Untranslated regions upstream from the initiation codon and downstream from the termination codon are underlined in Table 2E, and the start and stop codons are in bold letters.

TABLE 2E
NOV2c Nucleotide Sequence (SEQ ID NO:11)

AGTGAGTTCAACAACAACGACAACAACAGCCGCAGCTCATCCTGGCCGTCATGGAGTTTCTTGAAAG
AACGTATCTTGTGAATGATAAAGCTGCCAAGATGTATGCTTTCACACTAGAAAGTGTGGAACTCCAA
CAGAAACCGGTGAATAAAGATCAGTGTCCCAGAGAGAGACCAGAGGAGCTGGAGTCAGGAGGCATGT
ACCACTGCCACAGTGGCTCCAAGCCCACAGAAAAGGGGGCGAATGAGTACGCCTATGCCAAGTGGGA
ACTCTGTTCTGCTTCAGCAATATGCTTCATTTTCATGATTGCAGAGGTCGTGGGTGGGCACATTGCT
GGGAGTCTTGCTGTTGTCACAGATGCTGCCCACCTCTTAATTGACCTGACCAGTCTCCTGCTCAGTC
TCTTCTCCCTGTGGTTGTCATCGAAGCCTCCCTCTAAGCGGCTGACATTTGGATGGCACCGAGCAGA
GATCCTTGGTGCCCTGCTCTCCATCCTGTGCATCTGGGTGGTGACTGGCGTGCTAGTGTACCTGGCA
TGTGAGCGCCTGCTGTATCCTGATTACCAGATCCAGGCGACTGTGATGATCATCGTTTCCAGCTGCG
CAGTGGCGGCCAACATTGTACTAACTGTGGTTTTGCACCAGAGATGCCTTGGCCACAATCACAAGGA
AGTACAAGCCAATGCCAGCGTCAGAGCTGCTTTTGTGCATGCCCTTGGAGATCTATTTCAGAGTATC
AGTGTGCTAATTAGTGCACTTATTATCTACTTTAAGCCAGAGTATAAAATAGCCGACCCAATCTGCA
CATTCATCTTTTCCATCCTGGTCTTGGCCAGCACCATCACTATCTTAAAGGACTTCTCCATCTTACT
CATGGAAGGTGTGCCAAAGAGCCTGAATTACAGTGGTGTGAAAGAGCTTATTTTAGCAGTCGACGGG
TTGCTACAGCAGCCAGCCGGGACAGCCAAGTGGTTCGGAGAGAAATTGCTAAAGCCCTTAGCAAAAG
CTTTACGATGCACTCACTCACCATTCAGATGGAATCTCCAGTTGACCAGGACCCCGACTGCCTTTTC
TGTGAAGACCCCTGTGACTAGCTCAGTCACACCGTCAGTTTCCCAAATTTGACAGGCCACCTTCAAA
CATGCTGCTATGCAGTTTCTGCATCATAGAAAATAAGGAACCAAAGGAAGAAATTCATGTCATGGTG
CAATGCATATTTTATCTATTTATTTAGTTCCATTCACCATGAAGG

The NOV2c protein (SEQ ID NO:12) encoded by SEQ ID NO:11 is 369 amino acid residues in length, has a molecular weight of 40721 Daltons, and is presented using the one-letter code in Table 2F.

TABLE 2F
NOV2c protein sequence (SEQ ID NO:12)

MEFLERTYLVNDKAAKMYAFTLESVELQQKPVNKDQCPRERPEELESGGMYHCHSGSKPTE
KGANEYAYAKWELCSASAICFIFMIAEVVGGHIAGSLAVVTDAAHLLIDLTSLLLSLFSLW
LSSKPPSKRLTFGWHRAEILGALLSILCIWVVTGVLVYLACERLLYPDYQIQATVMIIVSS
CAVAANIVLTVVLHQRCLGHNHKEVQANASVRAAFVHALGDLFQSISVLISALIIYFKPEY
KIADPICTFIFSILVLASTITILKDFSILLMEGVPKSLNYSGVKELILAVDGVLSVHSLHI
WSLTMNQVILSAHVATAASRDSQVVRREIAKALSKSFTMHSLTIQMESPVDQDPDCLFCED
PCD

NOV2 Clones

The Psort profile for NOV2 predicts that this polypeptide sequence is likely to be localized at the plasma membrane of 0.6000.

A search against the Patp database, a proprietary database that contains sequences published in patents and patent publications, yielded several homologous proteins shown in Table 2G.

TABLE 2G
Patp results for NOV 2

| | | Smallest |
|---|---|---|
| | | Sum |
| | High | Prob |
| Sequences producing High-scoring Segment Pairs: | Score | P(N) |
| >patp:AAB60094 Human transport protein TPPT-14 | 1623 | 9.3e−167 |
| >patp:AAG22263 Arabidopsis thaliana protein | 307 | 9.5e−56 |
| fragment | | |
| >patp:AAG43478 Aribidopsis thaliana protein | 307 | 9.5e−56 |
| fragment | | |

In a BLAST search of public sequence databases, it was found, for example, that the NOV2b sequence of this invention has 587 of 920 bases (63%) identical to a gb:GENBANK-ID:RNU50927|acc:U50927.1 mRNA from Rattus norvegicus (Rattus norvegicus zinc transporter (ZnT-2) mRNA, complete cds). The full amino acid sequence of the protein of the invention was found to have 165 of 333 amino acid residues (49%) identical to, and 230 of 333 amino acid residues (69%) similar to, the 359 amino acid residue ptnr:SWISSNEW-ACC:Q62941 protein from Rattus norvegicus (Rat) (ZINC TRANSPORTER 2 (ZNT-2)).

Similarly, in a BLAST search of public sequence databases, it was found, for example, that the NOV2c sequence of this invention has 1221 of 1239 bases (98%) identical to a gb:GENBANK-ID:AX061210|acc:AX061210.1 mRNA from Homo sapiens (Sequence 57 from Patent WO0078953). The full amino acid sequence of the protein of the invention was found to have 173 of 333 amino acid residues (51%) identical to, and 235 of 333 amino acid residues (70%) similar to, the 359 amino acid residue ptnr:SWISSNEW-ACC:Q62941 protein from Rattus norvegicus (Rat) (ZINC TRANSPORTER 2 (ZNT-2)).

Additional BLAST results are shown in Table 2H.

TABLE 2H
BLAST results for NOV2

| Gene Index/ | | Length | Identity | Positives | |
|---|---|---|---|---|---|
| Identifier | Protein/Organism | (aa) | (%) | (%) | Expect |
| ptnr: | Zinc | 359 | 174/333 | 234/333 | 2.6 e − |
| SWISS- | transporter 2 | | (52%) | (70%) | 89 |
| NEW- | (ZnT-2) | | | | |
| ACC: | [Rattus | | | | |
| Q629421 | norvegicus] | | | | |
| ptnr: | Zinc transporter | 388 | 159/344 | 223/344 | 1.7 e − |
| SWISS- | 3 (ZnT-3) | | (46%) | (64%) | 78 |
| NEW- | [Mus musculus] | | | | |
| ACC: | | | | | |
| P97441 | | | | | |
| ptnr: | 6 Zinc | 388 | 162/376 | 237/376 | 3.2 e − |
| SWISS- | transporter 3 | | (43%) | (63%) | 75 |
| NEW- | (ZnT-3) | | | | |
| ACC: | [Homo sapiens] | | | | |
| Q9972 | | | | | |

A multiple sequence alignment is given in Table 2I, with the NOV2 protein of the invention being shown on line 1, in a ClustalW analysis comparing NOV2 with related protein sequences is disclosed in Table 2H. The homologies shared by NOV2a, NOV2b, and NOV2c polypeptides are also shown in Table 2I.

TABLE 2I
Information for the ClustalW proteins:

1. >NOV2a; SEQ ID NO:8
2. >NOV2b; SEQ ID NO:10
3. >NOV2c; SEQ ID NO:12
4. >Q62941/Zinc transporter 2 (ZnT-2) [Rattus norvegicus]; SEQ ID NO:37
5. >P97441/Zinc Tranporter 3 (ZnT-3) [Mus musculus]; SEQ ID NO:38
6. >Q99726/Zinc Transporter 3 (ZnT-3) [Homo sapiens] SEQ ID NO:39

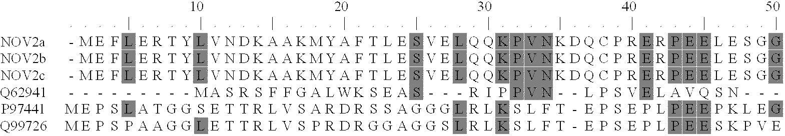

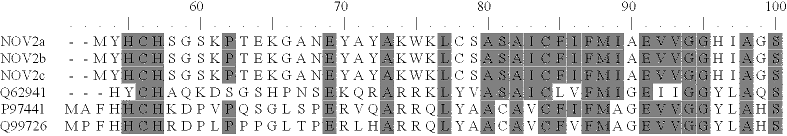

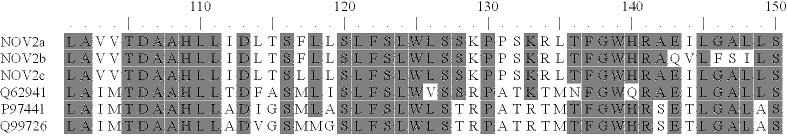

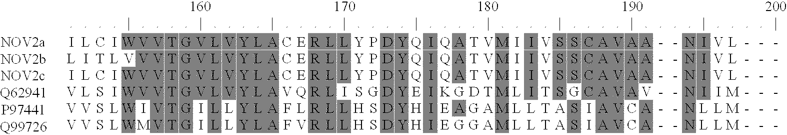

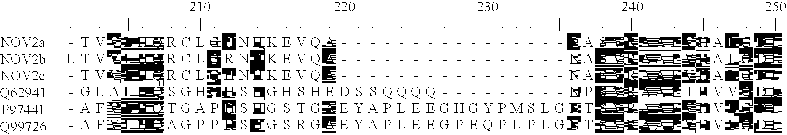

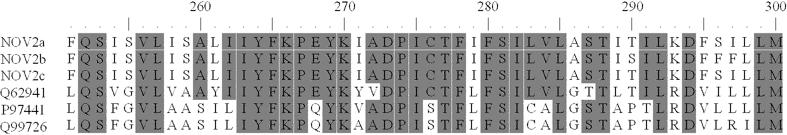

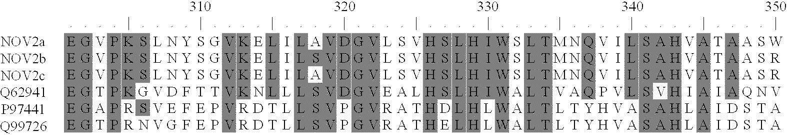

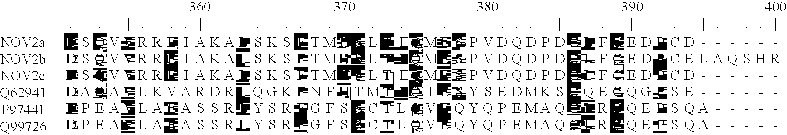

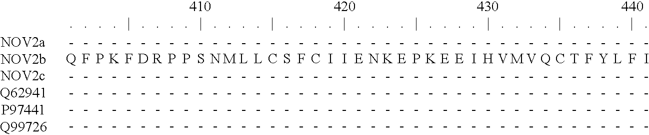

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 2J lists the domain description from DOMAIN analysis results against NOV2.

TABLE 2J
Domain Analysis of NOV 2

| | Region of | | |
|---|---|---|---|
| Model | Homology | Score (bits) | E value |
| Cation Efflux | 127 to 361 | 221.1 | 1.6e−62 |

The presence of protein regions on NOV2 that are homologous to the Cation Efflux domain (IPR002524) is consistent with the organization of members of the ZNT Protein Family. This indicates that the NOV2 sequence has properties similar to those of other Cation Efflux proteins known to contain these domains.

The NOV2 ZNT-like gene is expressed in at least the following tissues: pancreas, bone marrow, cartilage, placenta, and kidney. The expression pattern, map location, domain analysis, and protein similarity information for the invention suggest that this NOV2 may function as a ZNT-like protein.

The NOV2 nucleic acids and proteins of the invention, therefore, are useful in potential therapeutic applications implicated, for example but not limited to, in various pathologies/disorders as described below and/or other pathologies/disorders. For example, the compositions of the present invention will have efficacy for the treatment of patients suffering from: cancer, trauma, regeneration (in vitro and in vivo), viral/bacterial/parasitic infections, fertility as well as other diseases, disorders and conditions. Potential therapeutic uses for the invention(s) are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

By way of non-limiting example, the compositions of the present invention will have efficacy for treatment of patients suffering from diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, pancreatitis, obesity, hemophilia, hypercoagulation, idiopathic thrombocytopenic purpura, allergies, immunodeficiencies, transplantation, graft versus host, arthritis,tendinitis, T cell proliferative disorders and diseases, zinc toxicity as well as other diseases, disorders and conditions. A cDNA encoding the ZNT-like protein may be useful in gene therapy, and the ZNT-like protein may be useful when administered to a subject in need thereof. The novel nucleic acid encoding the ZNT-like protein, and the ZNT-like protein of the invention, or fragments thereof, may further be useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. The disclosed NOV2 protein has multiple hydrophilic regions, each of which can be used as an immunogen. In one embodiment, a contemplated NOV2 epitope is from about amino acids 10 to 75. In another embodiment, a NOV2 epitope is from about amino acids 100 to 150. In additional embodiments, NOV2 epitopes are from about amino acids 175 to 250, and from about amino acids 310 to 410.

NOV3

A NOV3 polypeptide is a Mitsugumin29-like protein (MG29). The NOV1 nucleic acid sequences disclosed herein map to chromosome 3. The nucleic acid sequence (and encoded polypeptide) of two NOV3 sequences - NOV3a, and NOV3b are provided.

NOV3a

A NOV3a (alternatively referred to herein as SC126413398_A), includes the 854 nucleotide sequence (SEQ ID NO:13) and which encodes a novel MG29-like protein is shown in Table 3A. The disclosed ORF begins with a Kozak consensus ATG initiation codon at nucleotides 2-4 and ends with a TAA codon at nucleotides 803-805. Untranslated regions upstream from the initiation codon and downstream from the termination codon are underlined in Table 3A, and the start and stop codons are in bold letters.

TABLE 3A
NOV3a Nucleotide Sequence (SEQ ID NO:13)

CATGTCCTCGACCGAGAGCGCCGGCCGCACGGCGGACAAGTCGCCGCGCCAGCAGGTAGACCGCCTA
CTCGTGGGGCTGCGCTGGCGGCGGCTGGAGGAGCCGCTGGGCTTCATCAAAGTTCTCCAGTGGCTCT
TTGCTATTTTCGCCTTCGGGTCCTGTGGCTCCTACAGCGGGGAGACAGGAGCAATGGTTCGCTGCAA
CAACGAAGCCAAGGACGTGAGCTCCATCATCGTTGCATTTGGCTATCCCTTCAGGTTGCACCGGATC
CAATATGAGATGCCCCTCTGCGATGAAGAGTCCAGCTCCAAGACCATGCACCTCATGGGGGACTTCT
CTGCACCCGCCGAGTTCTTCGTGACCCTTGGCATCTTTTCCTTCTTCTATACCATGGCTGCCCTAGT
TATCTACCTGCGCTTCCACAACCTCTACACAGAGAACAAACGCTTCCCGCTGGTGGACTTCTGTGTG
ACTGTCTCCTTCACCTTCTTCTGGCTGGTAGCTGCAGCTGCCTGGGGCAAGGGCCTGACCGATGTCA
AGGGGGCCACACGACCATCCAGCTTGACAGCAGCCATGTCAGTGTGCCATGGAGAGGAAGCAGTGTG
CAGTGCCGGGGCCACGCCCTCTATGGGCCTGGCCAACATCTCCGTGGTGCTCTTTGGCTTTATCAAC
TTCTTCCTGTGGGCCGGGAACTGTTGGTTTGTGTTCAAGGAGACCCCGTGGCATGGACAGGGCCAGG
ACCAGGACCAGGGCCAGGGTCCCAGCCAGGAGAGTGCAGCTGAGCAGGGAGCAGTGGAGAAGCAGTA
AGCAGCCCCCCACCTGGCTATTCCCGAACTGGACAGCACCTCTTCAACCA

Variant sequences of NOV3a are included in Example 2, Table 51. A variant sequence can include a single nucleotide polymorphism (SNP). A SNP can, in some instances, be referred to as a “cSNP” to denote that the nucleotide sequence containing the SNP originates as a cDNA.

The NOV3a polypeptide (SEQ ID NO:14) encoded by SEQ ID NO:13 is 267 amino acid residues in length, has a molecular weight of 29583.5 Daltons, and is presented using the one-letter amino acid code in Table 3B.

TABLE 3B
NOV3a protein sequence (SEQ ID NO:14)

MSSTESAGRTADKSPRQQVDRLLVGLRWRRLEEPLGFIKVLQWLFAIFAFGSCGSYSGETGAMVRCN
NEAKDVSSIIVAFGYPFRLHRIQYEMPLCDEESSSKTMHLMGDFSAPAEFFVTLGIFSFFYTMAALV
IYLRFHNLYTENKRFPLVDFCVTVSFTFFWLVAAAAWGKGLTDVKGATRPSSLTAAMSVCHGEEAVC
SAGATPSMGLANISVVLFGFINFFLWAGNCWFVFKETPWHGQGQDQDQGQGPSQESAAEQGAVEKQ

NOV3b

A NOV3b (alternatively referred to herein as CG55861-02), includes the 642 nucleotide sequence (SEQ ID NO:15) shown in Table 3C. The disclosed ORF begins with a Kozak consensus ATG initiation codon at nucleotides 2-4 and ends with a TAA codon at nucleotides 626-628. Untranslated regions upstream from the initiation codon and downstream from the termination codon are underlined in Table 3C, and the start and stop codons are in bold letters.

TABLE 3C
NOV3b Nucleotide Sequence (SEQ ID NO:15)

CATGTCCTCGACCGAGAGCGCCGGCCGCACGGCGGACAAGTCGCCGCGCCAGCAGGTGGACCGCCTA
CTCGTGGGGCTGCGCTGGCGGCGGCTGGAGGAGCCGCTGGGCTTCATCAAAGTTCTCCAGTGGCTCT
TTGCTATTTTCGCCTTCGGGTCCTGTGGCTCCTACAGCGGGGAGACAGGAGCAATGGTTCGCTGCAA
CAACGAAGCCAAGGACGTGAGCTCCATCATCGTTGCATTTGGCTATCCCTTCAGGTTGCACCGGATC
CAATATGAGATGCCCCTCTGCGATGAAGAGTCCAGCTCCAAGACCATGCACCTCATGGGGGACTTCT
CTGCACCCGCCGAGTTCTTCGTGACCCTTGGCATCTTTTCCTTCTTCTATACCATGGCTGCCCTAGT
TATCTACCTGCGCTTCCACAACCTCTACACAGAGAACAAACGCTTCCCGCTGGTGCTCTTTGGCTTT
ATCAACTTCTTCCTGTGGGCCGGGAACTGTTGGTTTGTGTTCAAGGAGACCCCGTGGCATGGACAGG
GCCAGGGCCAGGACCAGGACCAGGACCAGGGCCAGGGCCAGGGTCCCAGCCAGGAGAGTGCAGCTGA
GCAGGGAGCAGTGGAGAAGCAGTAAGCAGCCCCCCACCT

The NOV1b protein (SEQ ID NO:16) encoded by SEQ ID NO:15 is 208 amino acid residues in length, has a molecular weight of 23618.6 Daltons, and is presented using the one-letter code in Table 3D.

TABLE 3D
NOV3b protein sequence (SEQ ID NO:16)

MSSTESAGRTADKSPRQQVDRLLVGLRWRRLEEPLGFIKVLQWLFAIFAFGSCGSYSGETGAMVRCN
NEAKDVSSIIVAFGYPFRLHRIQYEMPLCDEESSSKTMHLMGDFSAPAEFFVTLGIFSFFYTMAALV
IYLRFHNLYTENKRFPLVLFGFINFFLWAGNCWFVFKETPWHGQGQGQDQDQDQGQGQGPSQESAAE
QGAVEKQ

NOV3 Clones

The Psort profile for NOV3a predicts that this polypeptide sequence is likely to be localized in the plasma membrane with a certainty of 0.6000.The Psort profile for NOV3b predicts that this polypeptide sequence is likely to be localized in the plasma membrane with a certainty of 0.4400. The Signal P predicts a likely cleavage site for a NOV3 polypeptide is between positions 57 and 58, i.e., at the dash in the sequence SYS-GE.

A search against the Patp database, a proprietary database that contains sequences published in patents and patent publications, yielded several homologous proteins shown in Table 3E.

TABLE 3E
Patp results for NOV 3

| | | Smallest |
|---|---|---|
| | | Sum |
| | High | Prob |
| Sequences producing High-scoring Segment Pairs: | Score | P(N) |
| >patp:AAY29817 Human synapse related glyco- | 564 | 1.5e−54 |
| protein 2 | | |
| >patp:AAG03792 Human secreted protein, SEQ | 272 | 1.3e−23 |
| ID:7873 | | |

In a BLAST search of public sequence databases, it was found, for example, that the nucleic acid sequence of NOV1a has 725 of 801 bases (90%) identical to a MG29 mRNA from Oryctolagus cuniculus (GENBANK-ID: AB004816). The full amino acid sequence of the protein of the invention was found to have 254 of 267 amino acid residues (95%) identical to, and 258 of 267 amino acid residues (96%) similar to, the 264 amino acid residue MG29 protein from Oryctolagus cuniculus (Rabbit) (O62646).

Similarly, in a BLAST search of public sequence databases, it was found, for example, that the nucleic acid sequence of NOV3b has 511 of 617 bases (82%) identical to a gb:GENBANK-ID:AB004816|acc:AB004816.1 mRNA from Oryctolagus cuniculus (Oryctolagus cuniculus mRNA for MG29, complete cds). The full amino acid sequence of the protein of the invention was found to have 148 of 171 amino acid residues (86%) identical to, and 153 of 171 amino acid residues (89%) similar to, the 264 amino acid residue ptnr:SPTREMBL-ACC:O62646 protein from Oryctolagus culliculus (Rabbit) (MG29).

Additional BLAST results are shown in Table 3F.

TABLE 3F
BLAST results for NOV3

| Gene Index/ | Protein/ | Length | Identity | Positives | |
|---|---|---|---|---|---|
| Identifier | Organism | (aa) | (%) | (%) | Expect |
| ptnr: | MG29 | 264 | 254/267 | 258/267 | 1.8 e − |
| SPTREMBL- | [Oryctolagus | | (95%) | (96%) | 136 |
| ACC:062646 | cuniculus] | | | | |
| ptnr: | MG29 [Mus | 264 | 248/267 | 260/267 | 3.9 e − |
| SPTREMBL- | musculus] | | (92%) | (97%) | 134 |
| ACC:089104 | | | | | |
| ptnr: | SYNAP- | 307 | 110/222 | 145/222 | 1.8 e − |
| SWISSPROT- | TOPHYSIN | | (49%) | (65%) | 56 |
| ACC:P20488 | (MAJOR | | | | |
| | SYNAPTIC | | | | |
| | VESICLE | | | | |
| | PROTEIN P38) | | | | |
| | [Bos taurus] | | | | |

A multiple sequence alignment is given in Table 3G, with the NOV3 protein of the invention being shown on line 1, in a ClustalW analysis comparing NOV3 with related protein sequences is disclosed in Table 3F. The homologies shared by NOV3a and NOV3b polypeptides are also shown in Table 3G.

TABLE 3G
Information for the ClustalW proteins:

1. >NOV3a; SEQ ID NO:14
2. >NOV3b; SEQ ID NO:16
3. >O62646/MG29 [Oryctolagus cuniculus]; SEQ ID NO:40
4. >O89104/MG29 [Mus musculus]; SEQ ID NO:41

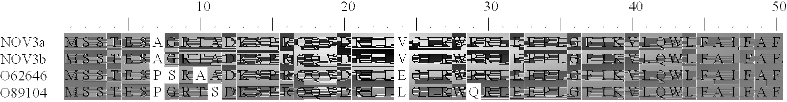

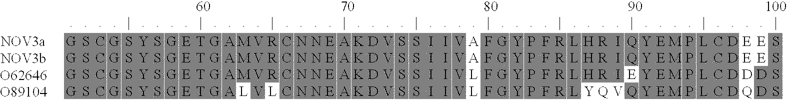

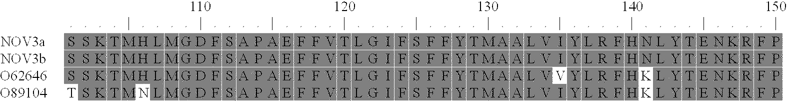

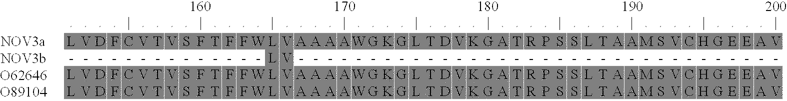

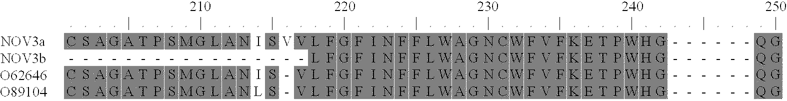

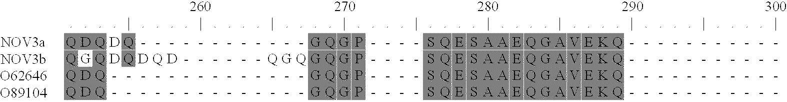

The presence of identifiable domains in the protein disclosed herein was determined by searches using algorithms such as PROSITE, Blocks, Pfam, ProDomain, Prints and then determining the Interpro number by crossing the domain match (or numbers) using the Interpro website (http:www.ebi.ac.uk/interpro/). Table 3H lists the domain description from DOMAIN analysis results against NOV3.

TABLE 3H
Domain Analysis of NOV 3

| | Region of | | |
|---|---|---|---|
| Model | Homology | Score (bits) | E value |
| Synaptophysin | 27 to 208 | 80 | 4.7e−20 |

The presence of protein regions on NOV3 that are homologous to the synaptophysin domain (IPR11111) is consistent with the organization of members of the MG29 Protein Family. This indicates that the NOV3 sequence has properties similar to those of other synaptophysin domain-containing proteins.

The NOV3 MG29-like gene is expressed in at least in the heart and the brain. The expression pattern, map location, domain analysis, and protein similarity information for the invention suggest that this NOV3 may function as a MG29-like protein.

The NOV3 nucleic acids and proteins of the invention, therefore, are useful in potential therapeutic applications implicated, for example but not limited to, in various pathologies/disorders as described below and/or other pathologies/disorders. For example, the compositions of the present invention will have efficacy for the treatment of patients suffering from: cancer, trauma, regeneration (in vitro and in vivo), viral/bacterial/parasitic infections, fertility as well as other diseases, disorders and conditions. Potential therapeutic uses for the invention(s) are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

By way of non-limiting example, the compositions of the present invention will have efficacy for treatment of patients suffering from Wiskott-Aldrich syndrome, Aldrich Syndrome, Eczema-Thrombocytopenia-Immunodeficiency Syndrome, Thrombocytopenia, Night Blindness, Amyotrophic lateral sclerosis, Batten disease, Ceroid Lipofuscinosis, Rett syndrome, Pick disease (lobar atrophy). A cDNA encoding the NOV3 protein may be useful in gene therapy, and the MG29-like protein may be useful when administered to a subject in need thereof. The novel nucleic acid encoding the MG29-like protein, and the MG29-like protein of the invention, or fragments thereof, may further be useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. The disclosed NOV3 protein has multiple hydrophilic regions, each of which can be used as an immunogen. In one embodiment, a contemplated NOV3 epitope is from about amino acids 1 to 4. In another embodiment, a NOV3 epitope is from about amino acids 50 to 75. In additional embodiments, NOV3 epitopes are from about amino acids 125 to 170, from about amino acids 171 to 200, and from about amino acids 225 to 267.

NOV4

NOV4 includes two novel Slit3-like proteins disclosed below. The nucleic acid sequence (and encoded polypeptide) of two NOV4 sequences - NOV4a and NOV4b are provided.

NOV4a

A disclosed NOV4a (also referred to as 20760813.0.10) nucleic acid of 2380 nucleotides (SEQ ID NO:17) encoding a novel Slit3-like protein is shown in Table 4A. An open reading frame was identified beginning with an ATG initiation codon at nucleotides 237-239 and ending with a TGA codon at nucleotides 2055-2057. Untranslated regions upstream from the initiation codon and downstream from the termination codon are underlined in Table 4A. The start and stop codons are in bold letters.

TABLE 4A
NOV4a Nucleotide Sequence (SEQ ID NO:17)

GCTACGTCTTGTAAAACTATGATTAGCATTGCACTCCTCTCACTGCCGTTGAATGGACCTTGGCAGC
AGAGACAGTAGAGAAAGGCAGTAGAGAAGGTTAGAACCTAGAAGACTCTAACTTTGATTAACTTTTT
TTTTTTTATCCTTGAGGATAAATCATGAGGAACCTATAACCCTTTTGGCCACATGCAAAAAAGCAAG
ACCCGTGACCAAGGTGTAGACTAAGAAGTGGAGTCATGCTTCACACGGCCATATCATGCTGGCAGCC
ATTCCTGGGTCTGGCTGTGGTGTTAATCTTCATGGGATCCACCATTGGCTGCCCCGCTCGCTGTGAG
TGCTCTGCCCAGAACAAATCTGTTAGCTGTCACAGAAGGCGATTGATCGCCATCCCAGAGGGCATTC
CCATCGAAACCAAAATCTTGGACCTCAGTAAAAACAGGCTAAAAAGCGTCAACCCTGAAGAATTCAT
ATCATATCCTCTGCTGGAAGAGATAGACTTGAGTGACAACATCATTGCCAATGTGGAACCAGGAGCA
TTCAACAATCTCTTTAACCTGCGTTCCCTCCGCCTAAAAGGCAATCGTCTAAAGCTGGTCCCTTTGG
GAGTATTCACGGGGCTGTCCAATCTCACTAAGCTTGACATTAGTGAGAATAAGATTGTCATTTTACT
AGACTACATGTTCCAAGATCTACATAACCTGAAGTCTCTAGAAGTGGGGGACAATGATTTGGTTTAT
ATATCACACAGGGCATTCAGTGGGCTTCTTAGCTTGGAGCAGCTCACCCTGGAGAAATGCAACTTAA
CAGCAGTACCAACAGAAGCCCTCTCCCACCTCCGCAGCCTCATCAGCCTGCATCTGAAGCATCTCAA
TATCAACAATATGCCTGTGTATGCCTTTAAAAGATTGTTCCACCTGAAACACCTAGAGATTGACTAT
TGGCCTTTACTGGATATGATGCCTGCCAATAGCCTCTACGGTCTCAACCTCACATCCCTTTCAGTCA
CCAACACCAATCTGTCTACTGTACCCTTCCTTGCCTTTAAACACCTGGTATACCTGACTCACCTTAA
CCTCTCCTACAATCCCATCAGCACTATTGAAGCAGGCATGTTCTCTGACCTGATCCGCCTTCAGGAG
CTTCATATAGTGGGGGCCCAGCTTCGCACCATTGAGCCTCACTCCTTCCAAGGGCTCCGCTTCCTAC
GCGTGCTCAATGTGTCTCAGAACCTGCTGGAAACTTTGGAAGAGAATGTCTTCTCCTCCCCTAGGGC
TCTGGAGGTCTTGAGCATTAACAACAACCCTCTGGCCTGTGACTGCCGCCTTCTCTGGATCTTGCAG
CGACAGCCCACCCTGCAGTTTGGTGGCCAGCAACCTATGTGTGCTGGCCCAGACACCATCCGTGAGA
GGTCTTTCAAGGATTTCCATAGCACTGCCCTTTCTTTTTACTTTACCTGCAAAAAACCCAAAATCCG
TGAAAAGAAGTTGCAGCATCTGCTAGTAGATGAAGGGCAGACAGTCCAGCTAGAATGCAGTGCAGAT
GGAGACCCGCAGCCTGTGATTTCCTGGGTGACACCCCGAAGGCGTTTCATCACCACCAAGTCCAATG
GAAGAGCCACCGTGTTGGGTGATGGCACCTTGGAAATCCGCTTTGCCCAGGATCAAGACAGCGGGAT
GTATGTTTGCATCGCTAGCAATGCTGCTGGGAATGATACCTTCACAGCCTCCTTAACTGTGAAAGGA
TTCGCTTCAGATCGTTTTCTTTATGCGAACAGGACCCCTATGTACATGACCGACTCCAATGACACCA
TTTCCAATGGCAGCAATGCCAATACTTTTTCCCTGGACCTTAAAACAATACTGGTGTCTACAGCTAT
GGGCTGCTTCACATTCCTGGGAGTGGTTTTATTTTGTTTTCTTCTCCTTTTTGTGTGGAGCCGAGGG
AAAGGCAAGCACAAAAACAGCATTGACCTTGAGTATGTGCCCAAAAAAAACCATGGTGCTGTTGTGG
AAGGGGAGGTAGCTGGACCCAGGAGGTTCAACATGAAAATGATTTGAAGGCCCACCCCTCACATTAC
TGTCTCTTTGTCAATGTGGGTAATCAGTAAGACAGTATGGCACAGTAAATTACTAGATTAAGAGGCA
GCCATGTGCAGCTGCCCCTGTATCAAAAGCAGGGTCTATGGAAGCAGGAGGACTTCCAATGGAGACT
CTCCATCGAAAGGCAGGCAGGCAGGCATGTGTCAGAGCCCTTCACACAGTGGGATACTAAGTGTTTG
CGTTGCAAATATTGGCGTTCTGGGGATCTCAGTAATGAACCTGAATATTTGGCTCACACTCACGGAC
AATTATTCAGCATTTTCTACCACTGCAAAAAAAAA

Variant sequences of NOV4a are included in Example 2, Table 52. A variant sequence can include a single nucleotide polymorphism (SNP). A SNP can, in some instances, be referred to as a “cSNP” to denote that the nucleotide sequence containing the SNP originates as a cDNA.

The NOV4a protein (SEQ ID NO:18) encoded by SEQ ID NO:17 is 606 amino acid residues in length, has a molecular weight of 68046 Daltons, and is presented using the one-letter amino acid code in Table 4B.

TABLE 4B
Encoded NOV4a protein sequence

(SEQ ID NO:18)

MLHTAISCWQPFLGLAVVLIFMGSTIGCPARCECSAQNKSVSCHRRRLIAIPEGIPIETKILDLS
KNRLKSVNPEEFISYPLLEEIDLSDNIIANVEPGAFNNLFNLRSLRLKGNRLKLVPLGVFTGLSN
LTKLDISENKIVILLDYMFQDLHNLKSLEVGDNDLVYISHRAFSGLLSLEQLTLEKCNLTAVPTE
ALSHLRSLISLHLKHLNINNMPVYAFKRLFHLKHLEIDYWPLLDMMPANSLYGLNLTSLSVTNTN
LSTVPFLAFKHLVYLTHLNLSYNPISTIEAGMFSDLIRLQELHIVGAQLRTIEPHSFQGLRFLRV
LNVSQNLLETLEENVFSSPRALEVLSINNNPLACDCRLLWILQRQPTLQFGGQQPMCAGPDTIRE
RSFKDFHSTALSFYFTCKKPKIREKKLQHLLVDEGQTVQLECSADGDPQPVISWVTPRRRFITTK
SNGRATVLGDGTLEIRFAQDQDSGMYVCIASNAAGNDTFTASLTVKGFASDRFLYANRTPMYMTD
SNDTISNGSNANTFSLDLKTILVSTAMGCFTFLGVVLFCFLLLFVWSRGKGKHKNSIDLEYVPKK
NHGAVVEGEVAGPRRFNMKMI

NOV4b

A disclosed NOV4b nucleic acid (also referred to as CG51514-05) of 2187 nucleotides (SEQ ID NO:19) encoding a novel Slit3-like protein is shown in Table 4D. An open reading frame was identified beginning with an ATG initiation codon at nucleotides 83-85 and ending with a TGA codon at nucleotides 1901-1903. Untranslated regions upstream from the initiation codon and downstream from the termination codon are underlined in Table 4C. The start and stop codons are in bold letters.

TABLE 4C
NOV4b Nucleotide Sequence

(SEQ ID NO:19)

AATCATGAGGAACCTATAACCCTTTTGGCCACATGCAAAAAAGCAAGACCCGTGACCAAGGTGTAGACTAAGAA
GTGGAGTCATGCTTCACACGGCCATATCATGCTGGCAGCCATTCCTGGGTCTGGCTGTGGTGTTAATCTTCATG
GGACCCACCATTGGCTGCCCCGCTCGCTGTGAGTGCTCTGCCCAGAACAAATCTGTTAGCTGTCACAGAAGGCG
ATTGATCGCCATCCCAGAGGGCATTCCCATCGAAACCAAAATCTTGAACCTCAGTAAAAACAGGCTAAAAAGCG
TCAACCCTGAAGAATTCATATCATATCCTCTGCTGGAAGAGATAGACTTGAGTGACAACATCATTGCCAATGTG
GAACCAGGAGCATTCAACAATCTCTTTAACCTGCGTTCCCTCCGCCTAAAAGGCAATCGTCTAAAGCTGGTCCC
TTTGGGAGTATTCACGGGGCTGTCCAATCTCACTAACCTTGACATTAGTGAGAATAAGATTGTCATTTTACTAG
ACTACATGTTCCAAGATCTACATAACCTGAAGTCTCTAGAAGTGGGGGACAATGATTTGGTTTATATATCACAC
AGGGCATTCAGTGGGCTTCTTAGCTTGGAGCAGCTCACCCTGGAGAAATGCAACTTAACAGCAGTACCAACAGA
AGCCCTCTCQCACCTCCGCAGCCTCATCAGCCTGCATCTGAAGCATCTCAATATCAACAATATGCCTGTGTATA
CCTTTAAAAGATTGTTCCACCTGAAACACCTAGAGATTGACTATTGGCCTTTACTGGATATGATGCCTGCCAAT
AGCCTCTACGGTCTCAACCTCACACCCCTTTCAGTCACCAACACCAATCTGTCTACTGTACCCTTCCTTGCCTT
TAAACACCTGGTATACCTGACTCACCTTAACCTCTCCTACAATCCCATCAGCACTATTGAAGCAGGCATGTTCT
CTGACCTGATCCGCCTTCAGGAGCTTCATATAGTGGGGGCCCAGCTTCGCACCATTGAGCCTCACTCCTTCCAA
CGGCTCCGCTTCCTACGCGTGCTCAATGTGTCTCAGAACCTGCTGGAAACTTTGGAAGAGAATGTCTTCTCCTC
CCCTAGGGCTCTGGAGGTCTTGAGCATTAACAACAACCCTCTGGCCTGTGACTGCCGCCTTCTCTGGATCTTGC
AGCGACAGCCCACCCTGCAGTTTGGTOGCCAGCAACCTATGTGTGCTGGCCCAGACACCATCCGTGAGAGGTCT
TTCAAGGATTTCCATAGCACTGCCCTTTCTTTTTACTTTACCTGCAAAAAACCCAAAATCCGTGAAAAGAAGTT
GCAGCATCTGCTAGTAGATGAAGGGCAGACAGTCCAGCTAGAATGCAGTGCAGATGGACACCCGCAGCCTGTGA
TTTCCTGGGTGACACCCCGAAGGCGTTTCATCACCACCAAGTCCAATGGAAGAGCCACCGTGTTGGGTGATGGC
ACCTTGGAAATCCGCTTTGCCCAGGATCAAGACAGCCGGATGTATGTTTCCATCCCTA&CAATGCTQCTGGGAA
TGATACCTTCACAGCCTCCTTAACTGTGAAAGGATTCGCTTCACATCGTTTTCTTTATGCGAACAGGACCCCTA
TCTACATGACCGACTCCAATGACACCATTTCCAATGCCACCAATCCCAATACTTTTTCCCTGGACCTTAAAACA
ATACTGGTGTCTACAGCTATGGGCTGCTTCACATTCCTGGGAGTGGTTTTATTTTGTTTTCTTCTCCTTTTTGT
GTGGAGCCGAGGGAAAGGCAAGCACAAAAACAGCATTGACCTTGAGTATGTGCCCAGAAAAAACAGTGGTGCTG
TTGTCGAAGGGGAGGTAGCTGGACCCAGGAGGTTCAACATGAAAATGATTTGAAGGCCCACCCCTCACATTACT
GTCTCTTTCTCAATGTGCGTAATCAGTAAGACAGTATGGCACAGTAAATTACTAGATTAAGAGGCAGCCATGTG
CAGCTGCCCCTGTATCAAAAGCAGGGTCTATGGAAGCAGGAGGACTTCCAATGGAGACTCTCCATCGAAAGGCA
GGAGGCAGGCATGTGTCAGAGCCCTTCACACAGTGGGATACTAAGTGTTTGCCTTCCAAATATTGGCGTTCTG
GGCATCTCAGTAATGAACCTGAATATTTGGCTCACACTCAC

Variant sequences of NOV4b are included in Example 2, Table 53. A variant sequence can include a single nucleotide polymorphism (SNP). A SNP can, in some instances, be referred to as a “cSNP” to denote that the nucleotide sequence containing the SNP originates as a cDNA.

The NOV4b protein (SEQ ID NO:20) encoded by SEQ ID NO:19 is 606 amino acid residues in length, and is presented using the one-letter amino acid code in Table 4D.

TABLE 4D
Encoded NOV4b protein sequence

(SEQ ID NO:20)

MLHTAISCWQPFLGLAVVLIFMGPTIGCPARCECSAQNKSVSCHRRRLIAIPEGIPIETKILNLS
KNRLKSVNPEEFISYPLLEEIDLSDNIIANVEPGAFNNLFNLRSLRLKGNRLKLVPLGVFTGLSN
LTKLDISENKIVILLDYMFQDLHNLKSLEVGDNDLVYISHRAFSGLLSLEQLTLEKCNLTAVPTE
ALSHLRSLISLHLKHLNINNMPVYTFKRLFHLKHLEIDYWPLLDMMPANSLYGLNLTPLSVTNTN
LSTVPFLAFKHLVYLTHLNLSYNPISTIEAGMFSDLIRLQELHIVGAQLRTIEPHSFQGLRFLRV
LNVSQNLLETLEENVFSSPRALEVLSINNNPLACDCRLLWILQRQPTLQFGGQQPMCAGPDTIRE
RSFKDFHSTALSFYFTCKKPKIREKKLQHLIVDEGQTVQLECSADGDPQPVISWVTPRRRFITTK
SNGRATVLGDGTLEIRFAQDQDSGMYVCIASNAAGNDTFTASLTVKGFASDRFLYANRTPMYMTD
SNDTISNGTNANTFSLDLKTILVSTAMGCFTFLGVVLFCFLLLFVWSRGKGKHKNSIDLEYVPRK
NSGAVVEGEVAGPRRFNMKMI

NOV4 Clones

The Psort profile for NOV4 predicts that these sequences have a signal peptide and are likely to be localized at the plasma membrane with a certainty of 0.4600. In other embodiments, NOV4 localizes to the endoplasmic reticulum (membrane) with a certainty of 0.1000, to the endoplasmic reticulum (lumen) with a certainty of 0.1000, or extracellularly with a certainty of 0.1000. The Signal P predicts a likely cleavage site for a NOV4 peptide is between positions 27 and 28, i.e., at the dash in the sequence TIG-CP.

A search against the Patp database, a proprietary database that contains sequences published in patents and patent publications, yielded several homologous proteins shown in Table 4E.

TABLE 4E
Patp Results for NOV 4

| | | Smallest |
|---|---|---|
| | | Sum |
| | High | Prob |
| Sequences producing High-scoring Segment Pairs: | Score | P(N) |
| patp:AAB31161 Amino acid sequence of a human | 2137 | 3.2e−221 |
| TOLL protein | | |
| patp:AAB74705 Human membrane associated pro- | 1941 | 1.9e−200 |
| tein MEMAP-11 | | |
| patp:AAW84596 Amino acid sequence of the | 1931 | 2.1e−199 |
| human Tango-79 | | |
| patp:AAY13357 Amino acid sequence of pro- | 1927 | 5.7e−199 |
| tein PR0227 | | |

In a BLAST search of public sequence databases, it was found, for example, that the full amino acid sequence of the protein of the invention was found to have 603 of 606 (99%) amino acid residues identical to, and 606 of 606 (100%) residues positive with, the 606 amino acid residue protein from Homo Sapiens (ptnr:SPTREMBL-ACC:Q9BZ20).

NOV4 has homology to the proteins shown in the BLASTP data in Table 4F.

TABLE 4F
BLAST results for NOV4

| Gene Index/ | | Length | Identity | Positives | |
|---|---|---|---|---|---|
| Identifier | Protein/ Organism | (aa) | (%) | (%) | Expect |
| Q9BZ20 | ba438b23.1 | 606 | 544/606 | 547/606 | 0.0 |
| | Neuronal leucine- | | (90%) | (90%) | |
| | rich repeat | | | | |
| | protein | | | | |
| | [Homo sapiens] | | | | |
| Q9ESY6 | Neuronal leucine- | 707 | 131/538 | 216/538 | 3 e − 37 |
| | rich repeat | | (24%) | (40%) | |
| | protein-3 | | | | |
| | [Rattus | | | | |
| | norvegicus] | | | | |
| Q9HBW1 | Nag14 | 649 | 125/477 | 189/477 | 1 e − 31 |
| | [Homo sapiens] | | (26%) | (40%) | |
| Q9WVB4 | Slit3 (fragment) | 1523 | 69/222 | 103/222, | 2 e − 19 |
| | [Mus musculus] | | (31%) | (46%) | |

A multiple sequence alignment is given in Table 4G, with the NOV4a and NOV4b being shown on line 1 and line 2, respectively. This Clustal W analysis compares the NOV4 protein with the related protein sequences shown in Table 4F. The homologies shared by NOV4a and NOV4b polypeptides are also shown in Table 4G.

TABLE 4G
ClustalW Analysis of NOV4

1. >NOV4a; SEQ ID NO:18
2. >NOV4b; SEQ ID NO:20
3. >Q9BZ20/BA438B23.1 Neutronal leucine-rich repeat protein [Homo sapiens]; SEQ ID NO:42
4. >Q9ESY6/Neuronal leucine-rich repeat protein-3 [Rattus norvegicus]; SEQ ID NO:43
5. >Q9HBW1/Nag14 [Homo sapiens]; SEQ ID NO:44
6. >Q9WVB4/Slit3 (fragment) [Mus musculus]; SEQ ID NO:45

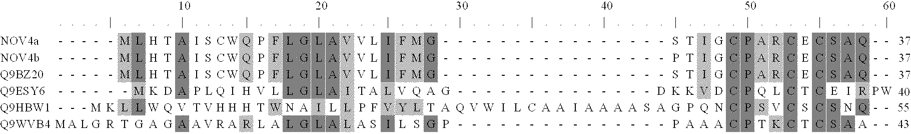

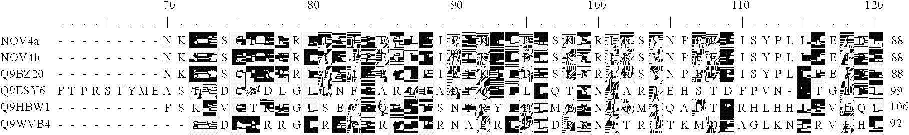

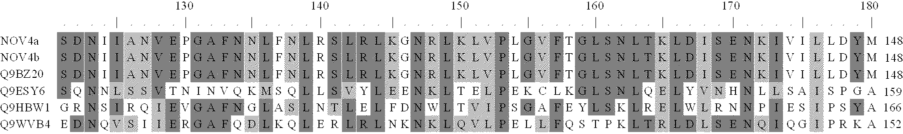

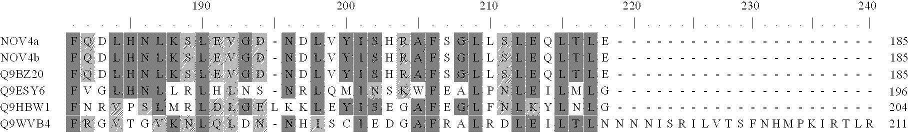

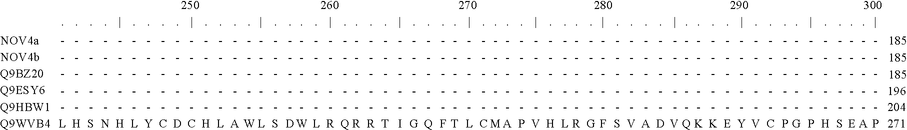

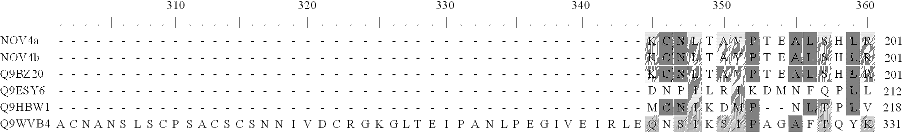

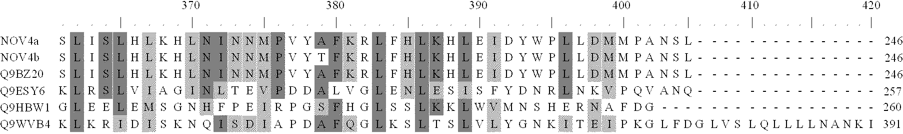

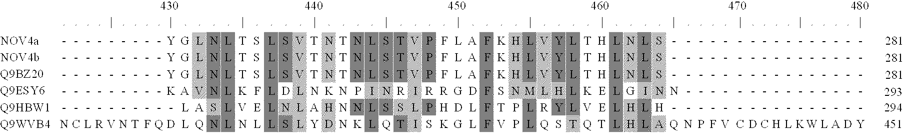

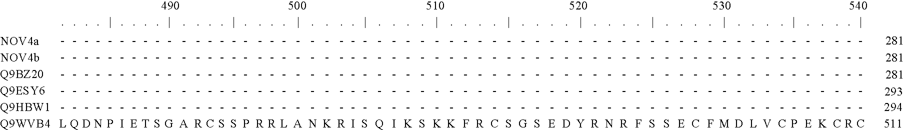

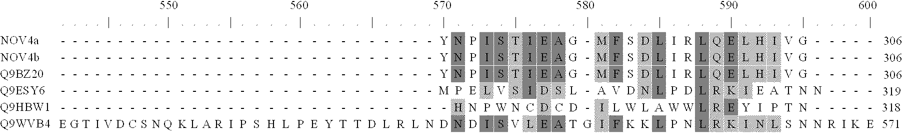

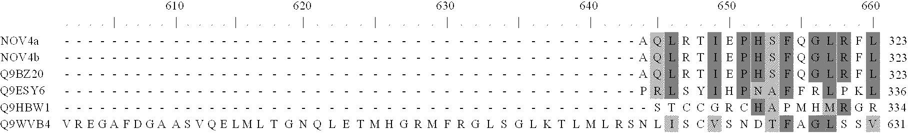

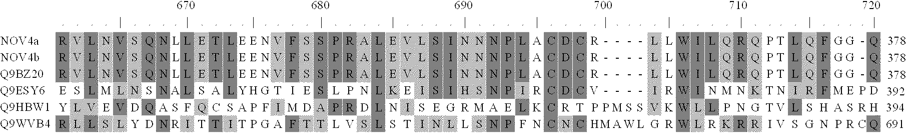

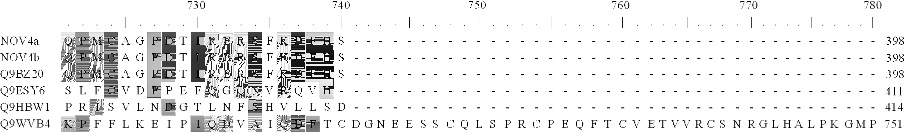

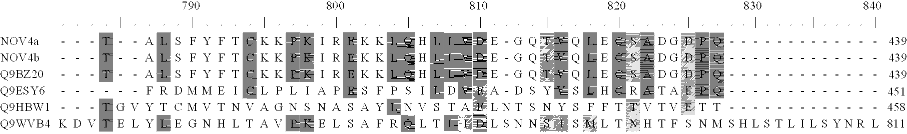

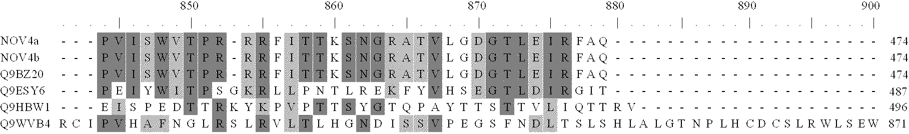

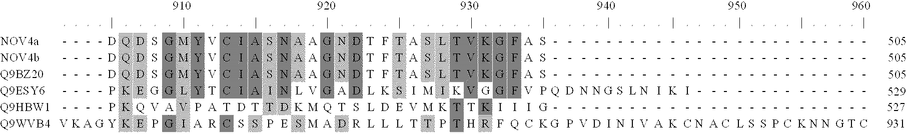

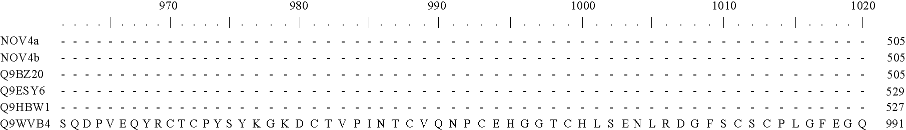

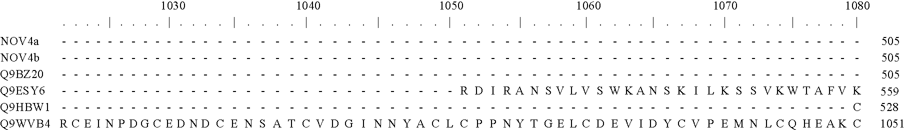

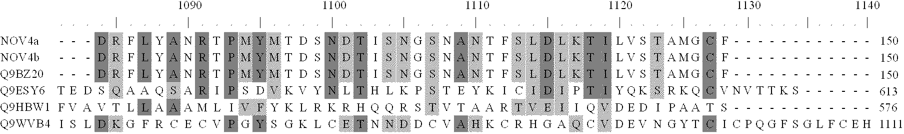

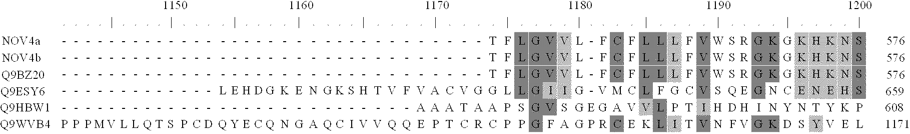

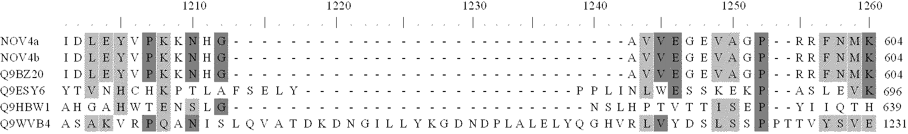

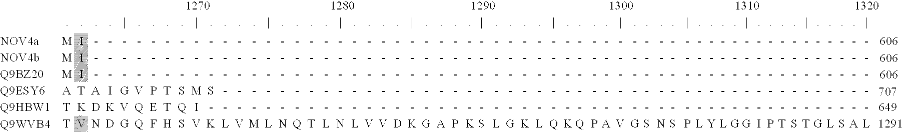

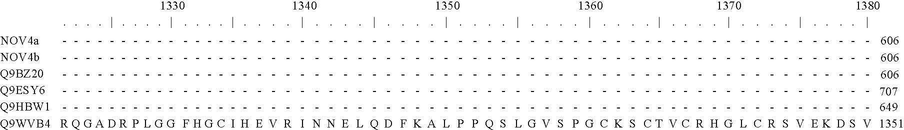

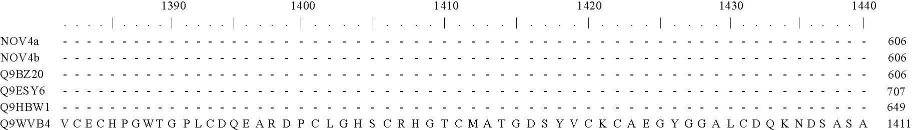

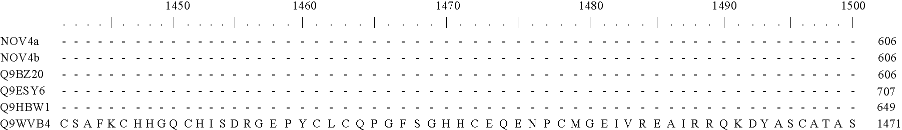

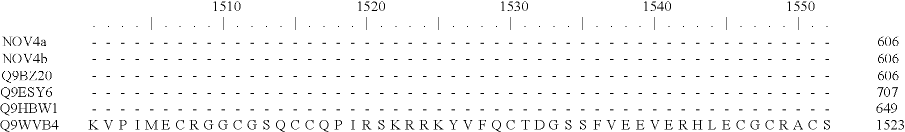

The presence of identifiable domains in the protein disclosed herein was determined by searches using algorithms such as Pfam. Table 4H lists the domain description from DOMAIN analysis results against NOV4.

TABLE 4H
Domain Analysis of NOV 4

| | Region of | | |
|---|---|---|---|
| Model | Homology | Score (bits) | E value |
| Leucine rich | 27-56 | 31.1 | 2.5e−05 |
| repeat N- | | | |
| terminal domain | | | |
| Leucine rich | 58-81 | 9.3 | 45 |
| repeat | | | |
| Leucine rich | 82-105 | 15.8 | 1.1 |
| repeat | | | |

The presence of protein regions in NOV4 that are homologous to a leucine-rich repeat domain is consistent with the identification of NOV4 protein as a Slit-3 -like protein. This indicates that the NOV4 sequence has properties similar to those of other proteins known to contain these domains.

The domain and protein similarity information for the invention suggests that this gene may function as “Slit-3.” As such, the NOV4 protein of the invention may function in the formation and maintenance of the nervous system. NOV4 is implicated, therefore, in disorders involving these tissues.

The nucleic acids and proteins of the invention are useful in potential therapeutic applications implicated in various pathologies/disorders described. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell tpes composing these tissues and cell types derived from these tissues).

The novel nucleic acid encoding the NOV4 of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV4 protein has multiple hydrophilic regions, each of which can be used as an immunogen. The hydropathy plot for invention shows that the protein sequence has an amino terminal hydrophobic region, which could function as a signal peptide to target this sequence to the plasma membrane.

NOV5

A NOV5 polypeptide according to the invention includes a LRR/GPCR-like protein. The nucleic acid sequence (and encoded polypeptide) of two NOV5 sequences - NOV5a and NOV5b are provided.

NOV5a

A NOV5a nucleic acid (also referred to as 133783508ext) of 4245 nucleotides (SEQ ID NO:21) encoding a novel LRR/GPCR-like protein is shown in Table 5A. An open reading frame was identified beginning with an ATG initiation codon at nucleotides 214-216 and ending with a TAA codon at nucleotides 4168-4170. Untranslated regions upstream from the initiation codon and downstream from the termination codon are underlined in Table 5A. The start and stop codons are in bold letters.

TABLE 5A
NOVSa Nucleotide Sequence

(SEQ ID NO:21)

GGGACCCATGCGGCCGTGACCCCCGGCTCCCTAGAGGCCCAGCGCAGCCGCAGCGGACAAAGGAGCATGTCCGCG
CCGGCGAAGGCCCGTCCTCCGGCCGCCATAAGGCTCCGGTCGCCGCTGGGCCCGCGCCGCGCTCCTGCCCGCCCG
GGCTCCGGGGCGGCCCGCTAGGCCAGTGCGCCGCCGCTCGCCCCGCAGGCCCCGCCCCGCAGCATGGAGCCACCC
GGACGCCGGCGGGGCCGCGCGCAGCCGCCGCTGGTGCTGCCGCTCTCGCTGTTAGCGCTGCTCGCGCTGCTGGAA
GCCCGCGGCGCCGGCGGCGCCGCGGCGCTGCCCGCCGGCTGCAAGCACGATCGGCGGCCCCGAGGGGCTGGCAGG
GCGGCGGGCGTGGAGGGCAAGGTGGTGTGCAGCAAGCCTGAACTCGCGCAGGTCGTGCCCCCAGATACTCTGCCC
AACCGCACGGTCACCCTGATTCTGAGTAACAATAAGATATCCGAGCTGAAGAATGGCTCATTTTCTGGGTTAAGT
CTCCTTGAAAGATTGCACCTCCGAAACAATCTTATTAGTAGTATAGATCCAGGTGCCTTCTGGGGACTGTCATCT
CTAAAAAGATTGGATCTGACAAACAATCGAATAGGATGTCTGAATGCAGACATATTTCGAGGACTCACCAATCTG
GTTCGGCTGAACCTTTCGGGGAATTTGTTTTCTTCATTATCTCAAGGAACTTTTGATTATCTTGCGTCATTACGG
TCTTTGGAATTCCAGACTGAGTATCTTTTGTGTGACTGTAACATACTGTGGATGCATCGCTCGGTAAAGGAGAAG
AACATCACGGTACGGGATACCAGGTGTGTTTATCCTAAGTCACTGCAGGCCCAACCAGTCACAGGCGTGAAGCAG
GAGCTGTTGACATGCGGTAAGGGAGAAATCCAAGAATTGCCGTCTTTCTACATGACTCCATCTCATCGCCAAGTT
GTGTTTGAAGGAGACAGCCTTCCTTTCCAGTGCATGGCTTCATATATTGATCAGGACATGCAAGTGTTGTGGTAT
CAGGATGGGAGAATAGTTGAAACCGATGAATCGCAAGGTATTTTTGTTGAAAACAACATGATTCACAACTOCTCC
TTGATTGCCCTAACCATTTCTAATATTCACGCTGGATCTACTGGAAATTGGGGCTGTCATGTCCAGACCAAACGT
GGGAATAATACGAGGACTGTGGATATTGTGGTATTAGAGAGTTCTGCACAGTACTGTCCTCCAQAGAGGGTGGTA
AACAACAAAGGTGACTTCAGATGGCCOAGAACATTGGCAGGCATTACTGCATATCTGCAGTGTACGCGGAACACC
CATGGCAGTGGGATATATCCCGGAAACCCACAGGATGAGAGAAAAGCTTGGCGCAGATGTGATACAGCTGGCTTT
TGGGCAGATGATGATTATTCTCGCTGTCAGTATGCAAATGATGTCACTAGAGTTCTTTATATGTTTATGCCCCTC
AATCTTACCAATGCCGTGGCAACAGCTCGACAGTTACTGGCTTACACTGTGGAAGCAGCCAACTTTTCTGACAAA
ATGGATGTTATATTTGTGGCAGAAATGATTGAAAAATTTGGAAGATTTACCAACGAGGAAAAATCAAAAGAGGTG
ATGGTTGACATTGCAAGTAACATCATGTTGGCTGATGAACGTGTCCTGTGGCTGGCGCAGAGGGAAGCTAAAGCC
TGCAGTAGGATTGTGCAGTGTCTTCAGCGCATTGCTACCTACCGGCTAGCCGGTGGAGCTCACGTTTATTCAACA
TATTCACCCAATATTGCTCTGGAAGCTTATGTCATCAAGTCTACTGGCTTCACGGGGATGACCTGTACCGTGTTC
CAGAAAGTGGCAGCCTCTGATCGTACAGGACTTTCGGATTATGGGACGCCGGATCCAGAGGGAAACCTGGATAAG
CAGCTGAGCTTTAAGTGCAATGTTTCAAATACATTTTCGAGTCTGGCACTAAAGATTGTGGAGGCTTCTATTCAG
CTTCCTCCTTCCCTTTTCTCACCAAAGCAAAAAAGAGAACTCAGACCAACTGATGACTCTCTTTACAAGCTTCAA
CTCATTGCATTCCGCAATCGAAAGCTTTTTCCAGCCACTGGAAATTCAACAAATTTGGCTGATGATGGAAAACGA
CGTACTGTGGTTACCCCTGTGATTCTCACCAAAATAGATGGTGTGAATGTAGATACCCACCACATCCCTGTTAAT
GTGACACTGCGTCGAATTGCACATGGAGCAGATGCTGTTGCAGCCCGGTGCGATTTCGATTTGCTGAACGGACAA
GGAGGCTGGAAGTCAGATGGGTGCCATATACTCTATTCAGATGAAAATATCACTACGATTCAGTGCTACTCCCTT
AGTAACTATGCAGTTTTAATGGATTTGACGGGATCTGAACTATACACCCAGGCGGCCAGCCTCCTGCATCCTGTG
GTTTATACTACCGCTATCATTCTCCTCTTATGTCTCTTAGCCGTCATTGTCAGTTACATATACCATCACAGTTTG
ATTAGAATCAGCCTCAAGAGCTGGCACATGCTTGTGAACTTGTGCTTTCATATTTTCCTAACCTGTGTGGTCTTT
GTGGGAGGAATAACCCAGACTAGGAATGCCAGCATCTGCCAAGCAGTTGGGATAATTCTTCACTATTCCACCCTT
GCCACAGTACTATCGGTAGGAGTGACAGCTCCAAATATCTACAAACAAGTCACTAAAAAAGCTAAAACATGCCAG
GATCCTGATGAACCACCACCTCCACCAAGACCAATGCTCAGGTATCTCATATCTTTGAGATTTTACCTGATTGGT
GGTGGTATCCCCATCATTGTTTGCGGCATAACTGCAGGAGGGAACATTAAGAATTACGGCAGTCGCCCAAACGCA
CCCTGCTGGATGGCATGGGAACCCTCCTTGGGAGCCTTCTATGGGCCAGCCAGCTTCATCACTTTTGTAAACTGC
ATGTACTTTCTGAGCATATTTATTCAGTTGAAAAGACACCCTGAGCGCAAATATGAGCTTAAGGAGCCCACGGAG
GAGCAACAGAGATTGGCAGCCAATGAAAATCGCCAAATAAATCATCAGGATTCAATGTCTTTGTCTCTGATTTCT
ACATCAGCCTTGGAAAATGAGCACACTTTTCATTCTCAGCTCTTGGGGGCCAGCCTTACTTTGCTCTTATATGTT
GCACTGTGGATGTTTGGGGCTTTGGCTGTTTCTTTGTATTACCCTTTGGACTTGGTTTTTAGCTTCGTTTTTGGA
GCCACAAGTTTAAGCTTCAGTGCGTTCTTCGTGGTCCACCATTGTGTTAATAGGGAGGATGTTAGACTTGCGTGG
ATCATGACTTGCTGCCCAGGACGGAGCTCGTATTCAGTCCAAGTCAACGTCCAGCCCCCCAACTCTAATGGGACG
AATGGAGAGGCACCCAAATGCCCCAATAGCAGTGCGGAGTCTTCATGCACAAACAAAAGTGCTTCAAGCTTCAAA
AATTCCTCCCAGGGCTGCAAATTAACAAACTTGCAGGCGGCTGCAGCTCAGTGCCATGCCAATTCTTTACCTTTG
AACTCCACCCCTCAGCTTGATAATAGTCTGACAGAACATTCAATGGACAATGATATTAAAATGCACGTGGCGCCT
TTAGAAGTTCAGTTTCGAACAAATGTGCACTCAAGCCGCCACCATAAAAACAGAAGTAAAGGACACCGGGCAAGC
CGACTCACAGTCCTGAGAGAATATGCCTACGATGTCCCAACGAGCGTGGAA&GAAGCGTGCAGAACGGCTTACCT
AAAAGCCGGCTGGGCAATAACGAAGGACACTCGAGGAGCCGAAGAGCTTATTTAGCCTACAGAGAGAGACAGTAC
AACCCACCCCAGCAAGACAGCAGCGATGCTTGTAGCACACTTCCCAAAAGTAGCACAAATTTTGAAAAGCCAGTT
TCAACCACTAGTAAAAAAGATGCGTTAAGGAAGCCAGCTGTGGTTGAACTTGAAAATCACCAAAAATCTTATGGC
CTCAACTTGGCCATTCAGAATGGACCAATTAAAAGCAATGGGCAGGAGGGACCCTTGCTCGGTACCGATAGCACT
GGCAATGTTAGGACTGGATTATGGAAACACGAAACTACTGTGTAACATTGCTGCGCTTCCTAGGCAGAAATTCAT
ATAAACTGTGATACTCACATTCCTTGAAGCTATGAGCATTTAAAA

In a search of public sequence databases, the NOV5a nucleic acid sequence was located on the p31 region of chromosome 4 has 1326 of 1344 bases (98% identity) with exon 12 of p58 protein kinase (clk-1) gene, mRNA from Homo sapiens (GENBANK-ID: M88565) (E=0.0). Public nucleotide databases include all GenBank databases and the GeneSeq patent database.

The NOV5a protein (SEQ ID NO:22) encoded by SEQ ID NO:21 is 1318 amino acid residues in length and is presented using the one-letter amino acid code in Table 5B.

TABLE 5B
Encoded NOV5a protein sequence

(SEQ ID NO:22)

MEPPGRRRGRAQPPLVLPLSLLALLALLEAGGAGGAAALPAGCKHDGRPRGAGRAAGVEGKVVCS
KPELAQVVPPDTLPNRTVTLILSNNKISELKNGSFSGLSLLERLDLRNNLISSIDPGAFWGLSSL
KRLDLTNNRIGCLNADIFRGLTNLVRLNLSGNLFSSLSQGTFDYLASLRSLEFQTEYLLCDCNIL
WMHRWVKEKNITVRDTRCVYPKSLQAQPVTGVKQELLTCGKGEIQELPSFYMTPSHRQVVFEGDS
LPFQCMASYIDQDMQVLWYQDGRIVETDESQGIFVEKNMIHNCSLIALTISNIQAGSTGNWGCHV
QTKRGNNTRTVDIVVLESSAQYCPPERVVNNKGDFRWPRTLAGITAYLQCTRNTHGSGIYPGNPQ
DERKAWRRCDRGGFWADDDYSRCQYANDVTRVLYMFMPLNLTNAVATARQLLAYTVEAANFSDKM
DVIFVAEMIEKFGRGTKEEKSKEVMVDIASNIMLADERVLWLAQREAKACSRIVQCLQRIATYRL
AGGAHVYSTYSPNIALEAYVIKSTGFTGMTCTVFQKVAASDRTGLSDYGRRDPEGNLDKQLSFKC
NVSNTFSSLALKIVEASIQLPPSLFSPKQKRELRPTDDSLYKLQLIAFRNGKLFPATGNSTNLAD
DGKRRTVVTPVILTKIDGVNVDTHHIPVNVTLRRIAHGADAVAARWDFDLLNGQGGWKSDGCHIL
YSDENITTIQCYSLSNYAVLMDLTGSELYTQAASLLHPVVYTTAIILLLCLLAVIVSYIYHHSLI
RISLKSWHMLVNLCFHIFLTCVVFVGGITQTRNASICQAVGIILHYSTLATVLWVGVTARNIYKQ
VTKKAKRCQDPDEPPPPPRPMLRYLISLFFYLIGGGIPIIVCGITAGGNIKNYGSRPNAPCWMAW
EPSLGAFYGPASFITFVNCMYFLSIFIQLKRHPERKYELKEPTEEQQRLAANENGEINHQDSMSL
SLISTSALENEHTFHSQLLGASLTLLLYVALWMFGALAVSLYYPLDLVFSFVFGATSLSFSAFFV
VHHCVNREDVRLAWIMTCCPGRSSYSVQVNVQPPNSNGTNGEAPKCPNSSAESSCTNKSASSFKN
SSQGCKLTNLQAAAAQCHANSLPLNSTPQLDNSLTEHSMDNDIKMHVAPLEVQFRTNVHSSRHHK
NRSKGHRASRLTVLREYAYDVPTSVEGSVQNGLPKSRLGNNEGHSRSRRAYLAYRERQYNPPQQD
SSDACSTLPKSSRNFEKPVSTTSKKDALRKPAVVELENQQKSYGLNLAIQNGPIKSNGQEGPLLG
TDSTGNVRTGLWKHETTV

NOV5b

A disclosed NOV5b nucleic acid (also referred to as BE304119ext) of 1410 nucleotides (SEQ ID NO:23) encoding a novel LRR/GPCR-like protein is shown in Table 5C. An open reading frame was identified beginning with an AGG initiation codon at nucleotides 204-206 and ending with a TGA codon at nucleotides 1154-1156. Untranslated regions upstream from the initiation codon and downstream from the termination codon are underlined in Table 5C. The start and stop codons are in bold letters.

TABLE 5C
NOV5b Nucleotide Sequence

(SEQ ID NO:23)

TTCAGACGAAACGTGGGAATAACACAAGAACTGTTGACATTGTGGTATTAGAAAGCTCCGCCCAATACTGTCCAC
CAQACAGGGTTGTGAACAACAAAGGTGATTTCAGATGGCCCAGGACGCTGGCGGGCATCACAGCATATCTCCAGT
GTACCCGGAACACCCACAGCAGTGGGATCTACCCCGGAAGCGCACAGGATGAAAGGAAGGCGTGGCGCCCGATGC
GACAGAGGTGACTTTTCGCGCAGATGATCATTATTTCAGGTGCCAGTATGCAAATGACGTCACTAGATTCCTGTA
TATGTTTAATCAGATGCCCCTCAACCTTACAAATGCGGTCGCTACAGCTCGGCAGCTGCTGGCTTACACAGTGGA
GCCCGCCAACTTCTCTGACAAAATGGACGTTATATTTGTGGCTGAAATGATAGAAAAGTTTGGAAGATTTACCAG
AGAGGAAAAATCAAAAGAGCTTGGTGATGTAATGGTCGATGTGGCAAGCAACATCATGTTGGCTGATGAACGGGT
CCTGTGGCTGGCACAGAGGGAAGCGACGGCCTGCAGTCGGATTGTCCAGTGCCTGCAGCGCATTGCCACACATCG
CCTGGCCAGTGGGGCCCACGTGTACTCCACGTACTCGCCCAACATTGCTCTGGAGGCTTACGTCATCAAGGCTGC
TGGCTTCACAGGAATGACCTGCTCCGTGTTCCAGAAGGTGGCTGCCTCCGACCGTGCAGGTCTTTCTGACTATCG
GCGAAGGCACCCCGATGGAAACCTGGATAAGCAGCTGAGCTTCAAATGCAATGTCTCCAGCACCTTCTCAAGCCT
GGCCCTGAAGAACACCATCATGGAGGCCTCCATTCAGCTTCCTTCCTCCCTTTTGTCACCAAAACACAAGCGAGA
AGCCCGAGCGGCGGATGACGCCCTCTAPAAGCTCCAGCTDATTGCCTTCCGCAACGGAAAGCTTTTTCCAGCCAC
TGGAAATTCAACAAAGTTGGCAGACGATGGCAAGCGGCGGACAGTAGTGACCCCTGTGATCCTCACGAAPATAGA
TGGTGCAACCGTAGATACCCACCACATCCCTGTTAATGTGACGCTGCGCCGAATTGCCCACGGAGCACGATGCCG
TTGCTGCGCACGTGGGACTTTGATTTGCTGAACGGCACAACGGAGGCTGGAAGTCACGATTGGGTGCTCGTATAC
TCTACTCCGGATGAGGAACATCACCAGCATTCAGTTGCGGCTCCCTGGGCCACTATGCTGTGGCTATTGGCTCTG
GCTGGGACACATTAGTCCACCCAGCAGGCCAGTCTCTCGCCCTGTGGTTCCCCATTGCATCACATCCCCTCTGGG
TCTTGGAGGATCCCCAGTCATGTCACCCAACTTGGCCGACGCACACAACGCTGCCACCTG

The NOV5b protein (SEQ ID NO:24) encoded by SEQ ID NO:23 is 317 amino acid residues in length and is presented using the one-letter amino acid code in Table 5D.

TABLE 5D
Encoded NOV5b protein sequence

(SEQ ID NO:24)

KEGVAPDATEVAFRADDDYFRCQYANDVTRFLYMFNQMPLNLTNAVATARQLLAYTVEPANFSDK
MDVIFVAEMIEKFGRFTREEKSKELGDVMVDVASNIMLADERVLWLAQREAKACSRIVQCLQRIA
THRLASGAHVYSTYSPNIALEAYVIKAAGFTGMTCSVFQKVAASDRAGLSDYGRRDPDGNLDKQL
SFKCNVSSTFSSLALKNTIMEASIQLPSSLLSPKHKREARAADDALYKLQLIAFRNGKLFPATGN
STKLADDGKRRTVVTPVILTKIDGATVDTHHIPVNVTLRRIAHGARCGCCARGTLIC

NOV5 Clones

The Psort profile for NOV5a predicts that this sequence has a signal sequence and is likely to be localized at the plasma membrane with a certainty of 0.6400. In other embodiments, NOV5a localizes to the Golgi body with a certainty of 0.4600, the endoplasmic reticulum (membrane) with a certainty of 0.3700, and the endoplasmic reticulum (lumen) with a certainty of 0.1000. The most likely cleavage site for a NOV5a peptide is between amino acids 38 and 39, at: AAA-LP.

A search against the Patp database, a proprietary database that contains sequences published in patents and patent publications, yielded several homologous proteins to NOV5a shown in Table 5E.

TABLE 5E
Patp Results for NOV 5a

| | | Smallest | |
|---|---|---|---|
| | | Sum | |
| | High | Prob | |
| Sequences producing High-scoring Segment Pairs: | Score | P | (N) |
| patp:Y99347 Human PRO1113 (UNQ556) amino acid sequence S... | 2998 | 1.1e−312 | 1 |
| patp:W27161 Mouse receptor ME2 - Mus musculus, 2707 aa. | 283 | 3.1e−23 | 2 |
| patp:W27160 Mouse receptor ME2 region comprising ME2 (22) ... | 283 | 3.8e−23 | 3 |
| patp:Y13393 Amino acid sequence of protein PR0335 - Homo ... | 299 | 5.3e−21 | 2 |
| patp:Y70672 Human PR0335 protein - Homo sapiens, 1059 aa. | 299 | 5.3e−21 | 2 |
| patp:Y08095 Human PR0335 protein - Homo sapiens, 1059 aa. | 299 | 5.3e−21 | 2 |
| patp:Y70674 Human PR0326 protein - Homo sapiens, 1119 aa. | 299 | 6.3e−21 | 2 |
| patp:Y08114 Human PR0326 protein - Homo sapiens, 1119 aa. | 299 | 6.3e−21 | 2 |
| patp:Y13395 Amino acid sequence of protein PR0326 - Homo ... | 299 | 6.3e−21 | 2 |

In a BLAST search of public sequence databases, it was found, for example, that the full amino acid sequence of NOV5a was found to have 315 of 554 amino acid residues (98%) identical to, and 404 of 554 amino acid residues (99%) similar to, the KIAA1531 PROTEIN of 1060 amino acid residue LRR/GPCR-like protein from Homo sapiens (GENBANK-ID:BAA96055) (E=4.1e⁻¹⁸⁵).

NOV5a also has homology to the proteins shown in the BLASTP data in Table 5F.

TABLE 5F
BLAST results for NOV5a

| Gene Index/ | | Length | Identity | Positives | |
|---|---|---|---|---|---|
| Identifier | Protein/ Organism | (aa) | (%) | (%) | Expect |
| SPTREMBL-ACC:Q9P1Z7 | KIAA1531 PROTEIN | 1060 | 316/654 | 404/654 | 5.5 e − |
| | [Homo sapiens] | | (48%) | (61%) | 185 |
| TREMBLNEW-ACC:BAB47457 | KIAA1828 PROTEIN | 496 | 174/394 | 232/394 | 1.0 e − |
| | [Homo sapiens] | | (44%) | (58%) | 74 |

Similar BLAST analysis of NOV5b revealed that this polypeptide has homology to the proteins shown in the BLASTP data in Table 5G.

TABLE 5G
BLAST results for NOV5b

| Gene Index/ | | Length | Identity | Positives | |
|---|---|---|---|---|---|
| Identifier | Protein/ Organism | (aa) | (%) | (%) | Expect |
| SPTREMBL-ACC:QP1Z7 | KIAA1531 protein | 1060 | 134/294 | 174/294 | 4.5 e − |
| | (fragment) | | (45%) | (59%) | 53 |
| | [Homo sapiens] | | | | |
| STREMBL-ACC:Q9VYF1 | CG15744 protein | 1797 | 58/235 | 111/235 | 0.00045 |
| | [Drosophila | | (25%) | (47%) | |
| | melanogaster] | | | | |

A multiple sequence alignment is given in Table 5H, with the NOV5a and NOV5b shown on line 1 and line 2, respectively. This Clustal W analysis compares the NOV5 protein with the related protein sequences shown in Tables 5F and 5G. The homologies shared by NOV5a and NOV5b polypeptides are also shown in Table 5H.

[image]

[image]

[image]

[image]

[image]

[image]

[image]

[image]

The presence of identifiable domains in the protein disclosed herein was determined by searches using algorithms such as Pfam. Table 5I lists the domain description from DOMAIN analysis results against NOV5a.

TABLE 5I
Domain Analysis of NOV 5a

| Model | Score (bits) | E value |
|---|---|---|
| Leucine rich | 79.9 | 5.4e−20 |
| repeat | | |
| Leucine rich | 41.7 | 1.6e−08 |
| repeat C- | | |
| terminal domain | | |
| Latrophilin/CL- | 25.4 | 0.0012 |
| 1-like GPS | | |
| domain | | |
| Immunoglobulin | 21.7 | 3.6e−05 |
| domain | | |
| Hormone receptor | 6.8 | 0.069 |
| domain | | |
| 7 transmembrane | 46.2 | 3.6e−05 |
| receptor | | |
| (Secretin | | |
| family) | | |

The presence of protein regions in NOV5a that are homologous to a leucine-rich repeat domain is consistent with the identification of NOV5 protein as a LRR/GPCR-like protein. This indicates that the NOV5 sequence has properties similar to those of other proteins known to contain these domains.

The domain and protein similarity information for the invention suggests that this gene may function as “LRR/GPCR”. As such, the NOV5 protein of the invention may function in the formation and maintenance of the nervous system. NOV5 is implicated, therefore, in disorders involving these tissues, such as, for example, abnormal angiogenesis, like cancer and more specifically aggressive, metastatic cancer, more specifically tumor of the lung, kidney, brain, liver and colon.

The nucleic acids and proteins of the invention are useful in potential therapeutic applications implicated in various pathologies/disorders described. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV5a protein has multiple hydrophilic regions, each of which can be used as an immunogen. In one embodiment, a contemplated NOV5a epitope is from about amino acids 20 to 30. In another embodiment, a NOV5a epitope is from about amino acids 50 to 75. In additional embodiments, NOV5a epitopes are from about amino acids 100 to 120, from about 180 to 300, from about amino acids 325 to 425, from about amino acids 525 to 600, from about amino acids 625 to 725, from about amino acids 850 to 900, from about amino acids 950 to 1000, and from about amino acids 1050 to 1350. These novel proteins can be used in assay systems for functional analysis of various human disorders, which are useful in understanding of pathology of the disease and development of new drug targets for various disorders.

NOV6

A disclosed NOV6 nucleic acid (also referred to as jgigc_draft_citb-el_{—}2540b15_{—}20000803_da1) of 961 nucleotides (SEQ ID NO:25) encoding a novel Major Histocompatibility Complex Enhancer-Binding Protein MAD3-like protein is shown in Table 6A. An open reading frame was identified beginning with an ATG initiation codon at nucleotides 1-3 and ending with a TGA codon at nucleotides 955-957. Untranslated regions upstream from the initiation codon and downstream from the termination codon are underlined in Table 6A. The start and stop codons are in bold letters.

TABLE 6A
NOV6 Nucleotide Sequence

(SEQ ID NO:25)

ATGTTCCAGGCGGCCGAGCGCCCCCAGGAGTGGGCCATGGAGGGCCCCCGCGACGGGCTGAAGAAGGAGCGGCTA
CTGGACGACCGCCACGACAGCGGCCTGGACTCCATGAAAGACGAGGCTCATCATCGCTGGCCTCCAGAAACCCCG
GCCTTGCGCAATCCCCCGCAGCACGCGCCTCCCTGGGCCCCACGCGGTGCACTCACCACCCCTGGGGTTTTTCCC
TCTCTTCCCCACAGGTTCCTGCACTTGGCCATCATCCATGAAGAAAAGGCACTGACCATGGAAGTGATCCGCCAG
GTGAAGGGAGACCTGGCCTTCCTCAACTTCCAGAACAACCTGCAGCAGACTCCACTCCACTTGGCTGTGATCACC
AACCAGCCAGAAATTGCTGAGGCACTTCTGGGAGCTGGCTGTGATCCTGAGCTCCGAGACTTTCGAGGAAATACC
CCCCTACACCTTGCCTGTGAGCAGGGCTGCCTGGCCAGCGTGGGAGTCCTGACTCAGTCCTGCACCACCCCGCAC
CTCCACTCCATCCTGAAGGCTACCAACTACAATGGCCACACGTGTCTACACTTAGCCTCTATCCATGGCTACCTG
GGCATCGTGGAGCTTTTGGTGTCCTTGGGTGCTGATGTCAATGCTCAGGAGCCCTGTAATGGCCGGACTGCCCTT
CACCTCGCAGTGGACCTGCAAAATCCTGACCTGGTGTCGCTCCTGTTGAAGTGTGGGGCTGATGTCAACAGAGTT
ACCTACCAGGGCTATTCTCCCTACCAGCTCACCTGGGGCCGCCCAAGCACCCGGATACAGCAGCAGCTGGGCCAG
CTGACACTAGAAAACCTTCAGATGCTGCCAGAGAGTGAGGATGAGGAGAGCTATGACACAGAGTCAGAGTTCACG
GAGTTCACAGAGGACGAGCTGCCCTATGATGACTGTGTGTTTGGAGGCCAGCGATGATGAG

Variant sequences of NOV6 are included in Example 2, Table 54. A variant sequence can include a single nucleotide polymorphism (SNP). A SNP can, in some instances, be referred to as a “cSNP” to denote that the nucleotide sequence containing the SNP originates as a cDNA.

In a search of public sequence databases, the NOV6 nucleic acid sequence, located on chromosome 4 has 1326 of 1344 bases (98% identity) with exon 12 of p58 protein kinase (clk-1) gene, mRNA from Homo sapiens (GENBANK-ID: M88565) (E=0.0). Public nucleotide databases include all GenBank databases and the GeneSeq patent database.

The NOV6 protein (SEQ ID NO:26) encoded by SEQ ID NO:25 is 318 amino acid residues in length has a molecular weight of 35427.5 Daltons and is presented using the one-letter amino acid code in Table 6B. The Psort profile for NOV6 predicts that this sequence has no signal sequence and is likely to be localized at the cytoplasm with a certainty of 0.6500. In other embodiments, the NOV6 protein localizes to the lysosome (lumen) with a certainty of 0.2195, or the mitochondrial membrane space with a certainty of 0.1000.

TABLE 6B
Encoded NOV6 protein sequence

(SEQ ID NO:26)

MFQAAERPQEWAMEGPRDGLKKERLLDDRHDSGLDSMKDEAHHRWPPETPALRNPPQHAPPWAPRGALTTPGV
FPSLPHRFLHLAIIHEEKALTMEVIRQVKGDLAFLNFQNNLQQTPLHLAVITNQPEIAEALLGAGCDPELRDF
RGNTPLHLACEQGCLASVGVLTQSCTTPHLHSILKATNYNGHTCLHLASIHGYLGIVELLVSLGADVNAQEPC
NGRTALHLAVDLQNPDLVSLLLKCGADVNRVTYQGYSPYQLTWGRPSTRIQQQLGQLTLENLQMLPESEDEES
YDTESEFTEFTEDELPYDDCVFGGQR

In a BLAST search of public sequence databases, it was found, for example, that the full amino acid sequence of NOV6 was found to have 288 of 318 amino acid residues (90%) identical to, and 292 of 318 amino acid residues (91%) similar to, the MAJOR HISTOCOMPATIBILITY COMPLEX ENHANCER-BINDING PROTEIN of 1060 amino acid residue LRR/GPCR-like protein from Homo sapiens (SWISSPROT-ACC:P25963) (E=5.5e⁻¹⁵¹).

NOV6 has homology to the proteins shown in the BLASTP data in Table 6C.

TABLE 6C
BLAST results for NOV6

| Gene Index/ | | Length | Identity | Positives | |
|---|---|---|---|---|---|
| Identifier | Protein/ Organism | (aa) | (%) | (%) | Expect |
| MAD3₋ | Major | 317 | 267/322 | 271/322 | 1 e − |
| HUMAN | histocompatibility | | (83%) | (84%) | 150 |
| | complex | | | | |
| | enhancer-binding | | | | |
| | protein mad3 | | | | |
| | (nuclear factor | | | | |
| | kappa-b | | | | |
| | inhibitor) (i- | | | | |
| | kappa-b-alpha) | | | | |
| | (ikba) | | | | |
| | [Homo sapiens] | | | | |
| Q08353 | ECI-6/IKBA | 314 | 256/322 | 262/322 | 1 e − |
| | protein | | (80%) | (81%) | 139 |
| | [Sus scrofa] | | | | |
| Q63746 | RL/IF-1 mRNA | 314 | 249/322 | 259/322 | 1 e − |
| | [Rattus | | (77%) | (80%) | 136 |
| | norvegicus] | | | | |
| Q9Z1E3 | i KAPPA b alpha | 238 | 203/238 | 207/238 | 1 e − |
| | (fragment) | | (85%) | (87%) | 114 |
| | [Mus musculus] | | | | |
| Q91974 | Rel-associated | 318 | 198/321 | 223/321 | 8 e − |
| | pp40 | | (62%) | (69%) | 98 |
| | [gallus gallus] | | | | |

A multiple sequence alignment is given in Table 6D, with the NOV6 protein being shown on line 1 in Table 6D in a ClustalW analysis, and comparing the NOV6 protein with the related protein sequences shown in Table 6C. This BLASTP data is displayed graphically in the ClustalW in Table 6D.

TABLE 6D
ClustalW Analysis of NOV6

1. >NOV6; SEQ ID NO:26
2. >MAD3_HUMAN/Major histocompatibility complex enhancer-binding protein mad3 (nuclear
factor kappa-b inhibitor) (i-kappa-b-alpha) (ikba)[Homo sapiens]; SEQ ID NO:49
3. >Q08353/ECI-6/IKBA protein[Sus scrofa]; SEQ ID NO:50
4. >Q63746/RL/IF-1 mRNA [Rattus norvegicus]; SEQ ID NO:51
5. >Q9Z1E3/i KAPPA b alpha (fragment)[Mus musculus]; SEQ ID NO:52
6. >Q91974/Rel-associated pp40 [gallus gallus]: SEQ ID NO:53

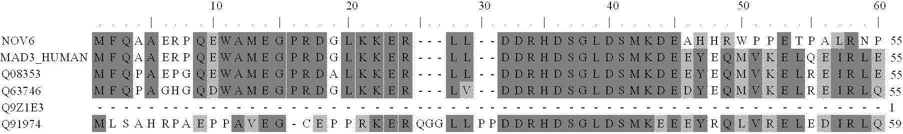

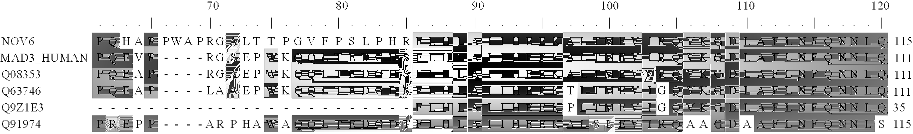

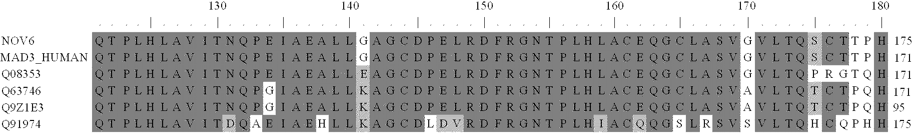

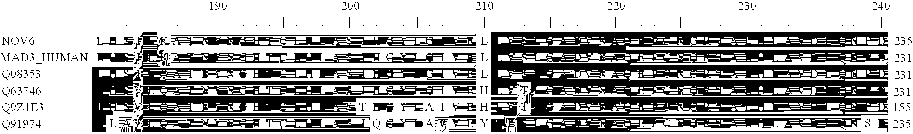

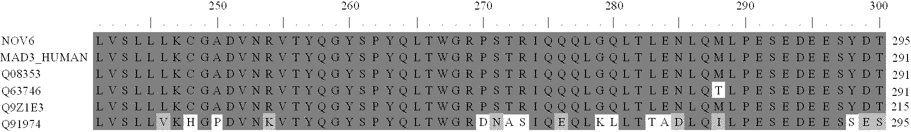

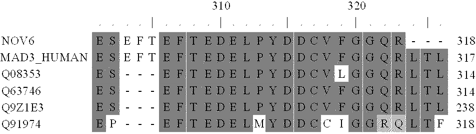

The presence of identifiable domains in the protein disclosed herein was determined by searches using algorithms such as Pfam. Table 6E lists the domain description from DOMAIN analysis results against NOV6.

TABLE 6E
Domain Analysis of NOV6

| Model | Score (bits) | E value |
|---|---|---|
| Ank repeat | 138.6 | 1.1e-37 |

The presence of protein regions in NOV6 that are homologous to a leucine-rich repeat domain is consistent with the identification of NOV6 protein as a Major Histocompatibility Complex Enhancer-Binding Protein MAD3-like protein. This indicates that the NOV6 sequence has properties similar to those of other proteins known to contain these domains.

The domain and protein similarity information for the invention suggests that this gene may function as “Major Histocompatibility Complex Enhancer-Binding Protein MAD3”. As such, the NOV6 protein of the invention may function in the formation and maintenance of the immune system. NOV6 is implicated, therefore, in disorders involving these tissues.

The nucleic acids and proteins of the invention are useful in potential therapeutic applications implicated in various pathologies/disorders described. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV6 protein has multiple hydrophilic regions, each of which can be used as an immunogen. In one embodiment, a contemplated NOV6 epitope is from about amino acids 1 to 75. In another embodiment, a NOV6 epitope is from about amino acids 125 to 160. In additional embodiments, NOV6 epitopes are from about amino acids 175 to 190, from about 200 to 230, and from about amino acids 240 to 320. These novel proteins can be used in assay systems for functional analysis of various human disorders, which are useful in understanding of pathology of the disease and development of new drug targets for various disorders.

NOV7

A disclosed NOV7 nucleic acid (also referred to as GMAP001948_A) of 457 nucleotides (SEQ ID NO:27) encoding a novel Interleukin-9-like protein is shown in Table 7A. An open reading frame was identified beginning with no initiation codon and ending with a TAA codon at nucleotides 445-447. Untranslated regions upstream from the initiation codon and downstream from the termination codon are underlined in Table 7A. The start and stop codons are in bold letters.

TABLE 7A
NOV7 Nucleotide Sequence

(SEQ ID NO:27)

CATGATTGTGTTAGAAAATCTGAAATTACTAGAACTAATAAGTTATTTAACGAGTTTGTACAAGATCAATATTCA
AAACTCCATTGTATTCAATACATTAGCAATGAAAAAATAGATATCCATACTCAATTCACTTTGCCTTTCACCACT
CTCTGCCATCTCTGTCTGACGCTCCCATGCCCAGAATGTTCTTTGTGTTGTTTCTCCTTCATAGAAGCCTTATTT
TCAGTAACCTCAAACTGTAAACAATCCAAATATCCATTAACCAGGTATAAAGAAATTTATTCCATATTGAAAAAG
GGGGTTGTTAGTTCAAAGGAACAGAAAAATCTTAAATGTCCATTTTTATCCTGTGAACAGCCATGCAACCAAACT
GCAGCAAGCAACATACTGATATTTCTGAAGAGTCTCCTGGAAATTTGCCAGGAAGAAAAGATGAGAGATTAAGAA
GCAGAGT

In a search of public sequence databases, the NOV7 nucleic acid sequence, located on the p31 region of chromosome 4 has 152 of 214 bases (71%) identical to a hp40 gene for P40 cytokine mRNA from Homo sapiens (GENBANK-ID: X17543) (E=9.5e⁻¹⁴). Public nucleotide databases include all GenBank databases and the GeneSeq patent database.

The NOV7 protein (SEQ ID NO:28) encoded by SEQ ID NO:27 is 148 amino acid residues in length and is presented using the one-letter amino acid code in Table 7B. The Psort profile for NOV7 predicts that this sequence has no signal sequence and is likely to be localized at the cytoplasm with a certainty of 0.4500. In other embodiments, the NOV7 protein localizes to the microbody (peroxisome) with a certainty of 0.3000, the mitochondrial matrix space with a certainty of 0.1000, or the lysosome (lumen) with a certainty of 0.1000. The most likely cleavage site for a NOV7 peptide is between amino acids 66 and 67, at SLC-CF.

TABLE 7B
Encoded NOV7 protein sequence

(SEQ ID NO:28)

HDCVRKSEITRTNKLFNEFVQDQYSKLHCIQYISNEKIDIHTQFTLPFTTLCHLCLTLPCPECSLCCFSFIEA
LFSVTSNCKQSKYPLTRYKEIYSILKKGVVSSKEQKNLKCPFLSCEQPCNQTAASNILIFLKSLLEICQEEKM
RD

In a BLAST search of public sequence databases, it was found, for example, that the full amino acid sequence of NOV7 was found to have 52 of 98 amino acid residues (53%) identical to, and 63 of 98 amino acid residues (64%) similar to, the 144 amino acid residue INTERLEUKIN-9 PRECURSOR (IL-9) (T-CELL GROWTH FACTOR P40) (P40 CYTOKINE) protein from Homo sapiens (P15248) (E=6.5e⁻²¹).

NOV7 has homology to the proteins shown in the BLASTP data in Table 7C.

TABLE 7C
BLAST results for NOV7

| Gene Index/ | Protein/ | Length | Identity | Positives | |
|---|---|---|---|---|---|
| Identifier | Organism | (aa) | (%) | (%) | Expect |
| ptnr:SWISSNEW- | Interleukin-9 | 144 | 52/98 | 63/98 | 1.5 e − |
| ACC:P15248 | precursor (IL-9) | | (53%) | (64%) | 21 |
| | (T-cell growth | | | | |
| | factor P40) (P40 | | | | |
| | cytokine) | | | | |
| | [Homo sapiens] | | | | |
| ptnr:SWISSPROT- | INTERLEUKIN-9 | 144 | 40/98 | 57/98 | 4.2 e − |
| ACC:P15247 | PRECURSOR | | (40%) | (58%) | 11 |
| | (IL-9) (T-CELL GROWTH | | | | |
| | FACTOR P40) (P40 | | | | |
| | CYTOKINE) | | | | |
| | [Mus musculus] | | | | |

A multiple sequence alignment is given in Table 7D, with the NOV7 protein being shown on line 1 in Table 7D in a ClustalW analysis, and comparing the NOV7 protein with the related protein sequences shown in Table 7C. This BLASTP data is displayed graphically in the ClustalW in Table 7D.

TABLE 7D
ClustalW Analysis of NOV7

1. >NOV7; SEQ ID NO:28
2. >P15248/Interleukin-9 (IL-9) [Homo sapiens]; SEQ ID NO:54
3. >P15247/Interleukin-9 Precursor (IL-9) [Mus musculus]; SEQ ID NO:55

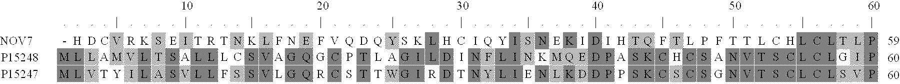

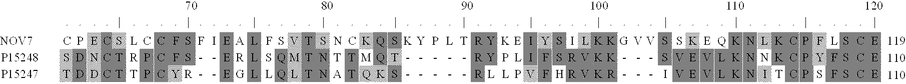

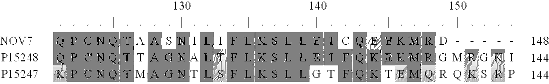

The presence of identifiable domains in the protein disclosed herein was determined by searches using algorithms such as Pfam. Table 7E lists the domain description from DOMAIN analysis results against NOV7.

TABLE 7E
Domain Analysis of NOV7

| Model | Score (bits) | E value |
|---|---|---|
| IL7t | 4 | 0.099 |

The presence of protein regions in NOV7 that are homologous to a leucine-rich repeat domain is consistent with the identification of NOV7 protein as a Interleukin-9-like protein. This indicates that the NOV7 sequence has properties similar to those of other proteins known to contain these domains.

The domain and protein similarity information for the invention suggests that this gene may function as “Interleukin-9”. As such, the NOV7 protein of the invention may function in asthma, various types of cancer, azoospermia, learning disabilities, and facial dysmorphism, multiple sclerosis, autoimmune encephalomyelitis, X-linked severe combined immunodeficiency and other immunological disorders.

The nucleic acids and proteins of the invention are useful in potential therapeutic applications implicated in various pathologies/disorders described. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV7 protein has multiple hydrophilic regions, each of which can be used as an immunogen. In one embodiment, a contemplated NOV7 epitope is from about amino acids 5 to 45. In another embodiment, a NOV7 epitope is from about amino acids 70 to 125. These novel proteins can be used in assay systems for functional analysis of various human disorders, which are useful in understanding of pathology of the disease and development of new drug targets for various disorders.

NOV8

A disclosed NOV8 nucleic acid (also referred to as .SC129285515_A) of 1155 nucleotides (SEQ ID NO:29) encoding a novel 5-Hydroxytyptamine receptor-like protein is shown in Table 8A. An open reading frame was identified beginning with an ATG initiation codon at nucleotides 5-7 and ending with a TGA codon at nucleotides 1145-1147. Untranslated regions upstream from the initiation codon and downstream from the termination codon are underlined in Table 8A. The start and stop codons are in bold letters.

TABLE 5A
NOV8 Nucleotide Sequence

(SEQ ID NO:29)

CGCCATGGAGGCCGCTAGCCTTTCAGTGGCCACCGCCGGCGTTGCCCTTGCCCCCGAGACCAGCAGCCCGGCGTT
GCCCTTGCCCTGGGACCCGAGACCAGCAGCAGGACCCGGGACCCCAAGCCCGAGAGGGATACTCGGTTCGACCCC
GACCGGCGCCGTCCTGCCGGGCCGAGGGCCGCCCTTCTCTGTCTTCACGGTCCTGGTGGTGACGCTGCTAGTGCT
GCTGATCGCTGCCACTTTCCTGTGGAACCTGCTGGTTCCGGTCACCATCCCGCGGGTCCGTGCCTTCCACCGCGT
GCCGCATAACTTGGTGGCCTCGACGGCCGTCTCGGACGAACTAGTGGCAGCGCTGGCGATGCCACCGAGCCTGGC
GAGTGAGCTGTCGACCGGGCGACGTCGGCTGCTGGGCCGCCACGTGTGGATCTCCTTCGACGCCCTGTGCTGCCC
CGCCGGCCTCGGGAACGTGGCGGCCATCGCCCTGGGCCGCGACGGGGCCATCACACGGCACCTGCAGCACACGCT
GCGCACCCGCAGCCGCGCCTCGTTGCTCATGATCGCGCTCGCCCGGGTGCCGTCGGCGCTCATCGCCCTCGCGCC
GCTGCTCTTTGGCCGGGGCGAGGTGTGCGACGCTCGGCTCCAGCGCTGCCAGGTGAGCCGGGAACCCTCCTATGC
CGCCTTCTCCACCCGCGGCGCCTTCCACCTGCCGCTTGGCGTGGTGCCGTTTGTCTACCGGAAGATCTACGAGGC
GGCCAAGTTTCGTTTCGGCCGCCGCCGGAGAGCTGTGCTGCCGTTGCCGGCCACCATGCAGGTGAAGGAAGCACC
TGATGAGGCTGAAGTGGTGTTCACGGCACATTGCAAAGCAACGGTGTCCTTCCAGGTGAGCGGGGACTCCTGGCG
GGAGCAGAAGGAGAGGCGAGCAGCCATGATGGTGGGAATTCTGATTGGCGTGTTTGTGCTGTGCTGGATCCCCTT
CTTCCTGACGGAACTCATCAGCCCACTCTGTGCCTGCAGCCTGCCCCCCATCTGGAAAAGCATATTTCTGTGGCT
TGGCTACTCCAATTCTTTCTTCAACCCCCTGATTTACACAGCTTTTAACAAGAACTACAACAATGCCTTCAAGAG
CCTCTTTACTAAGCAGAGATGAACACAGGG

In a search of public sequence databases, the NOV8 nucleic acid sequence, located on the p31 region of chromsome 2 has 812 of 1089 bases 74%) is identical to a 5-HT5B serotonin receptor mRNA from Mus musculus (GENBANK-ID: X69867) (E=1.8e⁻¹¹⁵). Public nucleotide databases include all GenBank databases and the GeneSeq patent database.

The NOV8 protein (SEQ ID NO:30) encoded by SEQ ID NO:29 is 380 amino acid residues in length and is presented using the one-letter amino acid code in Table 8B. The Psort profile for NOV8 predicts that this sequence has a signal sequence and is likely to be localized at the endoplasmic reticulum (membrane) with a certainty of 0.6850. In other embodiments, the NOV8 protein localizes to the plasma membrane with a certainty of 0.6400, the Golgi body with a certainty of 0.4600, or the endoplasmic reticulum (lumen) with a certainty of 0.1000. The most likely cleavage site for a NOV8 peptide is between amino acids 16 and 17, at ALA-PE.

TABLE 8B
Encoded NOV8 protein sequence

(SEQ ID NO:30)

MEAASLSVATAGVALAPETSSPALPLPWDPRPAAGPGTPSPRGILGSTPSGAVLPGRGPPFSVFTVLVVTLLV
LLIAATFLWNLLVPVTIPRVRAFHRVPHNLVASTAVSDELVAALAMPPSLASELSTGRRRLLGRHVWISFDAL
CCPAGLGNVAAIALGRDGAITRHLQHTLRTRSRASLLMIALARVPSALIALAPLLFGRGEVCDARLQRCQVSR
EPSYAAFSTRGAFHLPLGVVPFVYRKIYEAAKFRFGRRRRAVLPLPATMQVKEAPDEAEVVFTAHCKATVSFQ
VSGDSWREQKERRAAMMVGILIGVFVLCWIPFFLTELISPLCACSLPPIWKSIFLWLGYSNSFFNPLIYTAFN
KNYNNAFKSLFTKQR

In a BLAST search of public sequence databases, it was found, for example, that the full amino acid sequence of NOV8 was found to have 288 of 362 amino acid residues (79%) identical to, and 306 of 362 amino acid residues (84%) similar to, the 370 amino acid residue 5-HYDROXYTRYPTAMINE 5B RECEPTOR (5-HT-5B) (SEROTONIN RECEPTOR) protein from Mus musculus (ACC:P31387) (E=2.8e⁻¹⁴⁴).

NOV8 has homology to the proteins shown in the BLASTP data in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| 5H5B₋RAT | 5- | 370 | 271/383 | 292/383, | 1 e − |
| | hydroxytrypt- | | (71%) | (76%) | 145 |
| | amine 5b receptor | | | | |
| | (5-ht-5b) | | | | |
| | (serotonin re- | | | | |
| | ceptor) mr22) | | | | |
| | [Rattus | | | | |
| | norvegicus] | | | | |
| 5H5B₋ | 5- | 370 | 273/383 | 293/383, | 1 e − |
| MOUSE | hydroxytrypt- | | (71%) | (77%) | 145 |
| | amine 5b receptor | | | | |
| | (5-ht-5b) | | | | |
| | (serotonin recep- | | | | |
| | tor) | | | | |
| | [Mus musculus] | | | | |
| 5H5A₋ | 5- | 357 | 210/346 | 244/346, | 1 e − |
| HUMAN | hydroxytrypt- | | (61%) | (71%) | 106 |
| | amine 5a receptor | | | | |
| | (5-ht-5a) | | | | |
| | (serotonin recep- | | | | |
| | tor) (5-ht-5) | | | | |
| | [Homo sapiens] | | | | |
| 5H5A₋RAT | 5- | 357 | 195/308 | 229/308, | 1 e − |
| | hydroxytrypt- | | (63%) | (74%) | 102 |
| | amine 5a receptor | | | | |
| | (5-ht-5a) | | | | |
| | (serotonin re- | | | | |
| | ceptor) (rec17) | | | | |
| | [Rattus | | | | |
| | norvegicus] | | | | |
| 5H5A₋ | 5- | 357 | 195/308 | 228/308, | 1 e − |
| MOUSE | hydroxytrypt- | | (63%) | (74%) | 101 |
| | amine 5a receptor | | | | |
| | (5-ht-5a) | | | | |
| | (serotonin recep- | | | | |
| | tor) (5-ht-5) | | | | |
| | [Mus musculus] | | | | |

A multiple sequence alignment is given in Table 8D, with the NOV8 protein being shown on line 1 in Table 8D in a ClustalW analysis, and comparing the NOV8 protein with the related protein sequences shown in Table 8C. This BLASTP data is displayed graphically in the ClustalW in Table 8D.

TABLE 8D
ClustalW Analysis of NOV8

1. >NOV8; SEQ ID NO:30
2. >5H5B_RAT/5-hydroxytryptamine 5b receptor [Rattus norvegicus]; SEQ ID NO:56
3. >5H5B_MOUSE/5-hydroxytryptamine 5b receptor [Mus musculus]; SEQ ID NO:57
4. >5H5A_HUMAN/5-hydroxytryptamine 5a recptor [Homo sapiens]; SEQ ID NO:58
5. >5H5A_RAT/5-hydroxytryptamine 5a receptor [Rattus norvegicus]; SEQ ID NO:59
6. >5H5A_MOUSE/5-hydroxytryptamine 5a receptor [Mus musculus]; SEQ ID NO:60

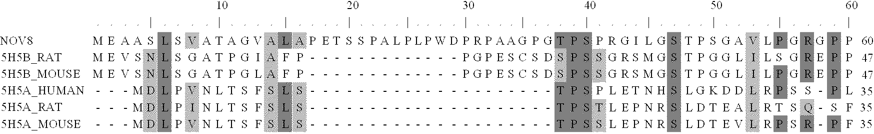

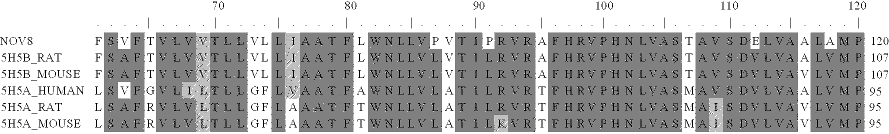

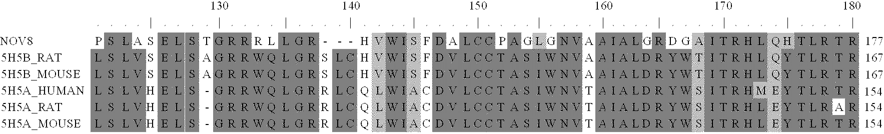

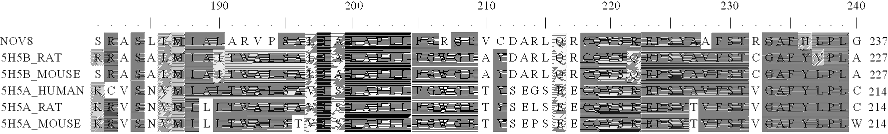

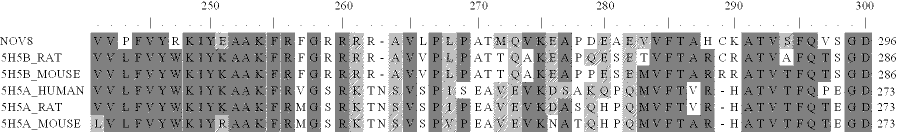

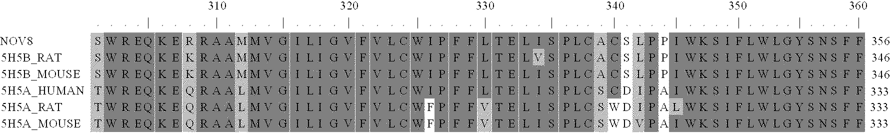

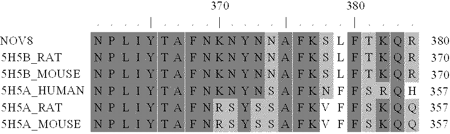

The presence of identifiable domains in the protein disclosed herein was determined by searches using algorithms such as Pfam. Table 8E lists the domain description from DOMAIN analysis results against NOV8.

TABLE 8E
Domain Analysis of NOV8

| | | Score | |
|---|---|---|---|
| Model | Range | (bits) | E value |
| 7tm_1, 7 | 83-361 | 120 | 1e-28 |
| transmembrane | | | |
| receptor | | | |
| (rhodopsin | | | |
| family) | | | |

The presence of protein regions in NOV8 that are homologous to a leucine-rich repeat domain is consistent with the identification of NOV8 protein as a 5-Hydroxytryptamine receptor-like protein. This indicates that the NOV8 sequence has properties similar to those of other proteins known to contain these domains.

The domain and protein similarity information for the invention suggests that this gene may function as “5-Hydroxytryptamine receptor”. As such, the NOV8 protein of the invention may function in Seizures, Alzheimer disease, sleep, appetite, thermoregulation, pain perception, hormone secretion, and sexual behavior, mental depression, migraine, epilepsy, obsessive-compulsive Behavior (schizophrenia), and affective disorder.

The nucleic acids and proteins of the invention are useful in potential therapeutic applications implicated in various pathologies/disorders described. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

NOV8 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 20 to 50. In another embodiment, a NOV8 epitope is from about amino acids 120 to 140. In additional embodiments, a NOV8 epitope is from about amino acids 160 to 180, from about amino acids 200 to 240, from about amino acids 245 to 280, from about 290 to 325, and from about amino acids 350 to 375. These novel proteins can be used in assay systems for functional analysis of various human disorders, which are useful in understanding of pathology of the disease and development of new drug targets for various disorders.

NOV9

A disclosed NOV9 nucleic acid (also referred to as AC013554_da1) of 620 nucleotides (SEQ ID NO:31) encoding a novel Thioredoxin-like protein is shown in Table 9A. An open reading frame was identified beginning with an ATG initiation codon at nucleotides 282-284 and ending with a TGA codon at nucleotides 618-620. Untranslated regions upstream from the initiation codon and downstream from the termination codon are underlined in Table 9A. The start and stop codons are in bold letters.

TABLE 9A
NOV9 Nucleotide Sequence
(SEQ ID NO:31)

TGTAAAACAAGACCAGGCACAAGAAGGGTGACATCCCCAAGTCCCCAGAAGAAGCCATCCAGCACAAGGAGGGTG
ACATTCCCAAGTCTCCAAAACAAGCCATCCAGCCCAAGGAGGGTGACATTCCCAAGTCCCTAGAGGAAGCCATCC
CACCCAAGGAGATTGACATCCCCAAGTCCCCA2AAGAAACCATCCACCCCAAGGAGGATGACAGCCCCAAGTCCC
TAGAAGAAGCCACCCCATCCAAGGAGGGTGACATCCTAAAGCCTGAAGAAGAAACAATGGAGTTCCCGGAGGGGG
ACAAGGTGAAAGTGATCCTGAGCAAGGAGGACTTTGAGACATCACTGAAGGAGGCCGGGGAGAGGCTGGTGGCTG
TGGACTTCTCGGCCACGTGGTGTGGGCCCTGCAGGACCATCAGACCATTCTTCCATGCCCTGTCTGTGAAGCATG
AGGATGTGGTGTTCCTGGAGGTGGACGCTGACAACTGTGAGGAGGTCGTGAGAGAGTGCGCCATCATGTGTGTCC
CAACCTTTCAGTTTTATAAAAAAGAAGAAAAGGTGGATGAACTTTGCCGCGCCCTTAAGGAAAAACTTGAAGCAG
TCATTGCAGAATTAAAGTAA

Variant sequences of NOV9 are included in Example 2, Table 55. A variant sequence can include a single nucleotide polymorphism (SNP). A SNP can, in some instances, be referred to as a “cSNP” to denote that the nucleotide sequence containing the SNP originates as a cDNA.

In a search of public sequence databases, the NOV9 nucleic acid sequence, located on the p31 region of chromosome 2 has 812 of 1089 bases 74%) identical to a 5-HT5B serotonin receptor mRNA from Mus musculus (GENBANK-ID: X69867) (E=1.8e⁻¹¹⁵). Public nucleotide databases include all GenBank databases and the GeneSeq patent database.

The NOV9 protein (SEQ ID NO:32) encoded by SEQ ID NO:31 is 112 amino acid residues in length, has a molecular weight of 12746.6 Daltons, and is presented using the one-letter amino acid code in Table 9B. The Psort profile for NOV9 predicts that this sequence has a signal sequence and is likely to be localized in the cytoplasm with a certainty of 0.6500.

TABLE 9B
Encoded NOV9 protein sequence

(SEQ ID NO:32)

MEFPEGDKVKVILSKEDFETSLKEAGERLVAVDFSATWCGPCRTIRPFFHLSVKHEDVVFLEVDADNCEEVV
RECAIMCVPTFQFYKKEEKVDELCGALKEKLEAVIAELK

In a BLAST search of public sequence databases, it was found, for example, that the full amino acid sequence of NOV9 was found to have 65 of 103 amino acid residues (63%) identical to, and 80 of 103 amino acid residues (77%) similar to, the 105 amino acid residue THIOREDOXIN - Equus caballus (ACC: O97508) (E=3.2e⁻³²) and 63 of 103 amino acid residues (61%) identical to, and 80 of 103 amino acid residues (77%) similar to, the 104 amino acid residue THIOREDOXIN (ATL-DERIVED FACTOR) (ADF) (SURFACE ASSOCIATED SULPHYDRYL PROTEIN) (SASP) - Homo sapiens (ACC: P10599) (E=2.2e⁻³¹).

NOV9 has homology to the proteins shown in the BLASTP data in Table 9C.

TABLE 9C
BLAST results for NOV9

| Gene Index/ | | Length | Identity | Positives | |
|---|---|---|---|---|---|
| Identifier | Protein/ Organism | (aa) | (%) | (%) | Expect |
| 097508 | Thioredoxin | 105 | 65/103 | 80/103, | 1 e − 33 |
| | [Equus caballus] | | (63%) | (78%) | |
| THIO₋ | Thioredoxin | 104 | 65/103 | 80/103, | 2 e − 33 |
| SHEEP | [Ovis aries] | | (63%) | (78%) | |
| THIO₋ | Thioredoxin | 104 | 65/103 | 80/103, | 3 e − 33 |
| BOVIN | [Bos taurus] | | (63%) | (78%) | |
| THIO₋ | Thioredoxin | 104 | 64/103 | 81/103, | 4 e − 33 |
| MACMU | [Macaca mulatta] | | (62%) | (79%) | |
| THIO₋RAT | Thioredoxin | 104 | 63/102 | 80/102, | 5 e − 33 |
| | [Rattus | | (62%) | (78%) | |
| | norvegicus] | | | | |

A multiple sequence alignment is given in Table 9D, with the NOV9 protein being shown on line 1 in Table 9D in a ClustalW analysis, and comparing the NOV9 protein with the related protein sequences shown in Table 9C. This BLASTP data is displayed graphically in the ClustalW in Table 9D.

TABLE 9D
ClustalW Analysis of NOV9

1. >NOV9; SEQ ID NO:32
2. >O97508/Thioredoxin [Equus caballus]; SEQ ID NO:61
3. >THIO_SHEEP/Thioredoxin [Ovis aries]; SEQ ID NO:62
4. >THIO_BOVIN/Thioredoxin [Bos taurus]; SEQ ID NO:63
5. >THIO_MACMU/Thioredoxin [Macaca mulatta]; SEQ ID NO:64
6. >THIO_RAT/Thioredoxin [Rattus norvegicus]; SEQ ID NO:65

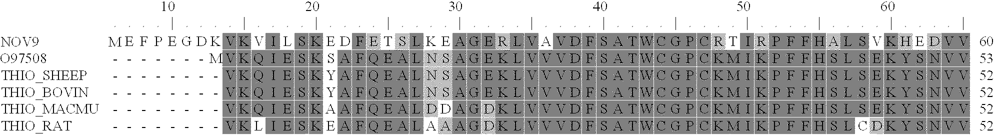

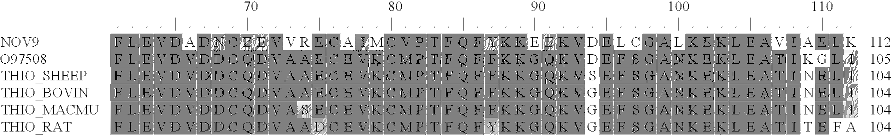

The presence of identifiable domains in the protein disclosed herein was determined by searches using algorithms such as Pfam. Table 9E lists the domain description from DOMAIN analysis results against NOV9.

TABLE 9E
Domain Analysis of NOV9

| | | Score | |
|---|---|---|---|
| Model | Range | (bits) | E value |
| Thioredoxin | 7-110 | 89.4 | 1e-19 |

The presence of protein regions in NOV9 that are homologous to a leucine-rich repeat domain is consistent with the identification of NOV9 protein as a Thioredoxin-like protein. This indicates that the NOV9 sequence has properties similar to those of other proteins known to contain these domains.

The domain and protein similarity information for the invention suggests that this gene may function as “Thioredoxin”. As such, the NOV9 protein of the invention may function in Inflamation, Autoimmune disorders, Aging and Cancer or other thioredoxin related disorders.

The nucleic acids and proteins of the invention are useful in potential therapeutic applications implicated in various pathologies/disorders described. Potential therapeutic uses for the invention includes, for example; protein therapeutic, small molecule drug target, antibody target (Therapeutic, Diagnostic, Drug targeting/Cytotoxic antibody), diagnostic and/or prognostic marker, gene therapy (gene delivery/gene ablation), research tools, tissue regeneration in vitro and in vivo (regeneration for all these tissues and cell types composing these tissues and cell types derived from these tissues).

NOV9 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. These novel proteins can be used in assay systems for functional analysis of various human disorders, which are useful in understanding of pathology of the disease and development of new drug targets for various disorders.

TABLE 10
Sequences and Corresponding SEQ ID Numbers

| | | SEQ ID NO | SEQ ID NO | |
|---|---|---|---|---|
| NOVX | Internal Identification | (nucleic acid) | (polypeptide) | Homology |
| 1a | sggc_draft_dj881p19_20 | 1 | 2 | Wnt-like |
| | 000725; | | | |
| | sggc_draft_dj881p19_20 | | | |
| | 000725-a; | | | |
| | X56842_da1_CG55702-01 | | | |
| 1b | GM_AL136379_A | 3 | 4 | Wnt-like |
| 1c | CG55702-04 | 5 | 6 | Wnt-like |
| 2a | 30370359_da1 | 7 | 8 | Zinc transporter- |
| | | | | like |
| 2b | CG57799-01 | 9 | 10 | Zinc transporter- |
| | | | | like |
| 2c | CG57799-02 | 11 | 12 | Zinc transporter- |
| | | | | like |
| 3a | SC126413398 | 13 | 14 | Mitsugumin29-like |
| 3b | CG55861-02 | 15 | 16 | Mitsugumin29-like |
| 4a | 20760813.0.1. | 17 | 18 | Slit-3-like |
| 4b | CG51514-05 | 19 | 20 | Slit-3-like |
| 5a | 133783508ext | 21 | 22 | LRR/GPCR-like |
| 5b | BE304119ext | 23 | 24 | LRR/GPCR-like |
| 6 | jgigc_draft_citb- | 25 | 26 | Major |
| | e1_2540b15_20000803 | | | histocompatability |
| | | | | complex enhancer |
| | | | | protein MAD3-like |
| 7 | GMAP001948_A | 27 | 28 | Interleukin 9-like |
| 8 | SC129285515_A | 29 | 30 | 5- |
| | | | | Hydroxytryptamine |
| | | | | receptor-like |
| 9 | AC013554_da1 | 31 | 32 | Thioredoxin-like |

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, 31 or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, or 31 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g. using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would farther comprise at least 6 contiguous nucleotides SEQ ID NOS:1, 3, 5, 7, 9, 11, 13, 15, 17, 19, 21, 23, 25, 27, 29, and 31, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 29,and31, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, 27, 29 or 31 is one that is sufficiently complementary to the nucleotide sequence shown SEQ ID NOS:1, 3, 5, 7, 9, 11, 13, 15, 17, 19, 21, 23, 25, 27, 29 or 31 that it can hydrogen bond with little or no mismatches to the nucleotide sequence shown SEQ ID NOS:1, 3, 5, 7, 9, 11, 13, 15, 17, 19, 21, 23, 25, 27, 29, and 31, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, and 31, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29 or 31; or an anti-sense strand nucleotide sequence of SEQ ID NOS:1, 3, 5, 7, 9, 11, 13, 15, 17, 19, 21, 23, 25, 27, 29, 31; or of a naturally occurring mutant of SEQ ID NOS:1, 3, 5, 7, 9, 11, 13, 15, 17, 19, 21, 23, 25, 27, 29,and31.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, 27, 29, or 31 that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, and 31 due to degeneracy of the genetic code and thus encode the same NOVX proteins as that encoded by the nucleotide sequences shown in SEQ ID NOS:1, 3, 5, 7, 9, 11, 13, 15, 17, 19 21, 23, 25, 27, 29, and 31. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, and 32.

In addition to the human NOVX nucleotide sequences shown in SEQ ID NOS:1, 3, 5, 7, 9, 11, 13, 15, 17, 19, 21, 23, 25, 27, 29, and 31, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, 27, 29, and 31 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 21, 23, 25, 27, 29, and 31. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (ie., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, and 31, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, and 31, or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, 27, 29, and 31, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, and 31, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, and 32. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:2, 4, 6, 8, 10, 12, 14, 16, 18, 20, 22, 24, 26, 28, 30 or 32 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30 or 32. Preferably, the protein encoded by the nucleic acid molecule is at least about 60% homologous to SEQ ID NOS:2, 4, 6, 8, 10, 12, 14, 16, 18, 20, 22, 24, 26, 28, 30 or 32; more preferably at least about 70% homologous SEQ ID NOS:2, 4, 6, 8, 10, 12, 14, 16, 18, 20, 22, 24, 26, 28, 30, and 32; still more preferably at least about 80% homologous to SEQ ID NOS:2, 4, 6, 8, 10, 12, 14, 16, 18, 20, 22, 24, 26, 28, 30, and 32; even more preferably at least about 90% homologous to SEQ ID NOS:2, 4, 6, 8, 10, 12, 14, 16, 18, 20, 22, 24, 26, 28, 30, and 32; and most preferably at least about 95% homologous to SEQ ID NOS:2, 4, 6, 8, 10, 12, 14, 16, 18, 20, 22, 24, 26, 28, 30, and 32.

An isolated nucleic acid molecule encoding an NOVX protein homologous to the protein of SEQ ID NOS:2, 4, 6, 8, 10, 12, 14, 16, 18, 20, 22, 24, 26, 28, 30, and 32 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, and 31, such that one or more amino acid substitutions, additions or deletions are introduced into the encoded protein.

Mutations can be introduced into SEQ ID NOS:2, 4, 6, 8, 10, 12, 14, 16, 18, 20, 22, 24, 26, 28, 30, and 32 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17, 19, 21, 23, 25, 27, 29, and 31, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, BFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, and 31, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30 and 32, or antisense nucleic acids complementary to an NOVX nucleic acid sequence of SEQ ID NOS:1, 3, 5, 7, 9, 11, 13, 15, 17, 19, 21, 23, 25, 27, 29, and 31, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonulceotide can be, for example, about 5, 10, 15, 20, 25, 30, 35, 40, 45 or 50 nucleotides in length. An antisense nucleic acid of the invention can be constructed using chemical synthesis or enzynatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subdloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, 27, 29, and 31). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann.

N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, and 32. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, and 32 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, and 32) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, and 32. In other embodiments, the NOVX protein is substantially homologous to SEQ ID NOS:2, 4, 6, 8, 10, 12, 14, 16, 18, 20, 22, 24, 26, 28, 30, and 32, and retains the functional activity of the protein of SEQ ID NOS:2, 4, 6, 8, 10, 12, 14, 16, 18, 20, 22, 24, 26, 28, 30, and 32, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, and 32, and retains the functional activity of the NOVX proteins of SEQ ID NOS:2, 4, 6, 8, 10, 12, 14, 16, 18, 20, 22, 24, 26, 28, 30, and 32.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, and 31.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, and 32), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g. mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline pbosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

The invention encompasses antibodies and antibody fragments, such as F_{ab} or (F_{ab})₂, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown SEQ ID NOS:2, 4, 6, 8, 10, 12, 14, 16, 18, 20, 22, 24, 26, 28, 30, and 32 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, NOVX protein sequences of SEQ ID NOS:2, 4, 6, 8, 10, 12, 14, 16, 18, 20, 22, 24, 26, 28, 30, 32, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F(_{ab})₂ fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS:2, 4, 6, 8, 10, 12, 14, 16, 18, 20, 22, 24, 26, 28, 30, 32, or a derivative, fragment, analog or homolog thereof. Some of these proteins are discussed below.

For the production of polyclonal antibodies, various suitable host animals (e.g. rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al, 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) F_{V} fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No.171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et a., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 29, and 31 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, and 31), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, and 31 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g. by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g. a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g., developmental disorders, endocrine disorders, vascular disorders, infectious disease, anorexia, cancer, neurodegenerative disorders, lung disorders, reproductive disorders, Alzheimer's Disease, Parkinson's Disease, immune disorders, and hematopoietic disorders, or other disorders related to cell signal processing and metabolic pathway modulation, and various cancers, and infectious disease(possesses anti-microbial activity). In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, ef al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, 27, 29, and 31, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, and 31 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include developmental disorders, endocrine disorders, vascular disorders, infectious disease, anorexia, cancer, neurodegenerative disorders, lung disorders, reproductive disorders, Alzheimer's Disease, Parkinson's Disease, immune disorders, and hematopoietic disorders, or other disorders related to cell signal processing and metabolic pathway modulation, and various cancers, and infectious disease (possesses anti-microbial activity). The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, 27, 29,and31,or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders [the disorders include developmental disorders, endocrine disorders, vascular disorders, infectious disease, anorexia, cancer, neurodegenerative disorders, lung disorders, reproductive disorders, Alzheimer's Disease, Parkinson's Disease, immune disorders, and hematopoietic disorders, or other disorders related to cell signal processing and metabolic pathway modulation, and various cancers, and infectious disease (possesses anti-microbial activity)]. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include endocrine disorders; developmental disorders; gastrointestinal diseases; lung diseases; respiratory disorders; vascular diseases; blood disorders; autoimmune and immune disorders; multiple sclerosis; inflammatory disorders and Hepatitis C; Trauma; regeneration (in vitro and in vivo); viral/bacterial/parasitic infections; hyperthyroidism; hypothyroidism; endometriosis; fertility; angiogenesis; hypertension; stroke; ischemia; arteriosclerosis; aneurysms; stroke; and bleeding disorders; Bare lymphocytic syndrome; type II; hereditary spherocytosis; elliptocytosis; pyropoikilocytosis; hemolytic anemia; Werner syndrome (scleroderma-like skin changes); juvenile rheumatoid arthritis; Graves disease; wound healing; X-linked mental retardation; and fertility disorders; psychotic and neurological disorders; neuronal degeneration; including but not limited to Parkinson's and Alzheimer's Disease; dysplastic nevi and cancer; including but not limited to; glioma; leukemia; melanoma; pancreatic adenocarcinoma; non-Hodgkin's lymphoma; renal cancer; hepatocellular carcinomas; and myeloid leukemia lung or breast cancer, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: developmental disorders, endocrine disorders, vascular disorders, infectious disease, anorexia, cancer, neurodegenerative disorders, lung disorders, reproductive disorders, Alzheimer's Disease, Parkinson's Disease, immune and autoimmune disorders, and hematopoietic disorders, or other disorders related to cell signal processing and metabolic pathway modulation.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: developmental disorders, endocrine disorders, vascular disorders, infectious disease, anorexia, cancer, neurodegenerative disorders, lung disorders, reproductive disorders, Alzheimer's Disease, Parkinson's Disease, immune and autoimmune disorders, and hematopoietic disorders, or other disorders related to cell signal processing and metabolic pathway modulation.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

### Example 1.

Quantitative expression analysis of clones in various cells and tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources), Panel 4 (containing cells and cell lines from normal cells and cells related to inflammatory conditions) and Panel CNSD.01 (containing samples from normal and diseased brains).

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgC12, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28 S and 18 S ribosomal RNA staining intensity ratio as a guide (2:1 to 2.5:1 28 s:18 s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28 S and 18 S ribosomal RNA staining intensity ratio as a guide (2:1 to 2.5:1 28 s:18 s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene ,La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-1 3 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1- 7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28 S and 18 S ribosomal RNA staining intensity ratio as a guide (2:1 to 2.5:1 28 s: 18 s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

NOV1 (NOV1a-c)

Expression of gene NOV1a (and its variants) was assessed using the primer-probe set Ag2445, described in Table 11. Results from RTQ-PCR runs are shown in Tables 12 and 13.

TABLE 11
Probe Name Ag2445

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-GCCCCACTCGGATACTTCT-3′ | 59.1 | 19 | 34 | 66 |
| Probe | FAM-5′- | 71.3 | 26 | 53 | 67 |
| | TACTCCTCTGCAGCCTGAAGCAGGCT-3′- | | | | |
| | TAMRA | | | | |
| Reverse | 5′-GGAATACTGTGGCCCAACA-3′ | 59.4 | 19 | 111 | 68 |

TABLE 12
Panel 1.3D

| | Relative Expression (%) | |
|---|---|---|
| | 1.3dtm4280f_ | 1.3dtm4393f_ |
| Tissue Name | ag2445 | ag2445 |
| Liver adenocarcinoma | 0.0 | 0.0 |
| Pancreas | 0.0 | 0.0 |
| Pancreatic ca. CAPAN 2 | 0.0 | 0.0 |
| Adrenal gland | 0.0 | 0.0 |
| Thyroid | 0.0 | 0.0 |
| Salivary gland | 11.4 | 10.3 |
| Pituitary gland | 0.0 | 0.0 |
| Brain (fetal) | 0.0 | 0.0 |
| Brain (whole) | 0.0 | 0.0 |
| Brain (amygdala) | 0.0 | 0.0 |
| Brain (cerebellum) | 0.0 | 0.0 |
| Brain (hippocampus) | 0.0 | 0.0 |
| Brain (substantia nigra) | 0.0 | 0.0 |
| Brain (thalamus) | 0.0 | 0.0 |
| Cerebral Cortex | 0.0 | 0.0 |
| Spinal cord | 0.0 | 0.0 |
| CNS ca. (glio/astro) U87-MG | 0.0 | 0.0 |
| CNS ca. (glio/astro) U-118-MG | 0.0 | 0.0 |
| CNS ca. (astro) SW1783 | 0.0 | 0.0 |
| CNS ca.* (neuro; met) SK-N-AS | 0.0 | 0.0 |
| CNS ca. (astro) SF-539 | 0.0 | 0.0 |
| CNS ca. (astro) SNB-75 | 3.6 | 0.0 |
| CNS ca. (glio) SNB-19 | 0.0 | 0.0 |
| CNS ca. (glio) U251 | 0.0 | 0.0 |
| CNS ca. (glio) SF-295 | 0.0 | 0.0 |
| Heart (fetal) | 0.0 | 0.0 |
| Heart | 0.0 | 0.0 |
| Fetal Skeletal | 0.0 | 0.0 |
| Skeletal muscle | 0.0 | 0.0 |
| Bone marrow | 0.0 | 0.0 |
| Thymus | 0.0 | 0.0 |
| Spleen | 0.0 | 0.0 |
| Lymph node | 0.0 | 0.0 |
| Colorectal | 0.0 | 0.0 |
| Stomach | 0.0 | 0.0 |
| Small intestine | 0.0 | 0.0 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 10.8 |
| Colon ca. CaCo-2 | 0.0 | 0.0 |
| 83219 CC Well to Mod Diff | 0.0 | 0.0 |
| (ODO3866) | | |
| Colon ca. HCC-2998 | 0.0 | 0.0 |
| Gastric ca.* (liver met) NCI-N87 | 0.0 | 0.0 |
| Bladder | 0.0 | 0.0 |
| Trachea | 62.8 | 66.0 |
| Kidney | 0.0 | 0.0 |
| Kidney (fetal) | 0.0 | 0.0 |
| Renal ca. 786-0 | 0.0 | 0.0 |
| Renal ca. A498 | 0.0 | 7.6 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 59.5 | 100.0 |
| Lung (fetal) | 3.7 | 0.0 |
| Lung ca. (small cell) LX-1 | 0.0 | 0.0 |
| Lung ca. (small cell) NCI-H69 | 0.0 | 0.0 |
| Lung ca. (s. cell var.) SHP-77 | 0.0 | 0.0 |
| Lung ca. (large cell) NCI-H460 | 0.0 | 0.0 |
| Lung ca. (non-sm. cell) A549 | 1.4 | 0.0 |
| Lung ca. (non-s. cell) NCI-H23 | 0.0 | 0.0 |
| Lung ca (non-s. cell) HOP-62 | 0.0 | 0.0 |
| Lung ca. (non-s. cl) NCI-H522 | 0.0 | 0.0 |
| Lung ca. (squam.) SW 900 | 3.0 | 0.0 |
| Lung ca. (squam.) NCI-H596 | 0.0 | 0.0 |
| Mammary gland | 2.7 | 0.0 |
| Breast ca.* (pl. effusion) MCF-7 | 7.5 | 5.8 |
| Breast ca.* (pl. ef) MDA-MB-231 | 0.0 | 0.0 |
| Breast ca.* (pl. effusion) T47D | 0.0 | 0.0 |
| Breast ca. BT-549 | 0.0 | 0.0 |
| Breast ca. MDA-N | 0.0 | 0.0 |
| Ovary | 0.0 | 0.0 |
| Ovarian ca. OVCAR-3 | 0.0 | 0.0 |
| Ovarian ca. OVCAR-4 | 0.0 | 6.5 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca.* (ascites) SK-OV-3 | 6.1 | 12.3 |
| Uterus | 0.0 | 0.0 |
| Placenta | 100.0 | 82.4 |
| Prostate | 7.2 | 10.3 |
| Prostate ca.* (bone met) PC-3 | 0.0 | 0.0 |
| Testis | 0.0 | 0.0 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 0.0 | 0.0 |

TABLE 13
Panel 2D

| | Relative Expression (%) | | |
|---|---|---|---|
| | 2dtm4281f_{—} | 2dtm4394f_ | 2dtm4590f_ |
| Tissue Name | ag2445 | ag2445 | ag2445 |
| Normal Colon GENPAK 061003 | 1.6 | 1.8 | 3.3 |
| 83219 CC Well to Mod Diff (ODO3866) | 0.0 | 0.0 | 0.0 |
| 83220 CC NAT (ODO3866) | 0.0 | 0.0 | 0.0 |
| 83221 CC Gr. 2 rectosigmoid (ODO3868) | 0.0 | 0.0 | 0.0 |
| 83222 CC NAT (ODO3868) | 0.0 | 0.0 | 0.0 |
| 83235 CC Mod Diff (ODO3920) | 0.0 | 0.0 | 0.0 |
| 83236 CC NAT (ODO3920) | 0.0 | 0.0 | 0.0 |
| 83237 CC Gr. 2 ascend colon (ODO3921) | 0.0 | 0.0 | 0.0 |
| 83238 CC NAT (ODO3921) | 0.0 | 0.0 | 0.0 |
| 83241 CC from Partial Hepatectomy | | | |
| (ODO4309) | 0.0 | 0.0 | 0.0 |
| 83242 Liver NAT (ODO4309) | 0.0 | 0.0 | 0.0 |
| 87472 Colon mets to lung (OD04451-01) | 13.4 | 4.4 | 8.0 |
| 87473 Lung NAT (OD04451-02) | 50.7 | 38.4 | 49.3 |
| Normal Prostate Clontech A+ 6546-1 | 13.7 | 6.6 | 76.3 |
| 84140 Prostate Cancer (OD04410) | 2.9 | 5.9 | 4.7 |
| 84141 Prostate NAT (OD04410) | 6.3 | 6.2 | 16.4 |
| 87073 Prostate Cancer (OD04720-01) | 9.8 | 28.1 | 22.1 |
| 87074 Prostate NAT (OD04720-02) | 29.7 | 44.4 | 22.4 |
| Normal Lung GENPAK 061010 | 46.7 | 50.3 | 66.9 |
| 83239 Lung Met to Muscle (ODO4286) | 0.0 | 0.0 | 0.0 |
| 83240 Muscle NAT (ODO4286) | 0.0 | 0.0 | 0.0 |
| 84136 Lung Malignant Cancer (OD03126) | 3.0 | 7.6 | 5.7 |
| 84137 Lung NAT (OD03126) | 31.6 | 54.0 | 56.6 |
| 84871 Lung Cancer (OD04404) | 11.2 | 10.6 | 7.0 |
| 84872 Lung NAT (OD04404) | 54.0 | 25.3 | 37.4 |
| 84875 Lung Cancer (OD04565) | 16.4 | 5.2 | 3.2 |
| 84876 Lung NAT (OD04565) | 15.8 | 35.8 | 24.1 |
| 85950 Lung Cancer (OD04237-01) | 0.0 | 0.0 | 2.0 |
| 85970 Lung NAT (OD04237-02) | 71.2 | 74.2 | 100.0 |
| 83255 Ocular Mel Met to Liver (ODO4310) | 0.0 | 0.0 | 0.0 |
| 83256 Liver NAT (ODO4310) | 0.0 | 0.0 | 0.0 |
| 84139 Melanoma Mets to Lung (OD04321) | 0.0 | 0.0 | 0.0 |
| 84138 Lung NAT (OD04321) | 100.0 | 100.0 | 77.4 |
| Normal Kidney GENPAK 061008 | 0.0 | 1.2 | 0.0 |
| 83786 Kidney Ca. Nuclear grade 2 (OD04338) | 0.0 | 0.0 | 0.0 |
| 83787 Kidney NAT (OD04338) | 0.0 | 0.0 | 0.0 |
| 83788 Kidney Ca Nuclear grade 1/2 (OD04339) | 0.0 | 0.0 | 0.0 |
| 83789 Kidney NAT (OD04339) | 3.2 | 0.0 | 0.0 |
| 83790 Kidney Ca. Clear cell type (OD04340) | 0.0 | 0.0 | 0.0 |
| 83791 Kidney NAT (OD04340) | 0.0 | 0.0 | 0.0 |
| 83792 Kidney Ca. Nuclear grade 3 (OD04348) | 0.0 | 0.0 | 0.0 |
| 83793 Kidney NAT (OD04348) | 0.0 | 0.0 | 0.0 |
| 87474 Kidney Cancer (OD04622-01) | 0.0 | 0.0 | 4.3 |
| 87475 Kidney NAT (OD04622-03) | 0.0 | 0.0 | 0.0 |
| 85973 Kidney Cancer (OD04450-01) | 0.0 | 0.0 | 0.0 |
| 85974 Kidney NAT (OD04450-03) | 0.0 | 0.0 | 4.9 |
| Kidney Cancer Clontech 8120607 | 0.0 | 0.0 | 0.0 |
| Kidney NAT Clontech 8120608 | 0.0 | 0.0 | 0.0 |
| Kidney Cancer Clontech 8120613 | 7.0 | 2.4 | 2.2 |
| Kidney NAT Clontech 8120614 | 0.0 | 0.0 | 0.0 |
| Kidney Cancer Clontech 9010320 | 0.0 | 0.0 | 0.6 |
| Kidney NAT Clontech 9010321 | 0.0 | 0.0 | 0.0 |
| Normal Uterus GENPAK 061018 | 0.0 | 0.0 | 0.0 |
| Uterus Cancer GENPAK 064011 | 0.0 | 0.0 | 0.0 |
| Normal Thyroid Clontech A+ 6570-1 | 0.0 | 0.0 | 0.0 |
| Thyroid Cancer GENPAK 064010 | 0.0 | 0.0 | 0.0 |
| Thyroid Cancer INVITROGEN A302152 | 0.0 | 0.0 | 0.0 |
| Thyroid NAT INVITROGEN A302153 | 0.0 | 0.0 | 0.0 |
| Normal Breast GENPAK 061019 | 0.0 | 1.7 | 0.0 |
| 84877 Breast Cancer (OD04566) | 0.0 | 0.0 | 0.0 |
| 85975 Breast Cancer (OD04590-01) | 0.0 | 0.0 | 0.0 |
| 85976 Breast Cancer Mets (OD04590-03) | 0.0 | 0.0 | 0.0 |
| 87070 Breast Cancer Metastasis (OD04655-05) | 0.0 | 0.0 | 0.0 |
| GENPAK Breast Cancer 064006 | 0.0 | 1.7 | 0.0 |
| Breast Cancer Res. Gen. 1024 | 1.3 | 0.0 | 2.1 |
| Breast Cancer Clontech 9100266 | 0.0 | 0.0 | 1.9 |
| Breast NAT Clontech 9100265 | 1.5 | 0.0 | 4.8 |
| Breast Cancer INVITROGEN A209073 | 0.0 | 0.0 | 0.0 |
| Breast NAT INVITROGEN A2090734 | 0.0 | 0.0 | 0.0 |
| Normal Liver GENPAK 061009 | 0.0 | 0.0 | 0.0 |
| Liver Cancer GENPAK 064003 | 0.0 | 0.0 | 0.0 |
| Liver Cancer Research Genetics RNA 1025 | 0.0 | 0.0 | 0.0 |
| Liver Cancer Research Genetics RNA 1026 | 24.7 | 54.3 | 63.7 |
| Paired Liver Cancer Tissue Research Genetics | 0.0 | 0.0 | 0.0 |
| RNA 6004-T | | | |
| Paired Liver Tissue Research Genetics RNA | 0.0 | 0.0 | 0.0 |
| 6004-N | | | |
| Paired Liver Cancer Tissue Research Genetics | 54.3 | 49.0 | 98.6 |
| RNA 6005-T | | | |
| Paired Liver Tissue Research Genetics RNA | 0.0 | 0.0 | 0.0 |
| 6005-N | | | |
| Normal Bladder GENPAK 061001 | 0.0 | 0.0 | 0.0 |
| Bladder Cancer Research Genetics RNA 1023 | 0.0 | 0.0 | 0.0 |
| Bladder Cancer INVITROGEN A302173 | 0.0 | 0.0 | 0.0 |
| 87071 Bladder Cancer (OD04718-01) | 2.1 | 1.9 | 0.7 |
| 87072 Bladder Normal Adjacent (OD04718-03) | 0.0 | 0.0 | 0.0 |
| Normal Ovary Res. Gen. | 0.0 | 0.0 | 0.0 |
| Ovarian Cancer GENPAK 064008 | 8.4 | 1.6 | 5.8 |
| 87492 Ovary Cancer (OD04768-07) | 0.0 | 0.0 | 0.0 |
| 87493 Ovary NAT (OD04768-08) | 0.0 | 0.0 | 0.0 |
| Normal Stomach GENPAK 061017 | 0.0 | 0.0 | 0.0 |
| Gastric Cancer Clontech 9060358 | 0.0 | 0.0 | 0.0 |
| NAT Stomach Clontech 9060359 | 0.0 | 0.0 | 0.0 |
| Gastric Cancer Clontech 9060395 | 3.3 | 0.0 | 0.0 |
| NAT Stomach Clontech 9060394 | 0.0 | 0.0 | 0.0 |
| Gastric Cancer Clontech 9060397 | 0.0 | 0.0 | 0.0 |
| NAT Stomach Clontech 9060396 | 0.0 | 0.0 | 0.0 |
| Gastric Cancer GENPAK 064005 | 0.0 | 0.0 | 0.0 |

Panel 1.3D Summary:

Ag2445 Results from two replicate experiments using the same probe/primer set are in good agreement with minor differences in expression levels but not tissue distribution. Significant expression of the NOV1a gene is limited to lung, trachea, and placenta. Therefore, NOV1a nucleic acids can be used as a marker for these tissues.

Panel 2D Summary:

Ag2445 Results from three replicate experiments using the same probe/primer set are in moderate agreement. Expression of the NOV1a gene is highest in normal lung tissue (CT=31.2). This observation is consistent with what was seen in Panel 1.3D. In addition, there is significant but low expression of this gene in samples derived from liver cancer and normal prostate tissue. Of note is the consistent dysregulation in NOV1a gene expression between normal lung and lung cancer samples, in which 5 of 5 samples show prominent expression in normal matched lung tissue when compared to cancerous tissue. Thus, the expression of this gene could be used to distinguish normal lung tissue from diseased (cancer) lung tissue. In addition, therapeutic modulation of the activity of the NOV1a gene product is of utility in the treatment of lung cancer.

Panel 4D Summary:

Ag2445 Expression of the NOV1a gene is low/undetectable (CT values>35) across the samples on this panel (data not shown).

Panel CNSD.01 Summary:

Ag2445 Expression of the NOV1a gene is low/undetectable (CT values>35) across the samples on this panel (data not shown).

NOV2 (NOV2a-c)

Expression of gene NOV2a and the variants were assessed using the primer-probe sets Ag3334 and Ag4403, described in Tables 14 and 15. Ag4403 contains a single base insertion in 5′end of rev primer relative to the NOV2a and NOV2C sequences and is not expected to alter RTQ-PCR results. Results from RTQ-PCR runs are shown in Table 16.

TABLE 14
Probe Name Ag3334

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-CGTCATGGAGTTTCTTGAAAGA-3′ | 59.3 | 22 | 288 | 69 |
| Probe | FAM-5′- | 67 | 26 | 329 | 70 |
| | AAGCTGCCAAGATGTATGCTTTCACA- | | | | |
| | 3′-TAMRA | | | | |
| Reverse | 5′-TCTGTTGGAGTTCCACACTTTC-3′ | 59.2 | 22 | 358 | 71 |

TABLE 15
Probe Name Ag44O3

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-ACTCACTCACCATTCAGATGGA-3′ | 59.6 | 22 | 1343 | 72 |
| Probe | FAM-5′- | 71.7 | 26 | 1365 | 73 |
| | ATCTCCAGTTGACCAGGACCCCGACT- | | | | |
| | 3′-TAMRA | | | | |
| Reverse | 5′-CTAGTTCACAGGGGTCTTCACA-3′ | 59.3 | 22 | 1399 | 74 |

TABLE 16
Panel 4.1D

| | Relative | | Relative |
|---|---|---|---|
| | Expression (%) | | Expression (%) |
| | 4.1dx4tm6648f | | 4.1dx4tm6648f |
| Tissue Name | _ag4403_a2 | Tissue Name | _ag4403_a2 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.5 | 93779_HUVEC | 0.0 |
| CD28/anti-CD3 | | (Endothelial)_IFN gamma | |
| 93770_Secondary Tr1_anti- | 1.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 | |
| | | ng/ml) and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml) ** | |
| 93566_primary Th2_resting dy | 0.0 | 93347_Small Airway | 0.0 |
| 4-6 in IL-2 | | Epithelium_none | |
| 93567_primary Tr1_resting dy | 0.3 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 2.7 | 92668_Coronery Artery | 0.0 |
| lymphocyte_anti-CD28/anti- | | SMC_resting | |
| CD3 | | | |
| 93352_CD45RO CD4 | 1.2 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 0.0 |
| CD28/anti-CD3 | | | |
| 93353_chronic CD8 | 0.0 | 93108_astrocytes_TNFa (4 | 0.0 |
| Lymphocytes 2ry_resting dy 4- | | ng/ml) and IL1b (1 ng/ml) | |
| 6 in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 2.4 | 93579_CCD1106 | 0.0 |
| Th1/Th2/Tr1_anti-CD95 CH11 | | (Keratinocytes)_none | |
| 93103_LAK cells_resting | 1.1 | 93580_CCD1106 | 0.0 |
| | | (Keratinocytes)_TNFa and | |
| | | IFNg ** | |
| 93788_LAK cells_IL-2 | 1.0 | 93791_Liver Cirrhosis | 0.0 |
| 93787_LAK cells_IL-2 + IL-12 | 0.0 | 93577_NCI-H292 | 0.0 |
| 93789_LAK cells_IL-2 + IFN | 1.6 | 93358_NCI-H292_IL-4 | 0.0 |
| gamma | | | |
| 93790_LAK cells_IL-2 + IL-18 | 0.0 | 93360_NCI-H292_IL-9 | 0.0 |
| 93104_LAK | 0.0 | 93359_NCI-H292_IL-13 | 1.2 |
| cells_PMA/ionomycin and IL- | | | |
| 18 | | | |
| 93578_NK Cells IL-2_resting | 1.2 | 93357_NCI-H292_IFN gamma | 1.2 |
| 93109_Mixed Lymphocyte | 0.0 | 93777_HPAEC_- | 0.0 |
| Reaction_Two Way MLR | | | |
| 93110_Mixed Lymphocyte | 0.0 | 93778_HPAEC_IL-1 beta/TNA | 0.0 |
| Reaction_Two Way MLR | | alpha | |
| 93111_Mixed Lymphocyte | 0.0 | 93254_Normal Human Lung | 0.4 |
| Reaction_Two Way MLR | | Fibroblast_none | |
| 93112_Mononuclear Cells | 0.3 | 93253_Normal Human Lung | 1.2 |
| (PBMCs)_resting | | Fibroblast_TNFa (4 ng/ml) and | |
| | | IL-1b (1 ng/ml) | |
| 93113_Mononuclear Cells | 0.0 | 93257_Normal Human Lung | 0.0 |
| (PBMCs)_PWM | | Fibroblast_IL-4 | |
| 93114_Mononuclear Cells | 0.0 | 93256_Normal Human Lung | 0.0 |
| (PBMCs)_PHA-L | | Fibroblast_IL-9 | |
| 93249_Ramos (B cell)_none | 0.0 | 93255_Normal Human Lung | 1.2 |
| | | Fibroblast_IL-13 | |
| 93250_Ramos (B | 0.0 | 93258_Normal Human Lung | 1.2 |
| cell)_ionomycin | | Fibroblast_IFN gamma | |
| 93349_B lymphocytes_PWM | 0.0 | 93106_Dermal Fibroblasts | 0.0 |
| | | CCD1070_resting | |
| 93350_B lymphoytes_CD40L | 4.1 | 93361_Dermal Fibroblasts | 0.0 |
| and IL-4 | | CCD1070_TNF alpha 4 ng/ml | |
| 92665_EOL-1 | 0.0 | 93105_Dermal Fibroblasts | 3.2 |
| (Eosinophil)_dbcAMP | | CCD1070_IL-1 beta 1 ng/ml | |
| differentiated | | | |
| 93248_EOL-1 | 0.0 | 93772_dermal fibroblast_IFN | 0.0 |
| (Eosinophil)_dbcAMP/PMAion | | gamma | |
| omycin | | | |
| 93356_Dendritic Cells_none | 0.0 | 93771_dermal fibroblast_IL-4 | 0.0 |
| 93355_Dendritic Cells_LPS | 0.0 | 93892_Dermal fibroblasts_none | 0.0 |
| 100 ng/ml | | | |
| 93775_Dendritic Cells_anti- | 0.0 | 99202_Neutrophils_TNFa + LPS | 0.0 |
| CD40 | | | |
| 93774_Monocytes_resting | 0.0 | 99203_Neutrophils_none | 0.0 |
| 93776_Monocytes_LPS 50 | 1.2 | 735010_Colon_normal | 0.0 |
| ng/ml | | | |
| 93581_Macrophages_resting | 0.0 | 735019_Lung_none | 3.4 |
| 93582_Macrophages_LPS 100 | 0.0 | 64028-1_Thymus_none | 1.3 |
| ng/ml | | | |
| 93098_HUVEC | 0.0 | 64030-1_Kidney_none | 100.0 |
| (Endothelial)_none | | | |
| 93099_HUVEC | 0.0 | | |
| (Endothelial)_starved | | | |

Panel 2.2 Summary:

Ag3334 Expression of the NOV2a gene is low/undetectable (CT values>35) across all the samples on this panel (data not shown).

Panel 4D Summary:

Ag3334 Expression of the NOV2a gene low/undetectable (CT values>35) across all the samples on this panel (data not shown).

Panel 4.1D Summary:

Ag4403 Significant expression of the NOV2a gene is limited to kidney (CT=30.8). Thus, NOV2a nucleic acids can be used as a marker to distinguish kidney from other tissues. The NOV2a gene encodes a putative zinc transporter. Members of this family are integral membrane proteins that are found to increase tolerance to divalent metal ions such as cadmium, zinc, and cobalt. These proteins are thought to be efflux pumps that remove these ions from cells [IPR002524]. Therefore, the protein encoded for by the NOV2a gene may be involved in normal cation homeostasis and may be disregulated in diseases of the kidney, such as lupus.

Panel CNS_neurodegeneration_v1.0 Summary:

Ap3334 Expression of the NOV2a gene low/undetectable (CT values>35) across all the samples on this panel (data not shown).

NOV3

Expression of gene NOV3a (and its variant) was assessed using the primer-probe sets Ag1508, Ag2284, and Ag2454, described in Tables 17, 18, and 19. The variant CG55861-02 is recognized by primer-probe set Ag1508 only. Results from RTQ-PCR runs are shown in Tables 20, 21, 22, 23 and 24.

TABLE 17

Probe Name Ag1508

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-ATTTGGCTATCCCTTCAGGTT-3′ | 59 | 21 | 238 | 75 |
| Probe | FAM-5′- | 69.1 | 25 | 263 | 76 |
| | CGGATCCAATATGAGATGCCCCTCT-3′- | | | | |
| | TAMRA | | | | |
| Reverse | 5′-GTCTTGGAGCTGGACTCTTCAT-3′ | 59.9 | 22 | 291 | 77 |

TABLE 18
Probe Name Ag2284

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-TAGTTATCTACCTGCGCTTCCA-3′ | 59.1 | 22 | 399 | 78 |
| Probe | FAM-5′- | 68.5 | 26 | 426 | 79 |
| | TCTACACAGAGAACAAACGCTTCCCG- | | | | |
| | 3′-TAMRA | | | | |
| Reverse | 5′-GAAGGTGAAGGAGACAGTCACA-3′ | 59.3 | 22 | 466 | 80 |

TABLE 19
Probe Name Ag2454

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-ACATCTCCGTGGTGCTCTTT-3′ | 59.7 | 20 | 626 | 81 |
| Probe | TET-5′- | 68.6 | 26 | 648 | 82 |
| | CTTTATCAACTTCTTCCTGTGGGCCG- | | | | |
| | 3′-TAMRA | | | | |
| Reverse | 5′-GGGGTCTCCTTGAACACAAA-3′ | 59.9 | 20 | 685 | 83 |

TABLE 20
Panel 1.2

| | Relative | | Relative |
|---|---|---|---|
| | Expression (%) | | Expression (%) |
| | 1.2tm2126f_ | | 1.2tm2126f_ |
| Tissue Name | ag1508 | Tissue Name | ag1508 |
| Endothelial cells | 0.0 | Renal ca. 786-0 | 0.0 |
| Heart (fetal) | 0.9 | Renal ca. A498 | 0.0 |
| Pancreas | 0.1 | Renal ca. RXF 393 | 0.0 |
| Pancreatic ca. CAPAN 2 | 0.0 | Renal ca. ACHN | 0.0 |
| Adrenal Gland (new lot*) | 2.7 | Renal ca. UO-31 | 0.0 |
| Thyroid | 0.1 | Renal ca. TK-10 | 0.0 |
| Salivary gland | 0.9 | Liver | 0.3 |
| Pituitary gland | 0.0 | Liver (fetal) | 0.0 |
| Brain (fetal) | 0.0 | Liver ca. (hepatoblast) HepG2 | 0.0 |
| Brain (whole) | 0.0 | Lung | 0.0 |
| Brain (amygdala) | 0.0 | Lung (fetal) | 0.0 |
| Brain (cerebellum) | 0.1 | Lung ca. (small cell) LX-1 | 0.0 |
| Brain (hippocampus) | 0.1 | Lung ca. (small cell) NCI-H69 | 0.0 |
| Brain (thalamus) | 0.0 | Lung ca. (s. cell var.) SHP-77 | 0.0 |
| Cerebral Cortex | 0.3 | Lung ca. (large cell) NCI-H460 | 0.0 |
| Spinal cord | 0.0 | Lung ca. (non-sm. cell) A549 | 0.0 |
| CNS ca. (glio/astro) U87-MG | 0.0 | Lung ca. (non-s. cell) NCI-H23 | 0.0 |
| CNS ca. (glio/astro) U-118-MG | 0.0 | Lung ca (non-s. cell) HOP-62 | 0.0 |
| CNS ca. (astro) SW1783 | 0.0 | Lung ca. (non-s. cl) NCI-H522 | 9.4 |
| CNS ca. * (neuro; met) SK-N- | 0.0 | Lung ca. (squam.) SW 900 | 0.2 |
| AS | | | |
| CNS ca. (astro) SF-539 | 0.0 | Lung ca. (squam.) NCI-H596 | 0.0 |
| CNS ca. (astro) SNB-75 | 0.0 | Mammary gland | 0.0 |
| CNS ca. (glio) SNB-19 | 0.0 | Breast ca.* (pl. effusion) MCF-7 | 0.0 |
| CNS ca. (glio) U251 | 0.0 | Breast ca.* (pl. ef) MDA-MB-231 | 0.0 |
| CNS ca. (glio) SF-295 | 0.0 | Breast ca.* (pl. effusion) T47D | 0.0 |
| Heart | 10.7 | Breast ca. BT-549 | 0.0 |
| Skeletal Muscle (new lot*) | 100.0 | Breast ca. MDA-N | 0.0 |
| Bone marrow | 0.1 | Ovary | 0.5 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 0.0 |
| Colorectal | 0.0 | Ovarian ca. OVCAR-8 | 0.0 |
| Stomach | 0.0 | Ovarian ca. IGROV-1 | 0.0 |
| Small intestine | 0.2 | Ovarian ca.* (ascites) SK-OV-3 | 0.0 |
| Colon ca. SW480 | 0.0 | Uterus | 0.2 |
| Colon ca.* (SW480 met) SW620 | 0.0 | Placenta | 0.0 |
| Colon ca. HT29 | 0.0 | Prostate | 0.4 |
| Colon ca. HCT-116 | 0.0 | Prostate ca.* (bone met) PC-3 | 0.0 |
| Colon ca. CaCo-2 | 0.0 | Testis | 0.2 |
| 83219 CC Well to Mod Diff | 0.0 | Melanoma Hs688(A).T | 0.0 |
| (ODO3866) | | | |
| Colon ca. HCC-2998 | 0.0 | Melanoma* (met) Hs688(B).T | 0.0 |
| Gastric ca.* (liver met) NCI- | 0.0 | Melanoma UACC-62 | 0.0 |
| N87 | | | |
| Bladder | 0.2 | Melanoma M14 | 0.0 |
| Trachea | 0.0 | Melanoma LOX IMVI | 0.0 |
| Kidney | 8.9 | Melanoma* (met) SK-MEL-5 | 0.0 |
| Kidney (fetal) | 0.6 | Adipose | 0.4 |

TABLE 21
Panel 1.3D

| | Relative | Relative |
|---|---|---|
| | Expression (%) | Expression (%) |
| | 1.3dx4tm5814f | 1.3dtm4267t_{—} |
| Tissue Name | _ag2284_b1 | ag2454 |
| Liver adenocarcinoma | 0.2 | 0.2 |
| Pancreas | 0.3 | 0.4 |
| Pancreatic ca. CAPAN 2 | 0.0 | 0.0 |
| Adrenal gland | 0.5 | 1.1 |
| Thyroid | 1.2 | 0.8 |
| Salivary gland | 0.4 | 0.1 |
| Pituitary gland | 0.1 | 0.1 |
| Brain (fetal) | 0.0 | 0.0 |
| Brain (whole) | 0.2 | 0.0 |
| Brain (amygdala) | 0.2 | 0.0 |
| Brain (cerebellum) | 0.0 | 0.0 |
| Brain (hippocampus) | 0.0 | 0.4 |
| Brain (substantia nigra) | 0.0 | 0.0 |
| Brain (thalamus) | 0.0 | 0.0 |
| Cerebral Cortex | 0.2 | 0.0 |
| Spinal cord | 0.0 | 0.0 |
| CNS ca. (glio/astro) U87-MG | 0.0 | 0.0 |
| CNS ca. (glio/astro) U-118-MG | 0.2 | 0.3 |
| CNS ca. (astro) SW1783 | 0.0 | 0.0 |
| CNS ca.* (neuro; met) SK-N-AS | 0.0 | 0.0 |
| CNS ca. (astro) SF-539 | 0.0 | 0.0 |
| CNS ca. (astro) SNB-75 | 0.0 | 0.0 |
| CNS ca. (glio) SNB-19 | 0.0 | 0.0 |
| CNS ca. (glio) U251 | 0.1 | 0.0 |
| CNS ca. (glio) SF-295 | 0.0 | 0.0 |
| Heart (fetal) | 1.8 | 0.2 |
| Heart | 2.3 | 0.8 |
| Fetal Skeletal | 100.0 | 100.0 |
| Skeletal muscle | 88.6 | 6.6 |
| Bone marrow | 0.2 | 0.0 |
| Thymus | 0.0 | 0.0 |
| Spleen | 0.0 | 0.3 |
| Lymph node | 0.0 | 0.0 |
| Colorectal | 0.0 | 0.0 |
| Stomach | 0.2 | 0.0 |
| Small intestine | 0.2 | 0.3 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.2 | 0.0 |
| Colon ca. CaCo-2 | 0.0 | 0.0 |
| 83219 CC Well to Mod Diff | 0.1 | 0.0 |
| (ODO3866) | | |
| Colon ca. HCC-2998 | 0.0 | 0.4 |
| Gastric ca.* (liver met) NCI-N87 | 0.0 | 0.0 |
| Bladder | 0.2 | 1.0 |
| Trachea | 0.0 | 0.3 |
| Kidney | 2.8 | 0.9 |
| Kidney (fetal) | 1.6 | 0.3 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.4 | 0.5 |
| Liver (fetal) | 0.1 | 0.5 |
| Liver ca. (hepatoblast) HepG2 | 0.0 | 0.0 |
| Lung | 0.0 | 0.2 |
| Lung (fetal) | 0.0 | 0.0 |
| Lung ca. (small cell) LX-1 | 0.0 | 0.0 |
| Lung ca. (small cell) NCI-H69 | 0.0 | 0.0 |
| Lung ca. (s. cell var.) SHP-77 | 0.0 | 0.0 |
| Lung ca. (large cell) NCI-H460 | 0.0 | 0.0 |
| Lung ca. (non-sm. cell) A549 | 0.0 | 0.0 |
| Lung ca. (non-s. cell) NCI-H23 | 0.5 | 0.3 |
| Lung ca (non-s. cell) HOP-62 | 0.0 | 0.0 |
| Lung ca. (non-s. cl) NCI-H522 | 8.1 | 0.3 |
| Lung ca. (squam.) SW 900 | 0.2 | 0.0 |
| Lung ca. (squam.) NCI-H596 | 0.0 | 0.0 |
| Mammary gland | 0.2 | 0.0 |
| Breast ca.* (pl. effusion) MCF-7 | 0.0 | 0.0 |
| Breast ca.* (pl. ef) MDA-MB-23 1 | 0.0 | 0.0 |
| Breast ca.* (pl. effusion) T47D | 0.1 | 0.0 |
| Breast ca. BT-549 | 0.2 | 0.2 |
| Breast ca. MDA-N | 0.0 | 0.0 |
| Ovary | 0.8 | 0.8 |
| Ovarian ca. OVCAR-3 | 0.0 | 0.0 |
| Ovarian ca. OVCAR-4 | 0.0 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca.* (ascites) SK-OV-3 | 0.0 | 0.0 |
| Uterus | 1.0 | 0.4 |
| Placenta | 0.2 | 0.0 |
| Prostate | 0.2 | 0.0 |
| Prostate ca.* (bone met) PC-3 | 0.0 | 0.0 |
| Testis | 1.1 | 1.4 |
| Melanoma Hs688(A).T | 0.0 | 0.2 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 0.7 | 0.3 |

TABLE 22
Panel 2D Summary

| | Relative | Relative |
|---|---|---|
| | Expression (%) | Expression (%) |
| | 2Dtm2345f_{—} | 2dtm4268t_{—} |
| Tissue Name | ag1508 | ag2454 |
| Normal Colon GENPAK 061003 | 2.2 | 9.3 |
| 83219 CC Well to Mod Diff (ODO3866) | 0.1 | 0.5 |
| 83220 CC NAT (ODO3866) | 1.4 | 5.6 |
| 83221 CC Gr.2 rectosigmoid (ODO3868) | 0.0 | 0.5 |
| 83222 CC NAT (ODO3868) | 0.6 | 1.7 |
| 83235 CC Mod Diff (ODO3920) | 0.0 | 0.0 |
| 83236 CC NAT (ODO3920) | 1.1 | 2.9 |
| 83237 CC Gr.2 ascend colon (ODO3921) | 0.1 | 0.7 |
| 83238 CC NAT (ODO3921) | 0.6 | 5.8 |
| 83241 CC from Partial Hepatectomy (ODO4309) | 0.3 | 0.0 |
| 83242 Liver NAT (ODO4309) | 2.4 | 6.7 |
| 87472 Colon mets to lung (OD04451-01) | 0.2 | 0.0 |
| 87473 Lung NAT (OD04451-02) | 0.4 | 0.4 |
| Normal Prostate Clontech A+ 6546-1 | 3.3 | 5.6 |
| 84140 Prostate Cancer (OD04410) | 3.4 | 4.8 |
| 84141 Prostate NAT (OD04410) | 0.5 | 5.4 |
| 87073 Prostate Cancer (OD04720-01) | 0.3 | 1.9 |
| 87074 Prostate NAT (OD04720-02) | 2.6 | 7.0 |
| Normal Lung GENPAK 061010 | 0.7 | 0.0 |
| 83239 Lung Met to Muscle (ODO4286) | 0.3 | 0.3 |
| 83240 Muscle NAT (ODO4286) | 100.0 | 100.0 |
| 84136 Lung Malignant Cancer (OD03126) | 0.3 | 0.0 |
| 84137 Lung NAT (OD03126) | 0.4 | 0.0 |
| 84871 Lung Cancer (OD04404) | 0.0 | 0.0 |
| 84872 Lung NAT (OD04404) | 0.3 | 0.0 |
| 84875 Lung Cancer (OD04565) | 0.0 | 0.4 |
| 84876 Lung NAT (OD04565) | 0.8 | 2.0 |
| 85950 Lung Cancer (OD04237-01) | 0.2 | 2.1 |
| 85970 Lung NAT (OD04237-02) | 0.5 | 0.0 |
| 83255 Ocular Mel Met to Liver (ODO4310) | 1.3 | 3.6 |
| 83256 Liver NAT (ODO4310) | 3.2 | 11.7 |
| 84139 Melanoma Mets to Lung (OD04321) | 0.0 | 0.0 |
| 84138 Lung NAT (OD04321) | 0.6 | 0.4 |
| Normal Kidney GENPAK 061008 | 18.8 | 27.9 |
| 83786 Kidney Ca, Nuclear grade 2 (OD04338) | 7.5 | 6.1 |
| 83787 Kidney NAT (OD04338) | 6.0 | 16.7 |
| 83788 Kidney Ca Nuclear grade 1/2 (OD04339) | 11.3 | 6.6 |
| 83789 Kidney NAT (OD04339) | 14.2 | 30.6 |
| 83790 Kidney Ca, Clear cell type (OD04340) | 2.5 | 4.5 |
| 83791 Kidney NAT (OD04340) | 11.4 | 33.9 |
| 83792 Kidney Ca, Nuclear grade 3 (OD04348) | 0.9 | 0.0 |
| 83793 Kidney NAT (OD04348) | 9.3 | 32.5 |
| 87474 Kidney Cancer (OD04622-01) | 0.4 | 0.0 |
| 87475 Kidney NAT (OD04622-03) | 1.7 | 1.0 |
| 85973 Kidney Cancer (OD04450-01) | 6.2 | 4.2 |
| 85974 Kidney NAT (OD04450-03) | 6.1 | 16.5 |
| Kidney Cancer Clontech 8120607 | 0.9 | 6.0 |
| Kidney NAT Clontech 8120608 | 11.3 | 3.5 |
| Kidney Cancer Clontech 8120613 | 3.6 | 2.5 |
| Kidney NAT Clontech 8120614 | 11.0 | 12.5 |
| Kidney Cancer Clontech 9010320 | 0.7 | 2.1 |
| Kidney NAT Clontech 9010321 | 12.0 | 4.8 |
| Normal Uterus GENPAK 061018 | 2.8 | 1.8 |
| Uterus Cancer GENPAK 064011 | 0.6 | 2.0 |
| Normal Thyroid Clontech A+6570-1 | 15.1 | 25.5 |
| Thyroid Cancer GENPAK 064010 | 7.1 | 8.3 |
| Thyroid Cancer INVITROGEN A302152 | 0.9 | 0.0 |
| Thyroid NAT INVITROGEN A3O2153 | 3.1 | 10.7 |
| Normal Breast GENPAK 061019 | 0.3 | 5.0 |
| 84877 Breast Cancer (OD04566) | 0.0 | 0.0 |
| 85975 Breast Cancer (OD04590-01) | 0.2 | 0.0 |
| 85976 Breast Cancer Mets (OD04590-03) | 0.7 | 1.7 |
| 87070 Breast Cancer Metastasis (OD04655-05) | 0.0 | 0.0 |
| GENPAK Breast Cancer 064006 | 0.2 | 0.7 |
| Breast Cancer Res. Gen. 1024 | 0.1 | 0.0 |
| Breast Cancer Clontech 9100266 | 0.4 | 0.0 |
| Breast NAT Clontech 9100265 | 0.3 | 0.0 |
| Breast Cancer INVITROGEN A209073 | 0.2 | 0.0 |
| Breast NAT INVITROGEN A2090734 | 0.0 | 2.8 |
| Normal Liver GENPAK 061009 | 1.6 | 8.9 |
| Liver Cancer GENPAK 064003 | 0.9 | 0.0 |
| Liver Cancer Research Genetics RNA 1025 | 1.1 | 3.6 |
| Liver Cancer Research Genetics RNA 1026 | 1.0 | 0.0 |
| Paired Liver Cancer Tissue Research Genetics RNA 6004-T | 2.3 | 5.3 |
| Paired Liver Tissue Research Genetics RNA 6004-N | 0.3 | 2.7 |
| Paired Liver Cancer Tissue Research Genetics RNA 6005-T | 0.7 | 3.0 |
| Paired Liver Tissue Research Genetics RNA 6005-N | 1.6 | 5.8 |
| Normal Bladder GENPAK 061001 | 0.9 | 0.5 |
| Bladder Cancer Research Genetics RNA 1023 | 0.0 | 0.7 |
| Bladder Cancer INVITROGEN A302173 | 0.1 | 0.0 |
| 87071 Bladder Cancer (OD04718-01) | 0.2 | 3.6 |
| 87072 Bladder Normal Adjacent (OD04718-03) | 2.9 | 4.1 |
| Normal Ovary Res. Gen. | 1.1 | 0.6 |
| Ovarian Cancer GENPAK 064008 | 0.3 | 2.5 |
| 87492 Ovary Cancer (OD04768-07) | 0.0 | 2.2 |
| 87493 Ovary NAT (OD04768-08) | 0.2 | 4.0 |
| Normal Stomach GENPAK 061017 | 0.9 | 2.8 |
| Gastric Cancer Clontech 9060358 | 0.3 | 0.0 |
| NAT Stomach Clontech 9060359 | 0.3 | 0.0 |
| Gastric Cancer Clontech 9060395 | 1.3 | 2.9 |
| NAT Stomach Clontech 9060394 | 0.4 | 1.0 |
| Gastric Cancer Clontech 9060397 | 0.4 | 0.0 |
| NAT Stomach Clontech 9060396 | 0.0 | 0.0 |
| Gastric Cancer GENPAK 064005 | 0.5 | 12.2 |

TABLE 23
Panel 4D

| | Relative | | Relative |
|---|---|---|---|
| | Expression | | Expression |
| | (%) | | (%) |
| | 4dtm4269t_{—} | | 4dtm4269t_{—} |
| Tissue Name | ag2454 | Tissue Name | ag2454 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HiUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | 0.0 |
| 93568_primary Th1_anti- | 0.0 | 93584_Lung Microvascular | 0.0 |
| CD28/anti-CD3 | | Endothelial Cells_TNFa (4 | |
| | | ng/ml) and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | |
| CD28/anti-CD3 | | endothelium_TNFa (4 ng/ml) | 0.0 |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml) ** | |
| 93566_primary Th2_resting dy | 0.0 | 93347_Small Airway | 0.0 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 0.0 |
| CD28/anti-CD3 | | | |
| 93353_chronic CD8 | 0.0 | 93108_astrocytes_TNFa (4 | 0.0 |
| Lymphocytes 2ry_resting dy 4- | | ng/ml) and IL1b (1 ng/ml) | |
| 6 in IL-2 | | | |
| 93574_chronic CD8 | 2.5 | 92666_KU-812 | 0.0 |
| Lymphocytes 2ry activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg ** | |
| 93788_LAK cells_IL-2 | 8.2 | 93791_Liver Cirrhosis | 0.0 |
| 93787_LAK cells_IL-2 + IL-12 | 0.0 | 93792_Lupus Kidney | 0.0 |
| 93789_LAK cells_IL-2+ IFN | 0.0 | 93577_NCI-H292 | 0.0 |
| gamma | | | |
| 93790_LAK cells_IL-2 + IL-18 | 0.0 | 93358_NCI-H292 IL-4 | 0.0 |
| 93104_LAK | 7.1 | 93360_NCI-H292_IL-9 | 0.0 |
| cells_PMA/ionomycin and IL- | | | |
| 18 | | | |
| 93578_NK Cells IL-2_resting | 0.0 | 93359_NCI-H292_IL-13 | 0.0 |
| 93109_Mixed Lymphocyte | 8.9 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 14.5 |
| (PBMCs)_resting | | Fibroblast_none | |
| 93113_Mononuclear Cells | 0.0 | 93253_Normal Human Lung | 5.0 |
| (PBMCs)_PWM | | Fibroblast_TNFa (4 ng/ml) and | |
| | | IL-lb (1 ng/ml) | |
| 93114_Mononuclear Cells | 0.0 | 93257_Normal Human Lung | 1.6 |
| (PBMCs)PHA-L | | Fibroblast_IL-4 | |
| 93249_Ramos (B cell)_none | 0.0 | 93256_Normal Human Lung | 9.3 |
| | | Fibroblast_IL-9 | |
| 93250_Ramos(B | 0.0 | 93255_Normal Human Lung | 18.2 |
| cell)_ionomycin | | Fibroblast_IL-13 | |
| 93349_B lymphocytes_PWM | 0.0 | 93258_Normal Human Lung | 4.9 |
| | | Fibroblast_IFN gamma | |
| 93350_B lymphoytes_CD40L | 0.0 | 93106_Dermal Fibroblasts | 19.9 |
| and IL-4 | | CCD1070_resting | |
| 92665_EOL-1 | 0.0 | 93361_Dermal Fibroblasts | 1.7 |
| (Eosinophil)_dbcAMP | | CCD1070_TNF alpha 4 ng/ml | |
| differentiated | | | |
| 93248_EOL-1 | 0.0 | 93105_Dermal Fibroblasts | 12.2 |
| (Eosinophil)_dbcAMP/PMA | | CCD1070_IL-1 beta 1 ng/ml | |
| ionomycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 5.3 |
| 100 ng/ml | | | |
| 93775_Dendritic Cells_anti- | 0.0 | 93259_IBD Colitis 1** | 0.0 |
| CD40 | | | |
| 93774_Monocytes_resting | 0.0 | 93260_IBD Colitis 2 | 0.0 |
| 93776_Monocytes LPS 50 | 0.0 | 93261_IBD Crohns | 9.5 |
| ng/ml | | | |
| 93581_Macrophages_resting | 10.7 | 735010_Colon_normal | 7.7 |
| 93582_Macrophages_LPS 100 | 0.0 | 735019_Lung_none | 0.0 |
| ng/ml | | | |
| 93098_HUVEC | 0.0 | 64028-1_Thymus _none | 100.0 |
| (Endothelial)_none | | | |
| 93099_HUVIEC | 0.0 | 64030-1_Kidney_none | 0.0 |
| (Endothelial)_starved | | | |

TABLE 24
Panel 4.1D

| | Relative | | Relative |
|---|---|---|---|
| | Expression (%) | | Expression % |
| | 4.1dx4tm5996f | | 4.1dx4tm5996f |
| Tissue Name | ag2284 at | Tissue Name | ag2284 at |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 1.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.7 | 93101_HUVEC | 0.0 |
| day 4-6 in IL-2 | | (Endothelial)_TNF alpha + IL4 | |
| 93572_Secondary Th2_resting | 0.5 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 | |
| | | ng/ml) and IL1b (1 ng/ml) | |
| 93569_primary Th2_anti- | 0.7 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 1.0 |
| 4-6 in IL-2 | | epithelium_TNFa (4 ng/ml) and | |
| | | IL1b (1 ng/ml) ** | |
| 93566_primary Th2_resting dy | 0.0 | 93347_Small Airway | 0.0 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 7.5 | 92668_Coronery Artery | 0.0 |
| lymphocyte_anti-CD28/anti- | | SMC resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti | 0.0 | 93107_astrocytes_resting | 1.9 |
| CD28/anti-CD3 | | | |
| 93353_chronic CD8 | 0.0 | 93108_astrocytes_TNFa (4 | 3.2 |
| Lymphocytes 2ry_resting dy 4- | | ng/ml) and IL1b (1 ng/ml) | |
| 6 in IL-2 | | | |
| 93574_chronic CD8 | 0.0 | 92666_KU-812 | 0.0 |
| Lymphocytes 2ry_activated | | (Basophul)_resting | |
| CD3/CD28 | | | |
| 93354_CD4_none | 0.0 | 92667_KU-812 | 0.9 |
| | | (Basophil) PMA/ionoycin | |
| 93252_Secondary | 1.2 | 93579_CCD1106 | 0.0 |
| Th1/Th2/Tr1_anti-CD95 CH11 | | (Keratinocytes)_none | |
| 93103_LAK cells_resting | 0.8 | 93580_CCD1106 | 0.0 |
| | | (Keratinocytes)_TNFa and | |
| | | IFNg ** | |
| 93788_LAK cells_IL-2 | 0.0 | 93791_Liver Cirrhosis | 2.2 |
| 93787_LAK cells_IL-2 + IL-12 | 0.4 | 93577_NCI-H292 | 0.8 |
| 93789_LAK cells_IL-2 + IFN | 0.0 | 93358_NCI-H292_IL-4 | 0.0 |
| gamma | | | |
| 93790_LAK cells_IL-2 + IL-18 | 0.0 | 93360_NCI-H292_IL-9 | 0.0 |
| 93104_LAK | 1.5 | 93359_NCI-H292_IL-13 | 0.0 |
| cells_PMA/ionomycin and IL-18 | | | |
| 93578_NK Cells IL-2_resting | 1.3 | 93357 NCI-H292_IFN gamma | 0.0 |
| 93109_Mixed Lymphocyte | 1.3 | 93777_HPAEC_- | 0.0 |
| Reaction_Two Way MLR | | | |
| 93110_Mixed Lymphocyte | 1.8 | 93778_HPAEC_IL-1 beta/TNA | 0.0 |
| Reaction_Two Way MLR | | alpha | |
| 93111_Mixed Lymphocyte | 0.0 | 93254_Normal Human Lung | 28.0 |
| Reaction_Two Way MLR | | Fibroblast_none | |
| 93112_Mononuclear Cells | 0.0 | 93253_Normal Human Lung | 4.7 |
| (PBMCs)_resting | | Fibroblast_TNFa (4 ng/ml) and | |
| | | IL-1b (1 ng/ml) | |
| 93113_Mononuclear Cells | 0.9 | 93257_Normal Human Lung | 19.3 |
| (PBMCs)_PWM | | Fibroblast_1L4 | |
| 93114_Mononuclear Cells | 0.0 | 93256_Normal Human Lung | 32.2 |
| (PBMCs)_PHA-L | | Fibroblast_IL-9 | |
| 93249_Ramos (B cell)_none | 0.0 | 93255_Normal Human Lung | 11.4 |
| | | Fibroblast_IL-13 | |
| 93250_Ramos (B | 0.0 | 93258_Normal Human Lung | 9.9 |
| cell)_ionomycin | | Fibroblast_IFN gamma | |
| 93349_B lymphocytes_PWM | 0.8 | 93106_Dermal Fibroblasts | 43.1 |
| | | CCD1070_resting | |
| 93350_B lymphoytes_CD40L | 0.0 | 93361_Dermal Fibroblasts | 30.9 |
| and IL-4 | | CCD1070_TNF alpha 4 ng/ml | |
| 92665_EOL-1 | 0.0 | 93105_Dermal Fibroblasts | 7.4 |
| (Eosinophil)_dbcAMP | | CCD1070_IL-1 beta 1 ng/ml | |
| differentiated | | | |
| 93248_EOL-1 | 0.0 | 93772_dermal fibroblast_IFN | 5.8 |
| (Eosinophil)_dbcAMP/PMAion | | gamma | |
| omycin | | | |
| 93356_Dendritic Cells_none | 0.0 | 93771_dermal fibroblast_IL-4 | 38.3 |
| 93355_Dendritic Cells_LPS | 0.5 | 93892_Dermal fibroblasts_{—} | 24.7 |
| 100 ng/ml | | none | |
| 93775_Dendritic Cells_anti- | 0.9 | 99202_Neutrophils_TNFa + | 0.0 |
| CD40 | | LPS | |
| 93774_Monocytes_resting | 0.0 | 99203_Neutrophils_none | 0.0 |
| 93776_Monocytes_LPS 50 | 2.4 | 735010_Colon_normal | 1.0 |
| ng/ml | | | |
| 93581_Macrophages_resting | 8.9 | 735019_Lung_none | 7.3 |
| 93582_Macrophages_LPS 100 | 0.0 | 64028-1_Thymus_none | 3.1 |
| ng/ml | | | |
| 93098_HUVEC | 0.0 | 64030-1_Kidney_none | 100.0 |
| (Endothelial)_none | | | |
| 93099_HUVEC | 0.0 | | |
| (Endothelial)_starved | | | |

Panel 1.2 Summary:

Ag1508 The expression of the NOV3A gene is highest in a sample derived from skeletal muscle (CT=19.5). Thus, this gene could be used to distinguish skeletal muscle from other tissues. Expression of the NOV3a gene is also high in kidney (CT=23). The NOV3a gene product is highly homologous to mitsugumin 29. Expression of the NOV3a gene in skeletal muscle and kidney is consistent with what has been observed for the mitsugumin29 gene (Ref. 1). Interestingly, mitsugumin29-deficient mice are slightly reduced in body weight and appear to have abnormal skeletal muscle (Ref. 2). Therefore, the NOV3a gene product may useful as a small molecule drug target in the treatment of obesity and/or skeletal muscle diseases, including muscular dystrophy, Lesch-Nyhan syndrome, and myasthenia gravis. The NOV3a gene is also more moderately expressed in other metabolically relevant tissues including heart, adrenal gland, pancreas, thyroid, pituitary gland, and liver (CT values from 29-32).

The NOV3a gene is moderately expressed in the brain in at least the thalamus, hippocampus, cerebellum, amygdala and is highly expressed in the cerebral cortex, suggesting that this gene product has functional significance in the CNS. The NOV3a gene product is highly homologous to mitsugumin, a member of the synaptophysin family. Mitsugumin is expressed on intracellular membranes, including synaptic vesicles and the triad junction that mediates intracellular calcium release induced by depolarization. Studies have shown that schizophrenia, which is known to involve abnormal neuronal signaling in the cerebral cortex, involves the abnormal expression of synaptic genes, in particular presynaptic genes (Ref. 3-4). Synaptic vesicle mobilization and calcium response to depolarization are pre- and post-synaptic signaling events, potentially involving the NOV3a gene. Therefore, the NOV3a gene product and agents that modulate its function may be useful in treating diseases of the CNS, such as schizophrenia. Synaptic function is also compromised in other diseases such as epilepsy, stroke, Alzheimer's disease, as well as other neurodegenerative diseases. Thus, the NOV3a gene product and agents that modulate its function may be useful in treating these CNS diseases as well.

Panel 1.3D Summary:

Ag2284/Ag2454 Results from experiments using two different probe/primer sets are in reasonable agreement. These results are also consistent with what is observed in Panel 1.2 Ag2284 The NOV3a gene is most highly expressed in fetal skeletal muscle (CT=26.3) and adult skeletal muscle (CT=26.4). Much lower but significant expression is also detected in adipose, testis, uterus, ovary, kidney, heart, thyroid and adrenal gland (CTs=31-33). Ag2454 The expression of the NOV3a gene in this experiment is highest and almost exclusive to fetal skeletal muscle (CT=29.5). However, significant expression is also seen in adult skeletal muscle (CT=33.4). Thus, expression of the NOV3A gene could be used to distinguish skeletal muscle from other tissues. In addition, therapeutic modulation of this gene or gene product, through replacement therapy, could be used as a regenerative therapy for muscle disease.

Panel 2D Summary: Ag1508/Ag2454 Results from experiments using two different probe/primer sets are in reasonable agreement. Expression of the NOV3a gene in Panel 2 is highest in a sample of muscle tissue adjacent to a metastatic cancer. In addition, there is moderate expression in normal kidney tissue (CT 30-31) when compared to malignant kidney. Thus, the expression of this gene could be used to distinguish normal kidney tissue from malignant kidney tissue. In addition, therapeutic modulation of the NOV3a gene product is of use in the treatment of kidney cancer.

Panel 4D Summary: Ag2454 Significant expression of the NOV3a gene in this panel is limited to thymus (CT=33). The NOV3a gene encodes a protein with homology to mitsugumin, a member of the synaptophysin family. Synaptophysin is also expressed in the thymus and is thought to be involved in secretory activities and perhaps in specialized endoplasmic reticulum systems (Ref. 5). Therefore, therapuetic drugs designed against the NOV3a gene product may be important for regulating the function of the thymus. Regulating thymus function may in turn regulate T cell development and immune function.

Panel 4.1D Summary: Ag2284 Significant expression in this panel is limited to kidney. This observation is consistent with what was observed in other panels. Furthermore, the homologous mitsugumin29 gene is also expressed in the kidney and is thought to be involved in secretory activities and perhaps in specialized endoplasmic reticulum systems (Ref. 1). Therefore, therapuetic drugs designed against the NOV3a gene product may be important for regulating the function of the kidney.

NOV4

Expression of the NOV4 gene was assessed using the primer-probe sets Gpcr38, Ag998, and Gper10, described in Tables 25, 26 and 27. Results from RTQ-PCR runs are shown in Tables 28, 29, 30, 31, 32, 33, and 34.

TABLE 25
Probe Name Gpcr38

| | | | | | |
|---|---|---|---|---|---|
| | | | | Start | |
| Primers | Sequences | TM | Length | Position | SEQ ID NO: |

| | | | | |
|---|---|---|---|---|
| Forward | 5′-TGTTGGTACTGCTGTTAAGTTGCA-3′ | 24 | 393 | 84 |
| Probe | FAM-5′-TCTCCAGGGTGAGCTGCTCCAAGC- | 24 | 419 | 85 |
| | 3′-TAMRA | | | |
| Reverse | 5′-AGGGCATTCAGTGGGCTTCT-3′ | 20 | 445 | 86 |

TABLE 26
Probe Name Ag998

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-CAATATGCCTGTGTATGCCTTT-3′ | 59 | 22 | 193 | 87 |
| Probe | TET-5′- | 64.2 | 26 | 215 | 88 |
| | AAAAGATTGTTCCACCTGAAACACCT-3′ | | | | |
| | TAMRA | | | | |
| Reverse | 5′-TCCAGTAAAGGCCAATAGTCAA-3′ | 58.8 | 22 | 246 | 89 |

TABLE 27
Probe Name Gpcr10 (there is a single base mismatch in rev primer)

| | | | | | |
|---|---|---|---|---|---|
| | | | | Start | |
| Primers | Sequences | TM | Length | Position | SEQ ID NO: |

| | | | | |
|---|---|---|---|---|
| Forward | 5′-ACAGCAGTACCAACAGAAGCCC-3′ | 22 | 119 | 90 |
| Probe | FAM-5′-TCCCACCTCCGCAGCCTCATCA- | 22 | 143 | 91 |
| | 3′-TAMRA | | | |
| Reverse | 5′-ATATTGACATGCTTCAGATGCAGG-3′ | 24 | 166 | 92 |

TABLE 28
Panel 1

| | Relative | | Relative |
|---|---|---|---|
| | Expression % | | Expression % |
| | tm597f | | tm597f_ |
| Tissue Name | gpcr10 | Tissue Name | gpcr10 |
| Endothelial cells | 0.0 | Kidney (fetal) | 0.0 |
| Endothelial cells (treated) | 0.0 | Renal ca. 786-0 | 0.0 |
| Pancreas | 0.0 | Renal ca. A498 | 0.0 |
| Pancreatic ca. CAPAN 2 | 0.0 | Renal Ca. RXF 393 | 0.0 |
| Adipose | 62.8 | Renal ca. ACHN | 0.0 |
| Adrenal gland | 0.0 | Renal ca. UO-31 | 12.9 |
| Thyroid | 19.5 | Renal ca. TK-10 | 7.1 |
| Salivary gland | 0.0 | Liver | 0.0 |
| Pituitary gland | 15.5 | Liver (fetal) | 0.0 |
| Brain (fetal) | 27.4 | Liver ca. (hepatoblast) HepG2 | 0.0 |
| Brain (whole) | 11.6 | Lung | 0.0 |
| Brain (amygdala) | 29.9 | Lung (fetal) | 0.0 |
| Brain (cerebellum) | 1.9 | Lung ca. (small cell) LX-1 | 0.0 |
| Brain (hippocampus) | 30.1 | Lung ca. (small cell) NCI-H69 | 100.0 |
| Brain (substantia nigra) | 10.4 | Lung ca. (s.cell var.) SHP-77 | 2.6 |
| Brain (thalamus) | 32.5 | Lung ca. (large cell) NCI-H460 | 2.8 |
| Brain (hypothalamus) | 3.7 | Lung ca. (non-sm. cell) A549 | 12.2 |
| Spinal cord | 2.8 | Lung Ca. (non-s. cell) NCI-H23 | 0.0 |
| CNS ca. (glio/astro) U87-MG | 32.5 | Lung ca (non-s. cell) HOP-62 | 1.3 |
| CNS ca. (glio/astro) U-118-MG | 0.0 | Lung ca. (non-s. cl) NCI-H522 | 0.0 |
| CNS ca. (astro) SW1783 | 0.0 | Lung ca. (squam.) SW 900 | 25.7 |
| CNS ca.* (neuro; met) SK- | 62.8 | Lung Ca. (squam.) NCI-H596 | 86.5 |
| N-AS | | | |
| CNS ca. (astro) SF-539 | 0.0 | Mammary gland | 0.0 |
| CNS ca. (astro) SNB-75 | 20.9 | Breast ca.* (p1. effusion) | 0.0 |
| | | MCF-7 | |
| CNS ca. (glio) SNB-19 | 69.3 | Breast ca.* (p1. ef) MDA-MB- | 0.0 |
| | | 231 | |
| CNS ca. (glio) U251 | 19.3 | Breast ca.* (p1. effusion) | 0.0 |
| | | T47D | |
| CNS Ca. (glio) SF-295 | 61.1 | Breast Ca. BT-549 | 21.3 |
| Heart | 0.0 | Breast ca. MDA-N | 4.9 |
| Skeletal muscle | 0.0 | Ovary | 4.4 |
| Bone marrow | 0.0 | Ovarian ca. OVCAR-3 | 13.8 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 42.0 |
| Colon (ascending) | 5.7 | Ovarian Ca. IGROV-1 | 0.0 |
| Stomach | 0.1 | Ovanan ca.* (ascites) SK-OV-3 | 0.0 |
| Small intestine | 0.0 | Uterus | 0.6 |
| Colon ca. SW480 | 0.0 | Placenta | 0.0 |
| Colon ca.* (SW480 met) SW620 | 0.0 | Prostate | 0.0 |
| Colon Ca. HT29 | 0.0 | Prostate ca.* (bone met) PC-3 | 21.9 |
| Colon Ca. HCT-116 | 0.0 | Testis | 20.7 |
| Colon ca. CaCo-2 | 0.0 | Melanoma Hs688 (A). T | 0.0 |
| Colon Ca. HCT-15 | 0.0 | Melanoma* (met) Hs688 (B). T | 0.0 |
| Colon ca. HCC-2998 | 0.0 | Melanoma UACC-62 | 0.0 |
| Gastric ca.* (liver met) NCI- | 0.0 | Melanoma M14 | 6.9 |
| N87 | | | |
| Bladder | 0.2 | Melanoma LOX IMVI | 0.0 |
| Trachea | 0.0 | Melanoma* (met) SK-MEL-5 | 0.0 |
| Kidney | 1.8 | Melanoma SK-MEL-28 | 0.0 |

TABLE 29
Panel 1.1

| | | | Relative |
|---|---|---|---|
| | Relative Expression(%) | | Expression(%) |
| | 1.1tm611f_{—} | 1.1tm643f_{—} | 1.1tm769f_{—} |
| Tissue Name | gpcr10 | gpcr10 | gpcr38 |
| Adipose | 12.0 | 7.5 | 3.2 |
| Adrenal gland | 0.0 | 0.8 | 1.2 |
| Bladder | 0.2 | 1.1 | 1.7 |
| Brain (amygdala) | 20.0 | 9.5 | 6.2 |
| Brain (cerebellum) | 19.6 | 8.5 | 19.8 |
| Brain (hippocampus) | 27.0 | 18.8 | 14.0 |
| Brain (substantia nigra) | 13.8 | 7.1 | 13.1 |
| Brain (thalamus) | 27.7 | 10.4 | 16.7 |
| Cerebral Cortex | 95.9 | 51.4 | 57.4 |
| Brain (fetal) | 53.2 | 19.5 | 29.1 |
| Brain (whole) | 54.0 | 24.3 | 26.8 |
| CNS ca. (glio/astro) U-118-MG | 0.0 | 0.0 | 0.0 |
| CNS ca. (astro) SF-539 | 0.0 | 0.0 | 0.0 |
| CNS ca. (astro) SNB-75 | 21.6 | 7.9 | 11.4 |
| CNS ca. (astro) SW1783 | 0.0 | 0.0 | 0.0 |
| CNS ca. (glio) U251 | 25.2 | 9.5 | 12.9 |
| CNS ca. (glio) SF-295 | 77.4 | 39.2 | 71.7 |
| CNS ca. (glio) SNB-19 | 64.2 | 21.6 | 43.8 |
| CNS ca. (glio/astro) U87-MG | 32.8 | 12.2 | 20.2 |
| CNS ca.* (neuro; met) SK-N-AS | 79.0 | 35.8 | 41.8 |
| Mammary gland | 0.0 | 0.1 | 0.6 |
| Breast ca. BT-549 | 15.3 | 0.0 | 7.9 |
| Breast ca. MDA-N | 1.8 | 3.6 | 4.4 |
| Breast ca.* (p1. effusion) T47D | 0.0 | 0.2 | 0.7 |
| Breast ca.* (p1. effusion) MCF-7 | 0.0 | 0.0 | 0.0 |
| Breast ca.* (p1. ef) MDA-MB-231 | 0.0 | 0.0 | 0.0 |
| Small intestine | 0.0 | 0.7 | 0.7 |
| Colorectal | 0.0 | 0.0 | 0.2 |
| Colon ca. HT29 | 0.0 | 0.0 | 0.3 |
| Colon ca. CaCo-2 | 0.0 | 0.4 | 0.6 |
| Colon ca. HCT-15 | 0.0 | 0.0 | 0.8 |
| Colon ca. HCT-116 | 0.0 | 0.0 | 0.0 |
| Colon ca. HCC-2998 | 0.0 | 0.0 | 0.0 |
| Colon ca. SW480 | 0.0 | 0.0 | 0.0 |
| Colon ca.* (SW480 met) SW620 | 0.0 | 0.0 | 0.0 |
| Stomach | 3.4 | 4.3 | 2.5 |
| Gastric ca.* (liver met) NCI-N87 | 0.0 | 0.3 | 0.2 |
| Heart | 0.0 | 0.0 | 1.0 |
| Fetal Skeletal | 1.0 | 2.7 | 2.6 |
| Skeletal muscle | 0.0 | 0.0 | 0.1 |
| Endothelial cells | 0.0 | 0.0 | 0.0 |
| Heart (fetal) | 0.0 | 0.0 | 0.4 |
| Kidney | 5.7 | 3.6 | 11.6 |
| Kidney | 2.6 | 2.9 | 3.7 |
| Renal ca. 786-0 | 0.0 | 0.0 | 0.0 |
| Renal ca. A498 | 0.9 | 3.5 | 1.3 |
| Renal ca. ACHN | 0.0 | 0.8 | 0.9 |
| Renal ca. TK-10 | 6.3 | 5.1 | 4.6 |
| Renal ca. UO-31 | 17.3 | 8.8 | 10.4 |
| Renal ca. RXF 393 | 0.0 | 0.6 | 0.4 |
| Liver | 0.0 | 0.2 | 0.0 |
| Liver (fetal) | 0.0 | 0.0 | 0.0 |
| Liver ca. (hepatoblast) HepG2 | 0.0 | 0.0 | 0.0 |
| Lung | 0.0 | 0.0 | 0.1 |
| Lung (fetal) | 0.0 | 0.3 | 0.3 |
| Lung ca (non-s. cell) HOP-62 | 1.1 | 2.4 | 5.4 |
| Lung ca. (large cell) NCI-H460 | 0.0 | 2.5 | 1.4 |
| Lung ca. (non-s. cell) NCI-H23 | 0.0 | 0.0 | 0.0 |
| Lung ca. (non-s. d) NCI-H522 | 0.0 | 0.0 | 0.0 |
| Lung ca. (non-sm. cell) A549 | 5.0 | 4.8 | 7.1 |
| Lung ca. (s. cell var.) SHP-77 | 6.5 | 5.6 | 5.3 |
| Lung ca. (small cell) LX-1 | 0.0 | 0.0 | 0.0 |
| Lung ca. (small cell) NCI-H69 | 100.0 | 100.0 | 100.0 |
| Lung ca. (squam.) SW 900 | 9.3 | 7.3 | 9.0 |
| Lung ca. (sguam.) NCI-H596 | 77.4 | 41.2 | 55.9 |
| Lymph node | 0.0 | 0.1 | 0.0 |
| Spleen | 0.0 | 1.4 | 0.5 |
| Thymus | 0.0 | 0.9 | 0.1 |
| Ovary | 1.4 | 2.8 | 2.2 |
| Ovarian ca. IGROV-1 | 0.0 | 0.0 | 0.2 |
| Ovarian ca. OVCAR-3 | 14.3 | 9.9 | 6.1 |
| Ovarian ca. OVCAR-4 | 0.0 | 0.0 | 0.0 |
| Ovarian ca. OVCAR-5 | 0.0 | 2.3 | 2.5 |
| Ovarian ca. OVCAR-8 | 10.7 | 5.0 | 8.3 |
| Ovarian ca.* (ascites) SK-OV-3 | 0.0 | 0.8 | 1.1 |
| Pancreas | 1.7 | 4.4 | 6.3 |
| Pancreatic ca. CAPAN 2 | 0.0 | 0.0 | 0.0 |
| Pituitary gland | 4.2 | 4.8 | 4.7 |
| Placenta | 0.4 | 2.4 | 1.8 |
| Prostate | 0.0 | 0.7 | 1.3 |
| Prostate ca.* (bone met) PC-3 | 13.3 | 7.3 | 10.1 |
| Salivary gland | 0.0 | 0.1 | 1.2 |
| Trachea | 0.0 | 1.1 | 0.6 |
| Spinal cord | 1.3 | 8.1 | 2.8 |
| Testis | 16.4 | 9.9 | 5.2 |
| Thyroid | 0.0 | 0.0 | 14.9 |
| Uterus | 40.6 | 24.0 | 0.0 |
| Melanoma M14 | 4.5 | 5.2 | 5.5 |
| Melanoma LOX IMYI | 0.0 | 0.9 | 1.1 |
| Melanoma UACC-62 | 0.0 | 0.0 | 0.1 |
| Melanoma SK-MEL-28 | 34.9 | 12.6 | 20.7 |
| Melanoma* (met) SK-MEL-5 | 0.0 | 0.3 | 0.5 |
| Melanoma Hs688 (A). T | 0.0 | 0.0 | 0.0 |
| Melanoma* (met) Hs688 (B). T | 0.0 | 0.7 | 0.6 |

TABLE 30
Panel 1.3D

| | Relative | Relative |
|---|---|---|
| | Expression | Expression |
| | 1.3Dtm3184f_{—} | 1.3Dtm3393t_{—} |
| Tissue Name | Gpcr10 | ag998 |
| Liver adenocarcinoma | 0 | 0 |
| Pancreas | 1.7 | 0.8 |
| Pancreatic ca. CAPAN 2 | 0 | 0 |
| Adrenal gland | 1.4 | 0.7 |
| Thyroid | 5.3 | 6.6 |
| Salivary gland | 0 | 0.2 |
| Pituitary gland | 2.5 | 0.9 |
| Brain (fetal) | 11.4 | 10.7 |
| Brain (whole) | 12.6 | 10.4 |
| Brain (amygdala) | 13 | 13.8 |
| Brain (cerebellum) | 1.4 | 0.7 |
| Brain (hippocampus) | 43.2 | 51 |
| Brain (substantia nigra) | 1.2 | 0.9 |
| Brain (thalamus) | 15 | 9.7 |
| Cerebral Cortex | 100 | 100 |
| Spinal cord | 1.4 | 2.5 |
| CNS ca. (glio/astro) U87-MG | 9.3 | 6.1 |
| CNS ca. (glio/astro) U-118-MG | 0.4 | 0.2 |
| CNS ca. (astro) SW1783 | 0 | 0 |
| CNS ca.* (neuro; met) SK-N-AS | 25.5 | 20.4 |
| CNS ca. (astro) SF-539 | 0 | 0 |
| CNS ca. (astro) SNB-75 | 7.4 | 2.7 |
| CNS ca. (glio) SNB-19 | 16.3 | 16.6 |
| CNS ca. (glio) U251 | 8.5 | 6.6 |
| CNS ca. (glio) SF-295 | 39.8 | 27.4 |
| Heart (fetal) | 0.5 | 0.7 |
| Heart | 0.3 | 0 |
| Fetal Skeletal | 10.7 | 9.4 |
| Skeletal muscle | 0 | 0.3 |
| Bone marrow | 0 | 0 |
| Thymus | 1.1 | 0.4 |
| Spleen | 0.5 | 0.5 |
| Lymph node | 0.7 | 0 |
| Colorectal | 1.4 | 1.2 |
| Stomach | 2.7 | 1.4 |
| Small intestine | 0.6 | 0.4 |
| Colon ca. SW480 | 0 | 0 |
| Colon ca.* (SW480 met) SW620 | 0 | 0 |
| Colon ca. HT29 | 0 | 0 |
| Colon ca. HCT-116 | 0 | 0 |
| Colon ca. CaCo-2 | 0.7 | 0.2 |
| 83219 CC Well to Mod Diff | 0.7 | 0.4 |
| (ODO3866) | | |
| Colon ca. HCC-2998 | 0 | 0 |
| Gastric ca.* (liver met) NCI-N87 | 0 | 0 |
| Bladder | 0.4 | 0.6 |
| Trachea | 1.1 | 1.1 |
| Kidney | 0.4 | 0.5 |
| Kidney (fetal) | 2 | 0.9 |
| Renal ca. 786-0 | 0 | 0 |
| Renal ca. A498 | 1.8 | 1.3 |
| Renal ca. RXF 393 | 0.3 | 0.5 |
| Renal ca. ACHN | 0 | 0 |
| Renal ca. UO-31 | 2.7 | 1.2 |
| Renal ca. TK-10 | 1.1 | 1.8 |
| Liver | 0 | 0.2 |
| Liver (fetal) | 0.6 | 0 |
| Liver ca. (hepatoblast) HepG2 | 0.7 | 0 |
| Lung | 0 | 1 |
| Lung (fetal) | 0.4 | 0.5 |
| Lung ca. (small cell) LX-1 | 0 | 0 |
| Lung ca. (small cell) NCI-H69 | 79.6 | 73.7 |
| Lung ca. (s. cell var.) SHP-77 | 6.3 | 5.3 |
| Lung ca. (large cell) NCI-H460 | 0.4 | 0.2 |
| Lung ca. (non-sm. cell) A549 | 0.7 | 0.6 |
| Lung ca. (non-s. cell) NCI-H23 | 0 | 0.3 |
| Lung ca (non-s. cell) HOP-62 | 0 | 0.2 |
| Lung ca. (non-s.d) NCI-H522 | 1 | 0.2 |
| Lung ca. (squam.) SW 900 | 3.3 | 2.5 |
| Lung ca. (squam.) NCI-H596 | 15.3 | 9.7 |
| Mammary gland | 0.8 | 0 |
| Breast ca.* (p1. effusion) MCF-7 | 0 | 0 |
| Breast ca.* (p1. ef) MDA-MB-231 | 0 | 0 |
| Breast ca.* (p1. effusion) T47D | 0 | 0 |
| Breast ca. BT-549 | 9.6 | 8.2 |
| Breast ca. MDA-N | 1.8 | 0.9 |
| Ovary | 4.3 | 2.7 |
| Ovarian ca. OVCAR-3 | 1.8 | 1.6 |
| Ovarian ca. OVCAR-4 | 0 | 0 |
| Ovarian ca. OVCAR-5 | 0 | 0 |
| Ovarian ca. OVCAR-8 | 3.8 | 2.3 |
| Ovarian ca. IGROV-1 | 0 | 0 |
| Ovarian ca.* (ascites) SK-OV-3 | 0 | 0 |
| Uterus | 21.3 | 21 |
| Placenta | 0 | 0 |
| Prostate | 0.7 | 1.5 |
| Prostate ca.* (bone met) PC-3 | 3 | 1.3 |
| Testis | 9.8 | 6.9 |
| Melanoma Hs688 (A). T | 2.2 | 0.3 |
| Melanoma* (met) Hs688 (B). T | 2.2 | 0.8 |
| Melanoma UACC-62 | 0 | 0 |
| Melanoma M14 | 2.5 | 1.8 |
| Melanoma LOX IMVI | 1.1 | 0.9 |
| Melanoma* (met) SK-MEL-5 | 0 | 0 |
| Adipose | 0.3 | 0 |

TABLE 31
Panel 2D

| | Relative | Relative |
|---|---|---|
| | Expression | Expression |
| | 2Dtm3154f_{—} | 2Dtm3394t_ |
| Tissue Name | Gpcr10 | ag998 |
| Normal Colon GENPAK 061003 | 8.4 | 1.5 |
| 83219 CC Well to Mod Diff | 3.1 | 1.5 |
| (ODO3866) | | |
| 83220 CC NAT (ODO3866) | 3.7 | 1.5 |
| 83221 CC Gr.2 rectosigmoid | 1.3 | 0.5 |
| (ODO3868) | | |
| 83222 CC NAT (ODO3868) | 2.5 | 0.7 |
| 83235 CC Mod Diff (ODO3920) | 0.0 | 0.0 |
| 83236 CC NAT (ODO3920) | 3.9 | 2.7 |
| 83237 CC Gr.2 ascend colon | 1.0 | 0.0 |
| (ODO3921) | | |
| 83238 CC NAT (ODO3921) | 4.9 | 3.2 |
| 83241 CC from Partial Hepatectomy | 0.7 | 0.0 |
| (ODO4309) | | |
| 83242 Liver NAT (ODO4309) | 0.9 | 0.0 |
| 87472 Colon mets to lung (OD04451- | 0.0 | 1.2 |
| 01) | | |
| 87473 Lung NAT (OD04451-02) | 1.7 | 0.6 |
| Normal Prostate Clontech A+ 6546-1 | 3.1 | 2.0 |
| 84140 Prostate Cancer (OD04410) | 2.3 | 0.7 |
| 84141 Prostate NAT (OD04410) | 21.5 | 12.3 |
| 87073 Prostate Cancer (OD04720-01) | 3.3 | 2.1 |
| 87074 Prostate NAT (OD04720-02) | 6.7 | 6.7 |
| Normal Lung GENPAK 061010 | 2.8 | 1.4 |
| 83239 Lung Met to Muscle (ODO4286) | 11.2 | 11.8 |
| 83240 Muscle NAT (ODO4286) | 2.1 | 1.0 |
| 84136 Lung Malignant Cancer | 2.8 | 0.5 |
| (OD03126) | | |
| 84137 Lung NAT (OD03126) | 2.1 | 2.9 |
| 84871 Lung Cancer (OD04404) | 4.0 | 2.1 |
| 84872 Lung NAT (OD04404) | 1.7 | 0.0 |
| 84875 Lung Cancer (OD04565) | 0.0 | 0.8 |
| 84876 Lung NAT (OD04565) | 3.4 | 2.8 |
| 85950 Lung Cancer (OD04237-01) | 44.4 | 40.6 |
| 85970 Lung NAT (OD04237-02) | 0.6 | 0.5 |
| 83255 Ocular Mel Met to Liver | 24.3 | 15.8 |
| (ODO4310) | | |
| 83256 Liver NAT (ODO4310) | 0.0 | 0.0 |
| 84139 Melanoma Mets to Lung | 100.0 | 100.0 |
| (OD04321) | | |
| 84138 Lung NAT (OD04321) | 3.1 | 2.6 |
| Normal Kidney GENPAK 061008 | 16.3 | 21.6 |
| 83786 Kidney Ca, Nuclear grade 2 | 0.0 | 0.8 |
| (OD04338) | | |
| 83787 Kidney NAT (OD04338) | 9.9 | 14.0 |
| 83788 Kidney Ca Nuclear grade 1/2 | 0.0 | 0.0 |
| (OD04339) | | |
| 83789 KidneyNAT (OD04339) | 27.5 | 17.8 |
| 83790 Kidney Ca, Clear cell type | 2.3 | 1.6 |
| (OD04340) | | |
| 83791 Kidney NAT (OD04340) | 9.4 | 9.7 |
| 83792 Kidney Ca, Nuclear grade 3 | 0.7 | 0.0 |
| (OD04348) | | |
| 83793 Kidney NAT (OD04348) | 4.9 | 3.7 |
| 87474 Kidney Cancer (OD04622-01) | 1.3 | 0.0 |
| 87475 Kidney NAT (OD04622-03) | 3.0 | 1.9 |
| 85973 Kidney Cancer (OD04450-01) | 0.0 | 0.0 |
| 85974 Kidney NAT (OD04450-03) | 10.2 | 12.5 |
| Kidney Cancer Clontech 8120607 | 0.8 | 1.6 |
| Kidney NAT Clontech 8120608 | 2.7 | 0.5 |
| Kidney Cancer Clontech 8120613 | 1.3 | 0.0 |
| Kidney NAT Clontech 8120614 | 8.4 | 5.4 |
| Kidney Cancer Clontech 9010320 | 0.3 | 0.3 |
| Kidney NAT Clontech 9010321 | 10.4 | 7.3 |
| Normal Uterus GENPAK 061018 | 58.6 | 45.1 |
| Uterus Cancer GENPAK 064011 | 41.8 | 43.2 |
| Normal Thyroid Clontech A+ 6570-1 | 32.3 | 27.5 |
| Thyroid Cancer GENPAK 064010 | 0.0 | 0.5 |
| Thyroid Cancer INVITROGEN | 2.1 | 0.8 |
| A302152 | | |
| Thyroid NAT INVITROGEN A302153 | 18.4 | 13.4 |
| Normal Breast GENPAK 061019 | 2.9 | 0.0 |
| 84877 Breast Cancer (OD04566) | 1.3 | 0.6 |
| 85975 Breast Cancer (OD04590-01) | 3.6 | 0.9 |
| 85976 Breast Cancer Mets (OD04590- | 0.8 | 0.0 |
| 03) | | |
| 87070 Breast Cancer Metastasis | 0.9 | 0.4 |
| (OD04655-05) | | |
| GENPAK Breast Cancer 064006 | 0.9 | 1.1 |
| Breast Cancer Res. Gen. 1024 | 1.7 | 1.2 |
| Breast Cancer Clontech 9100266 | 2.0 | 3.5 |
| Breast NAT Clontech 9100265 | 1.2 | 0.7 |
| Breast Cancer INVITROGEN A209073 | 7.4 | 7.9 |
| Breast NAT INVITROGEN A2090734 | 2.5 | 1.6 |
| Normal Liver GENPAK 061009 | 0.0 | 0.9 |
| Liver Cancer GENPAK 064003 | 1.5 | 0.0 |
| Liver Cancer Research Genetics RNA | 0.7 | 0.0 |
| 1025 | | |
| Liver Cancer Research Genetics RNA | 0.0 | 0.6 |
| 1026 | | |
| Paired Liver Cancer Tissue Research | 0.0 | 0.5 |
| Genetics RNA 6004-T | | |
| Paired Liver Tissue Research Genetics | 2.6 | 1.5 |
| RNA 6004-N | | |
| Paired Liver Cancer Tissue Research | 0.8 | 0.5 |
| Genetics RNA 6005-T | | |
| Paired Liver Tissue Research Genetics | 0.0 | 0.0 |
| RNA 6005-N | | |
| Normal Bladder GENPAK 061001 | 4.2 | 4.0 |
| Bladder Cancer Research Genetics RNA | 3.7 | 0.7 |
| 1023 | | |
| Bladder Cancer INVITROGEN | 20.4 | 21.8 |
| A302173 | | |
| 87071 Bladder Cancer (OD04718-01) | 0.0 | 1.9 |
| 87072 Bladder Normal Adjacent | 1.4 | 0.7 |
| (OD04718-03) | | |
| Normal Ovary Res. Gen. | 1.7 | 4.4 |
| Ovarian Cancer GENPAK 064008 | 11.5 | 12.6 |
| 87492 Ovary Cancer (OD04768-07) | 0.0 | 0.0 |
| 87493 Ovary NAT (OD04768-08) | 1.3 | 0.0 |
| Normal Stomach GENPAK 061017 | 6.9 | 8.0 |
| Gastric Cancer Clontech 9060358 | 0.0 | 1.3 |
| NAT Stomach Clontech 9060359 | 5.3 | 5.4 |
| Gastric Cancer Clontech 9060395 | 1.2 | 0.7 |
| NAT Stomach Clontech 9060394 | 3.1 | 2.6 |
| Gastric Cancer Clontech 9060397 | 0.9 | 2.6 |
| NAT Stomach Clontech 9060396 | 2.2 | 0.7 |
| Gastric Cancer GENPAK 064005 | 2.2 | 4.4 |

TABLE 32
Panel 3D

| | Relative | Relative |
|---|---|---|
| | Expression | Expression |
| | (%) | (%) |
| | 3dx4tm6577f_{—} | 3dx4tm5098t_ |
| Tissue Name | Gpcr10_a1 | ag998_b2 |
| 94905_Daoy_Medulloblastoma/Cerebellum_sscDNA | 0.0 | 0.0 |
| 94906_TE671_Medulloblastom/Cerebellum_sscDNA | 0.3 | 0.0 |
| 94907_D283 Med_Medulloblastoma/Cerebellum_{—} | 1.6 | 0.1 |
| sscDNA | | |
| 94908_PFSK-1_Primitive | 0.2 | 0.0 |
| Neuroectodermal/Cerebellum_sscDNA | | |
| 94909_XF-498_CNS_sscDNA | 0.0 | 0.2 |
| 94910_SNB-78 CNS/glioma_sscDNA | 0.0 | 0.0 |
| 94911_SF-268_CNS/glioblastoma_sscDNA | 0.0 | 0.0 |
| 94912_T98G_Glioblastoma_sscDNA | 0.0 | 0.0 |
| 96776_SK-N-SH_Neuroblastoma (metastasis)_sscDNA | 16.4 | 8.6 |
| 94913_SF-295CNS/glioblastoma_sscDNA | 13.4 | 6.2 |
| 94914_Cerebellum_sscDNA | 5.5 | 2.8 |
| 96777_Cerebellum_sscDNA | 3.3 | 0.0 |
| 94916_NCI-H292_Mucoepidermoid lung carcinoma_{—} | 1.2 | 0.0 |
| sscDNA | | |
| 94917_DMS-114_Small cell lung cancer_sscDNA | 0.0 | 0.0 |
| 94918_DMS-79_Small cell lung | 0.3 | 0.0 |
| cancer/neuroendocrine_sscDNA | | |
| 94919_NCI-H146_Small cell lung | 100.0 | 100.0 |
| cancer/neuroendocrine_sscDNA | | |
| 94920_NCI-H526_Small cell lung | 1.9 | 0.6 |
| cancer/neuroendocrine_sscDNA | | |
| 94921_NCI-N417_Small cell lung | 11.7 | 5.1 |
| cancer/neuroendocrine_sscDNA | | |
| 94923_NCI-H82_Small cell lung | 0.0 | 0.2 |
| cancer/neuroendocrine_sscDNA | | |
| 94924_NCI-H157_Squamous cell lung cancer | 0.0 | 0.0 |
| (metastasis)_sscDNA | | |
| 94925_NCI-H1155_Large cell lung | 0.2 | 0.3 |
| cancer/neuroendocrine_sscDNA | | |
| 94926_NCI-H1299_Large cell lung | 0.0 | 0.0 |
| cancer/neuroendocrine_sscDNA | | |
| 94927_NCI-H727_Lung carcinoid_sscDNA | 1.0 | 1.1 |
| 94928_NCI-UMC-11_Lung carcmoid_sscDNA | 5.5 | 3.1 |
| 94929_LX-1_Small cell lung cancer_sscDNA | 0.0 | 0.0 |
| 94930_Colo-205_Colon cancer_sscDNA | 0.0 | 0.0 |
| 94931_KM12_Colon cancer_sscDNA | 0.0 | 0.0 |
| 94932_KM20L2_Colon cancer_sscDNA | 0.0 | 0.0 |
| 94933_NCI-H716_Colon cancer_sscDNA | 0.9 | 0.2 |
| 94935_SW-48_Colon adenocarcinoma_sscDNA | 0.0 | 0.0 |
| 94936_SW1116_Colon adenocarcinoma_sscDNA | 0.0 | 0.0 |
| 94937_LS 174T_Colon adenocarcinoma_sscDNA | 0.0 | 0.0 |
| 94938_SW-948_Colon adenocarcmoma_sscDNA | 0.0 | 0.0 |
| 94939_SW-480_Colon adenocarcinoma_sscDNA | 0.0 | 0.0 |
| 94940_NCI-SNU-5_Gastric carcinoma_sscDNA | 0.0 | 0.0 |
| 94941_KATO III_Gastric carcinoma_sscDNA | 0.0 | 0.0 |
| 94943_NCI-SNU-16_Gastric carcinoma_sscDNA | 0.0 | 0.0 |
| 94944_NCI-SNU-1_Gastric carcinoma_sscDNA | 0.0 | 0.0 |
| 94946_RF-1_Gastric adenocarcinoma_sscDNA | 0.0 | 0.0 |
| 94947_RF-48_Gastric adenocarcinoma_sscDNA | 0.0 | 0.0 |
| 96778_MKN-45_Gastric carcinoma_sscDNA | 0.0 | 0.0 |
| 94949_NCI-N87_Gastric carcinoma_sscDNA | 0.0 | 0.0 |
| 94951_OVCAR-5_Ovarian carcinoma_sscDNA | 0.0 | 0.0 |
| 94952_RL95-2_Uterine carcinoma_sscDNA 1 | 0.0 | 0.0 |
| 94953_HelaS3_Cervical adenocarcinoma_sscDNA | 1.8 | 0.0 |
| 94954_Ca Ski_Cervical epidermoid carcinoma | 0.0 | 0.0 |
| (metastasis)_sscDNA | | |
| 94955_ES-2_Ovarian clear cell carcinoma_sscDNA | 0.0 | 0.2 |
| 94957_Ramos/6h stim_″; Stimulated with PMA/ | 0.0 | 0.0 |
| ionomycin 6h_sscDNA | | |
| 94958_Ramos/14h stim_″; Stimulated with PMA/ | 0.0 | 0.0 |
| ionomycin 14h_sscDNA | | |
| 94962_MEG-01_Chronic myelogenous leukemia | 5.3 | 2.1 |
| (megokaryoblast)_sscDNA | | |
| 94963_Raji_Burkitt's lymphoma_sscDNA | 0.0 | 0.0 |
| 94964_Daudi_Burkitt's lymphoma_sscDNA | 0.0 | 0.0 |
| 94965_U266_B-cell plasmacytoma/myeloma_sscDNA | 0.0 | 0.0 |
| 94968_CA46_Burkitt's lymphoma_sscDNA | 0.0 | 0.0 |
| 94970_RL_non-Hodgkin's B-cell lymphoma_sscDNA | 0.0 | 0.0 |
| 94972_JM1_pre-B-cell lymphoma/leukemia_sscDNA | 0.0 | 0.0 |
| 94973_Jurkat_T cell leukemia_sscDNA | 0.0 | 0.0 |
| 94974_TF-1_Erythroleukemia_sscDNA | 0.0 | 0.0 |
| 94975_HUT 78_T-cell lymphoma_sscDNA | 1.1 | 0.0 |
| 94977_U937_Histiocytic lymphoma_sscDNA | 0.0 | 0.0 |
| 94980_KU-812_Myelogenous leukemia_sscDNA | 24.2 | 10.2 |
| 94981_769-P_Clear cell renal carcinoma_sscDNA | 0.0 | 0.0 |
| 94983_Caki-2_Clear cell renal carcinoma_sscDNA | 0.7 | 0.0 |
| 94984_SW 839_Clear cell renal carcinoma_sscDNA | 0.0 | 0.0 |
| 94986_G401_Wilms′ tumor_sscDNA | 0.0 | 0.0 |
| 94987_Hs766T_Pancreatic carcinoma (LN metastasis)_{—} | 0.4 | 0.0 |
| sscDNA | | |
| 94988_CAPAN-1_Pancreatic adenocarcinoma (liver | 0.0 | 0.0 |
| metastasis)_sscDNA | | |
| 94989_SU86.86_Pancreatic carcinoma (liver | 0.4 | 0.5 |
| metastasis)_sscDNA | | |
| 94990_BxPC-3_Pancreatic adenocarcinoma_sscDNA | 3.4 | 1.4 |
| 94991_HPAC_Pancreatic adenocarcinoma_sscDNA | 0.0 | 0.0 |
| 94992_MIA PaCa-2_Pancreatic carcinoma_sscDNA | 0.3 | 0.0 |
| 94993_CFPAC-1_Pancreatic ductal adenocarcinoma_{—} | 4.1 | 1.8 |
| sscDNA | | |
| 94994_PANC-1_Pancreatic epithelioid ductal | 0.0 | 0.0 |
| carcinoma_sscDNA | | |
| 94996_T24_Bladder carcinma (transitional cell)_sscDNA | 0.0 | 0.0 |
| 94997_5637_Bladder carcinoma_sscDNA | 4.4 | 1.5 |
| 94998_HT-1197_Bladder carcinoma_sscDNA | 6.4 | 6.0 |
| 94999_UM-UC-3_Bladder carcinma (transitional cell)_{—} | 0.8 | 0.0 |
| sscDNA | | |
| 95000_A204_Rhabdomyosarcoma_sscDNA | 0.0 | 0.0 |
| 95001_HT-1080_Fibrosarcoma_sscDNA | 0.0 | 0.0 |
| 95002_MG-63_Osteosarcoma (bone)_sscDNA | 0.0 | 0.0 |
| 95003_SK-LMS-1_Lelomyosarcoma (vulva)_sscDNA | 0.0 | 0.0 |
| 95004_SJRH30_Rhabdomyosarcoma (met to bone | 2.1 | 2.4 |
| marrow)_sscDNA | | |
| 95005_A431_Epideimoid carcinoma_sscDNA | 0.0 | 0.0 |
| 95007_WM2664_Melanoma_sscDNA | 7.2 | 4.3 |
| 95010_DU 145_Prostate carcinoma (brain metastasis)_{—} | 0.0 | 0.0 |
| sscDNA | | |
| 95012_MDA-MB-468_Breast adenocarcinoma_{—} | 0.0 | 0.3 |
| sscDNA | | |
| 95013_SCC-4_Squamous cell carcinoma of tongue_{—} | 0.0 | 0.0 |
| sscDNA | | |
| 95014_SCC-9_Squamous cell carcinoma of tongue_{—} | 0.0 | 0.0 |
| sscDNA | | |
| 95015_SCC-15_Squamous cell carcinoma of tongue_{—} | 0.0 | 0.0 |
| sscDNA | | |
| 95017_CAL 27_Squamous cell carcinoma of tongue_{—} | 0.3 | 0.0 |
| sscDNA | | |

TABLE 33
Panel 4D

| | Relative | Relative |
|---|---|---|
| | Epression | Expression |
| | 4dxtm5136f_{—} | 4Dtm3395t_{—} |
| Tissue Name | gper10_b2 | ag998 |
| 93768_Secondary Th1_anti-CD28/anti-CD3 | 0.0 | 0.0 |
| 93769_Secondary Th2_anti-CD28/anti-CD3 | 1.3 | 0.0 |
| 93770_Secondary Tr1_anti-CD28/anti-CD3 | 0.5 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 in IL-2 | 0.0 | 0.0 |
| 93566_primary Th2_resting dy 4-6 in IL-2 | 0.0 | 0.0 |
| 93567_primary Th2_resting dy 4-6 in IL-2 | 0.0 | 0.0 |
| 93351_CD45RA CD4 lymphocyte_anti-CD28/anti-CD3 | 0.0 | 0.3 |
| 93352_CD45RO CD4 lymphocyte_anti-CD28/anti-CD3 | 0.0 | 1.6 |
| 93251_CD8 Lymphocytes_anti-CD28/anti-CD3 | 1.4 | 0.0 |
| 93353_chronic CD8 Lymphocytes 2ry_resting dy 4-6 in IL-2 | 0.0 | 0.5 |
| 93574_chronic CD8 Lymphocytes 2ry_activated CD3/CD28 | 0.0 | 0.0 |
| 93354_CD4_none | 1.9 | 0.0 |
| 93252_Secondary Th1/Th2/Tr1_anti-CD95 CH11 | 0.0 | 0.0 |
| 93103_LAK cells_resting | 1.6 | 2.3 |
| 93788_LAX cells_IL-2 | 6.7 | 12.2 |
| 93787_LAX cells_IL-2 + IL-12 | 1.9 | 0.7 |
| 93789_LAX cells_IL-2 + IFN gamma | 2.9 | 4.6 |
| 93790_LAX cells_IL-2 + IL-18 | 2.6 | 4.4 |
| 93104_LAX cells_PMA/ionomycin and IL-18 | 3.2 | 0.6 |
| 93578_NK Cells IL-2_resting | 6.4 | 4.5 |
| 93109_Mixed Lymphocyte Reaction_Two Way MLR | 10.4 | 9.9 |
| 93110_Mixed Lymphocyte Reaction_Two Way MLR | 2.7 | 3.1 |
| 93111_Mixed Lymphocyte Reaction_Two Way MLR | 0.0 | 0.0 |
| 93112_Mononuclear Cells (PBMCs)_resting | 0.5 | 0.0 |
| 93113_Mononuclear Cells (PBMCs)_PWM | 3.2 | 1.3 |
| 93114_Mononuclear Cells (PBMCs)_PHA-L | 0.0 | 0.0 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphocytes_CD40L and IL-4 | 0.7 | 0.0 |
| 92665_EOL-1 (Eosinophil)_dbcAMP differentiated | 0.0 | 0.0 |
| 93248_EOL-1 (Eosinophil)_dbcAMP/PMAionomycln | 1.7 | 0.0 |
| 93356_Dendritic Cells_none | 0.8 | 0.9 |
| 93355_Dendritic Cells_LPS 100 ng/ml | 0.0 | 0.6 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.0 | 0.0 |
| 93098_HUVEC (Endothelial)_none | 0.0 | 0.0 |
| 93099_HUVEC (Endothelial)_starved | 1.0 | 0.0 |
| 93100_HUVEC (Endothelial)_IL-1b | 0.0 | 0.0 |
| 93779_HUVEC (Endothelial)_IFN gamma | 0.6 | 0.0 |
| 93102_HUVEC (Endothelial)_TNF alpha + IFN gamma | 0.0 | 0.0 |
| 93101_HUVEC (Endothelial)_TNF alpha + IL4 | 0.0 | 1.3 |
| 93781_HUVEC (Endothelial)_IL-11 | 1.3 | 0.0 |
| 93583_Lung Microvascular Endothelial Cells_none | 1.0 | 0.0 |
| 93584_Lung Microvascular Endothelial Cells_TNFa (4 ng/ml) | 1.9 | 0.0 |
| and IL1b (1 ng/ml) | | |
| 92662_Microvascular Dermal endothelium_none | 0.0 | 0.0 |
| 92663_Microsvasular Dermal endothelium_TNFa (4 ng/ml) and | 0.0 | 0.0 |
| IL1b (1 ng/ml) | | |
| 93773_Bronchial epithelium_TNFa (4 ng/ml) and IL1b (1 | 2.2 | 1.1 |
| ng/ml) ** | | |
| 93347_Small Airway Epithelium_none | 1.1 | 0.4 |
| 93348_Small Airway Epithelium_TNFa (4 ng/ml) and IL1b (1 | 0.0 | 0.0 |
| ng/ml) | | |
| 92668_Coronery Artery SMC_resting | 0.0 | 0.0 |
| 92669_Coronery Artery SMC_TNFa (4 ng/ml) and IL1b (1 | 0.0 | 0.0 |
| ng/ml) | | |
| 93107_astrocytes_resting | 0.0 | 0.3 |
| 93108_astrocytes_TNFa (4 ng/ml) and IL1b (1 ng/ml) | 0.9 | 0.7 |
| 92666_KU-812 (Basophil)_resting | 42.8 | 43.8 |
| 92667_KU-812 (Basophil)_PMA/ionoycn | 100.0 | 100.0 |
| 93579_CCD1106 (Keratinocytes)_none | 0.0 | 2.6 |
| 93580_CCD1106 (Keratinocytes)_TNFa and IFNg** | 0.0 | 0.6 |
| 93791_Liver Cirrhosis | 4.6 | 3.4 |
| 93792_Lupus Kidney | 0.0 | 0.0 |
| 93577_NCI-H292 | 1.9 | 0.0 |
| 93358_NCI-H292_IL-4 | 0.0 | 0.0 |
| 93360_NCI-H292_IL-9 | 0.9 | 0.2 |
| 93359_NCI-H292_IL-13 | 1.0 | 0.0 |
| 93357_NCI-H292_IFN gamma | 0.0 | 1.0 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast TNFa (4 ng/ml) and IL- | 0.0 | 0.3 |
| 1b (1 ng/ml) | | |
| 93257_Normal Human Lung Fibroblast_IL-4 | 0.0 | 0.0 |
| 93256_Normal Human Lung Fibroblast_IL-9 | 0.0 | 0.0 |
| 93255_Normal Human Lung Fibroblast_IL-13 | 0.0 | 0.0 |
| 93258_Normal Human Lung Fibroblast_IFN gamma | 0.0 | 0.3 |
| 93106_Dermal Fibroblasts CCD1070_resting | 3.6 | 0.0 |
| 93361_Dermal Fibroblasts CCD1070_TNF alpha 4 ng/ml | 0.0 | 0.0 |
| 93105_Dermal Fibroblasts CCD 1070_IL-1 beta 1 ng/ml | 0.0 | 0.7 |
| 93772_dermal fibroblast_IFN gamma | 0.0 | 0.0 |
| 93771_dermal fibroblast_IL-4 | 1.0 | 0.0 |
| 93259_IBD Colitis 1** | 1.3 | 0.0 |
| 93260_IBD Colitis 2 | 1.1 | 1.1 |
| 93261_IBD Crohns | 1.5 | 0.0 |
| 735010_Colon_normal | 0.0 | 0.0 |
| 735019_Lung_none | 0.0 | 0.7 |
| 64028-1_Thymus_none | 12.2 | 17.2 |
| 64030-1_Kidney_none | 4.8 | 5.0 |

TABLE 34
Panel CNSD.01

| | Relative | | Relative |
|---|---|---|---|
| | Expression (%) | | Expression (%) |
| | cns_1x4tm665 | | cns_1x4tm665 |
| Tissue Name | 1f_gpcr10 b1 | Tissue Name | 1f_gper10_b1 |
| 102633_BA4 Control | 39.1 | 102605_BA17 PSP | 36.8 |
| 102641_BA4 Control 2 | 27.9 | 102612_BA17 PSP2 | 16.2 |
| 102625_BA4 Alzheimer's 2 | 9.8 | 102637_Sub Nigra Control | 18.4 |
| 102649_BA4 Parkinson's | 55.7 | 102645 Sub Nigra Control 2 | 12.4 |
| 102656 BA4 Parkinson's2 | 71.6 | 102629_Sub Nigra | 12.6 |
| | | Alzheimer's 2 | |
| 102664_BA4 Huntington's | 40.3 | 102660_Sub Nigra | 40.0 |
| | | Parkinson's 2 | |
| 102671 BA4 Huntington's 2 | 10.7 | 102667_Sub Nigra | 34.5 |
| | | Huntington's | |
| 102603_BA4 PSP | 15.3 | 102674_Sub Nigra | 20.8 |
| | | Huntington's 2 | |
| 102610_BA4 PSP2 | 47.2 | 102614_Sub Nigra PSP2 | 2.6 |
| 102588_BA4 Depression | 19.3 | 102592_Sub Nigra Depression | 1.3 |
| 102596_BA4 Depression 2 | 10.0 | 102599_Sub Nigra Depression | 7.9 |
| | | 2 | |
| 102634_BA7 Control 1 | 49.7 | 102636_Glob Palladus Control | 3.7 |
| 102642_BA7 Control 2 | 27.2 | 102644_Glob Palladus Control | 9.7 |
| | | 2 | |
| 102626 BA7 Alzheimer's 2 | 19.1 | 1102620_Glob Palladus | 9.9 |
| | | Alzheimer's | |
| 102650_BA7 Parkinson's | 22.5 | 102628_Glob Palladus | 0.0 |
| | | Alzheimer's 2 | |
| 102657_BA7 Parkmson's 2 | 66.8 | 102652_Glob Palladus | 30.3 |
| | | Parkinson's | |
| 102665_BA7 Huntington's | 48.2 | 102659_Glob Palladus | 1.4 |
| | | Parkinson's 2 | |
| 102672_BA7 Huntington's 2 | 53.4 | 102606_Glob Palladus PSP 0.0 | |
| 102604_BA7 PSP | 49.6 | 102613_Glob Palladus PSP2 | 1.5 |
| 102611_BA7 PSP 2 | 39.3 | 102591_Glob Palladus | 0.0 |
| | | Depression | |
| 102589_BA7 Depression | 18.1 | 102638_Temp Pole Control | 25.2 |
| 102632_BA9 Control | 37.7 | 102646_Temp Pole Control 2 | 81.6 |
| 102640_BA9 Control 2 | 69.3 | 102622_Temp Pole Alzheimer's | 12.7 |
| 102617_BA9 Alzheimer's | 8.9 | 102630_Temp Pole | 17.2 |
| | | Alzheimer's 2 | |
| 102624_BA9 Alzheimer's 2 | 26.4 | 102653_Temp Pole Parkinson's | 46.6 |
| 102648_BA9 Parkinson's | 29.4 | 102661_Temp Pole | 40.7 |
| | | Parkinson's 2 | |
| 102655_BA9 Parkinson's 2 | 55.8 | 102668_Temp Pole | 66.3 |
| | | Huntington's | |
| 102663_BA9 Huntington's | 51.3 | 102607 Temp Pole PSP | 5.7 |
| 102670_BA9 Huntington's 2 | 21.1 | 102615 Temp Pole PSP2 | 12.4 |
| 102602_BA9 PSP | 27.6 | 102600_Temp Pole | 9.6 |
| | | Depression 2 | |
| 102609_BA9 PSP 2 | 13.1 | 102639_Cing Gyr Control | 57.2 |
| 102587_BA9 Depression | 13.8 | 102647_Cing Gyr Control 2 | 27.5 |
| 102595_BA9 Depression 2 | 7.2 | 102623_Cing Gyr Alzheimer's | 25.1 |
| 102635_BA17 Control | 100.0 | 102631_Cing Gyr Alzheimer's | 6.8 |
| | | 2 | |
| 102643_BA17 Control 2 | 53.3 | 102654_Cing Gyr Parkinson's | 24.8 |
| 102627_BA17 Alzheimer's 2 | 19.6 | 102662_Cing Gyr Parkinson's 2 | 36.7 |
| 102651_BA17 Parkinson's | 67.7 | 102669_Cing Gyr Huntington's | 60.3 |
| 102658_BA17 Parkinson's 2 | 77.0 | 102676_Cing Gyr | 16.4 |
| | | Huntington's 2 | |
| 102666_BA17 Huntington's | 43.9 | 102608_Cing Gyr PSP | 19.0 |
| 102673_BA17 Huntington's 2 | 23.5 | 102616_Cing Gyr PSP 2 | 6.9 |
| 102590_BA17 Depression | 16.9 | 102594_Cing Gyr Depression | 9.3 |
| 102597_BA17 Depression 2 | 33.1 | 102601_Cing Gyr Depression 2 | 15.4 |

Panel 1 Summary:

Gpcr10 The NOV4 gene is relatively highly expressed in samples from the central nervous system. Among these tissues, moderate expression is detected in thalamus, hippocampus, amygdala and substantia nigra, while lower expression is seen in spinal cord, hypothalamus and cerebellum (see discussion of Panel 1.3D for potential utility). Among normal tissues, NOV4 gene expression is also detected in colon, kidney, thyroid, testis and uterus

The NOV4 gene is most highly expressed in a sample derived from a lung cancer cell line and shows significant expression in other samples derived from lung cancer cell lines. In addition, there appears to be significant expression of this gene in CNS cancer derived cell lines, ovarian cancer cell lines, and a pancreatic cancer cell line. Thus, based upon this pattern of gene expression, the therapeutic modulation of the activity of the NOV4 gene product is of use in the treatment of CNS malignancies, lung cancer, pancreatic cancer and/or ovarian cancer.

Panel 1.1 Summary:

Gpcr10/Gpcr38 Three replicate experiments performed using different probe/primer sets yielded results that are in good agreement. Strong expression of the NOV4 gene is again observed in the CNS, including in amygdala, cerebellum, hippocampus, substantia nigra, thalamus and cerebral cortex. Lower expression levels are also seen in the spinal cord. This gene shows homology to Slit-3, and shows brain preferential expression. The Slits are a family of secreted guidance proteins that can repel neuronal migration and axon growth via interaction with their cellular roundabout receptors, making this an excellent candidate neuronal guidance protein for axons, dendrites and/or growth cones in general (Ref. 2-3). Therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Among metabolically relevant tissues, NOV4 gene expression is seen in fetal skeletal muscle, pancreas, and pituitary gland. This observation suggests that therapeutic modulation may aid the treatment of metabolic diseases such as obesity and diabetes as well as neuroendocrine disorders. Glycoprotein hormones influence the development and function of the ovary, testis and thyroid by binding to specific high-affinity receptors. Interestingly, the extracellular domains of these receptors are members of the leucine-rich repeat (LRR) protein superfamily and are responsible for the high-affinity binding (Ref. 1).

Similar to what was observed in Panel 1, the NOV4 gene shows highest expression in a sample derived from a lung cancer cell line and also shows significant over-expression in other samples derived from lung cancer cell lines relative to the normal lung control. Furthermore, it is also highly expressed by brain tumors derived cell lines, indicating a possible role in the development and progression of brain tumors. There appears to be significant expression of the NOV4 gene in a melanoma cell line as well as in uterus and testis tissue. Thus, based upon this pattern of gene expression, the therapeutic modulation of the activity of the NOV4 gene product is of use in the treatment of CNS malignancies, melanomas and/or lung cancer.

Panel 1.2 Summary:

Gpcr10 Expression of the NOV4 gene is low/undetectable (CT values>35) in all samples on this panel (data not shown).

Panel 1.3D Summary:

Gpcr10/Ag998 Results from two replicate experiments were performed using different probe/primer sets and the results are in excellent agreement. The NOV4 gene is most highly expressed in cerebral cortex (CT=30) and shows moderate expression in other CNS regions as well including, amygdala, hippocampus, and thalamus. The NOV4 gene encodes a leucine-rich repeat protein. Leucine rich repeats (LRR) mediate reversible protein-protein interactions and have diverse cellular functions, including cellular adhesion and signaling. Several of these proteins, such as connectin, slit, chaoptin, and Toll have pivotal roles in neuronal development in Drosophila and may play significant but distinct roles in neural development and in the adult nervous system of humans (Ref. 2). In Drosophilia, the LRR region of axon guidance proteins has been shown to be critical for their function (especially in axon repulsion). Since the leucine-rich-repeat protein encoded by the NOV4 gene shows high expression in the cerebral cortex, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Among normal tissues, expression of the NOV4 gene is also seen in thyroid (CT=34), fetal skeletal muscle (CT=33), uterus (CT=32) and testis (CT=33). In addition, there is a strong cluster of expression in CNS cancer-derived cell lines and lung cancer cell lines. Thus, based upon this pattern of gene expression, the therapeutic modulation of the activity of the NOV4 gene product is of use in the treatment of CNS malignancies or lung cancer.

Panel 2D Summary:

Gpcr10/Ag998 Results from two replicate experiments were performed using different probe/primer sets and the results are in excellent agreement. The NOV4 gene is most highly expressed in a sample derived from a melanoma metastasis (CT=30.9). In addition, this gene appears to be more highly expressed in normal kidney and thyroid tissues when compared to associated cancer tissues. In contrast, the NOV4 gene is more highly expressed in lung cancer tissue when compared to normal adjacent tissue. Thus, therapeutic up-regulation of the activity of this gene, through the application of the protein product itself or by gene replacement therapy, is of use in the treatment of kidney and thyroid cancer. Alternatively, down-regulation of the activity of the NOV4 gene product, through the use of inhibitory antibodies or small molecule drugs, is of use in the treatment of melanoma or lung cancer.

Panel 3D Summary:

Gpcr10/Ag998 Results from two replicate experiments were performed using different probe/primer sets and the results are in excellent agreement. The highest expression of the NOV4 gene on this panel is detected in a cell line derived from a small cell lung cancer (CT=29.1). In addition, there is expression in a cluster of lung cancer cell lines indicating that the inhibition of this gene activity is of use in the therapy of lung cancer. This result is consistent with what was observed in Panel 1.3D and Panel 2D.

Panel 4D Summary:

Gpcr10/Ag998 Results from two replicate experiments were performed using different probe/primer sets and the results are in excellent agreement. The NOV4 transcript is induced in PMA and ionomycin treated basophil cell line KU-812. Basophils release histamines and other biological modifiers in repose to allergens and play an important role in the pathology of asthma and hypersensitivity reactions. Therefore, antibody therapeutics designed against the putative leucine rich repeat protein encoded for by the NOV4 gene could reduce or inhibit inflammation by blocking basophil function in these diseases.

Panel CNSD.01 Summary:

Gpcr10 The NOV4 gene shows highest expression throughout the cortex, with lower levels in the substantia nigra and globus palladus. This result is consistent with what was observed in Panels 1, 1.1, and 1.3D. In addition, there is no apparent association between the NOV4 gene expression pattern and the diseased samples present on this panel.

NOV5

Expression of gene NOV5 was assessed using the primer-probe set Ag1439, described in Table 35. Results from RTQ-PCR runs are shown in Tables 36, 37, and 38.

TABLE 35
Probe Name Ag1439

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-TCTCTTAGCCGTCATTGTCAGT-3′ | 59 | 22 | 2508 | 93 |
| Probe | FAM-5′- | 69.3 | 26 | 2553 | 94 |
| | TAGAATCAGCCTCAAGAGCTGGCACA- | | | | |
| | 3′-TAMRA | | | | |
| Reverse | 5′-GAAAGCACAAGTTCACAAGCA-3′ | 59.1 | 21 | 2579 | 95 |

TABLE 36
Panel 1.2

| | Relative | | Relative |
|---|---|---|---|
| | Expression (%) | | Expression (%) |
| | 1.2tm1799f_{—} | | 1.2tm1799f_{—} |
| Tissue Name | ag1439 | Tissue Name | ag1439 |
| Endothelial cells | 12.9 | Renal ca. 786-0 | 7.4 |
| Heart (fetal) | 39.2 | Renal Ca. A498 | 7.3 |
| Pancreas | 1.6 | Renal Ca. RXF 393 | 4.0 |
| Pancreatic ca. CAPAN 2 | 10.7 | Renal Ca. ACHN | 9.4 |
| Adrenal Gland (new lot*) | 14.7 | Renal ca. UO-31 | 19.6 |
| Thyroid | 4.4 | Renal Ca. TK-10 | 15.4 |
| Salivary gland | 12.0 | Liver | 53.6 |
| Pituitary gland | 1.0 | Liver (fetal) | 2.9 |
| Brain (fetal) | 0.9 | Liver ca. (hepatoblast) HepG2 | 57.0 |
| Brain (whole) | 4.6 | Lung | 0.2 |
| Brain (amygdala) | 7.0 | Lung (fetal) | 1.1 |
| Brain (cerebellum) | 1.5 | Lung ca. (small cell) LX-1 | 14.6 |
| Brain (hippocampus) | 16.8 | Lung ca. (small cell) NCI-H69 | 6.7 |
| Brain (thalamus) | 9.7 | Lung ca. (s.cell var.) SHP-77 | 1.7 |
| Cerebral Cortex | 23.3 | Lung ca. (large cell)NCI-H460 | 25.0 |
| Spinal cord | 1.3 | Lung ca. (non-sm. cell) A549 | 10.4 |
| CNS Ca. (glio/astro) U87-MG | 11.3 | Lung ca. (non-s.cell) NCI-H23 | 50.3 |
| CNS ca. (glio/astro) U-118-MG | 9.2 | Lung ca (non-s.cell) HOP-62 | 36.9 |
| CNS ca. (astro) SW1783 | 3.5 | Lung ca. (non-s.cl) NCI-H522 | 76.3 |
| CNS ca.* (neuro; met) SK-N- | 23.8 | Lung Ca. (squam.) SW 900 | 57.4 |
| AS | | | |
| CNS ca. (astro) SF-539 | 2.4 | Lung ca. (squam.) NCI-H596 | 16.2 |
| CNS Ca. (astro) SNB-75 | 3.1 | Mammary gland | 1.3 |
| CNS ca. (glio) SNB-19 | 23.0 | Breast ca.* (pl. effusion) MCF-7 | 4.6 |
| CNS ca. (glio) U251 | 7.0 | Breast ca.* (pl.ef) MDA-MB-231 | 3.3 |
| CNS ca. (glio) SF-295 | 32.1 | Breast ca.* (pl. effusion) T47D | 5.0 |
| Heart | 55.1 | Breast ca. BT-549 | 3.4 |
| Skeletal Muscle (new lot*) | 100.0 | Breast ca. MDA-N | 26.6 |
| Bone marrow | 0.9 | Ovary | 7.6 |
| Thymus | 0.3 | Ovarian ca. OVCAR-3 | 27.5 |
| Spleen | 0.7 | Ovarian ca. OVCAR-4 | 12.1 |
| Lymph node | 0.0 | Ovarian ca. OVCAR-5 | 54.3 |
| Colorectal | 3.3 | Ovarian ca. OVCAR-8 | 7.9 |
| Stomach | 1.8 | Ovarian ca. IGROV-1 | 12.6 |
| Small intestine | 10.0 | Ovarian ca.* (ascites) SK-OV-3 | 47.0 |
| Colon ca. SW480 | 3.6 | Uterus | 4.5 |
| Colon ca.* (SW480 met)SW620 | 15.8 | Placenta | 1.4 |
| Colon Ca. HT29 | 6.6 | Prostate | 11.5 |
| Colon Ca. HCT-116 | 34.4 | Prostate ca.* (bone met)PC-3 | 26.2 |
| Colon Ca. CaCo-2 | 15.4 | Testis | 1.1 |
| 83219 CC Well to Mod Diff | 0.7 | Melanoma Hs688(A).T | 3.2 |
| (ODO3866) | | | |
| Colon Ca. HCC-2998 | 46.3 | Melanoma* (met) Hs688(B).T | 1.9 |
| Gastric ca.* (liver met) NCI- | 20.2 | Melanoma UACC-62 | 12.5 |
| N87 | | | |
| Bladder | 17.6 | Melanoma M14 | 13.5 |
| Trachea | 0.7 | Melanoma LOX IMVI | 3.0 |
| Kidney | 55.1 | Melanoma* (met) SK-MEL-5 | 20.2 |
| Kidney (fetal) | 5.4 | Adipose | 3.5 |

TABLE 37
Panel 2D

| | Relative Expression (%) | |
|---|---|---|
| | 2Dtm2334f_{—} | 2Dtm2365f_{—} |
| Tissue Name | ag1439 | ag1439 |
| Normal Colon GENPAK 061003 | 53.2 | 50.7 |
| 83219 CC Well to Mod Diff(ODO3866) | 3.5 | 3.4 |
| 83220 CC NAT (ODO3866) | 15.3 | 13.6 |
| 83221 CC Gr.2 rectosigmoid (ODO3868) | 7.4 | 7.0 |
| 83222 CC NAT (ODO3868) | 4.4 | 5.0 |
| 83235 CC Mod Diff (ODO3920) | 8.9 | 7.4 |
| 83236 CC NAT (ODO3920) | 16.0 | 14.5 |
| 83237 CC Gr.2 ascend colon (ODO3921) | 24.0 | 23.2 |
| 83238 CC NAT (ODO3921) | 6.8 | 9.8 |
| 83241 CC from Partial Hepatectomy | 13.1 | 11.8 |
| (ODO4309) | | |
| 83242 Liver NAT (ODO4309) | 54.7 | 50.0 |
| 87472 Colon mets to lung (ODO4451-01) | 12.9 | 7.6 |
| 87473 Lung NAT (ODO4451-02) | 2.7 | 3.3 |
| Normal Prostate Clontech A + 6546-1 | 13.7 | 26.6 |
| 84140 Prostate Cancer (ODO4410) | 20.6 | 26.1 |
| 84141 Prostate NAT (ODO4410) | 17.2 | 17.9 |
| 87073 Prostate Cancer (ODO4720-01) | 14.1 | 14.0 |
| 87074 Prostate NAT (ODO4720-02) | 29.5 | 28.3 |
| Normal Lung GENPAK 061010 | 7.0 | 7.1 |
| 83239 Lung Met to Muscle (ODO4286) | 6.5 | 8.3 |
| 83240 Muscle NAT (ODO4286) | 13.8 | 15.2 |
| 84136 Lung Malignant Cancer (ODO3126) | 10.1 | 9.9 |
| 84137 LungNAT (ODO3126) | 6.5 | 9.1 |
| 84871 Lung Cancer (ODO4404) | 5.4 | 6.3 |
| 84872 Lung NAT (ODO4404) | 9.0 | 12.3 |
| 84875 Lung Cancer (ODO4565) | 5.0 | 3.3 |
| 84876 Lung NAT (ODO4565) | 1.3 | 1.7 |
| 85950 Lung Cancer (ODO4237-01) | 33.7 | 43.5 |
| 85970 Lung NAT (ODO4237-02) | 6.7 | 8.5 |
| 83255 Ocular Mel Met to Liver (ODO4310) | 17.8 | 14.2 |
| 83256 Liver NAT (ODO4310) | 70.2 | 63.3 |
| 84139 Melanoma Mets to Lung (ODO4321) | 11.7 | 13.9 |
| 84138 LungNAT (ODO4321) | 9.2 | 9.5 |
| Normal Kidney GENPAK 061008 | 35.8 | 41.2 |
| 83786 Kidney Ca, Nuclear grade 2 | 25.5 | 27.2 |
| (ODO4338) | | |
| 83787 Kidney NAT (ODO4338) | 10.6 | 9.8 |
| 83788 Kidney Ca Nuclear grade 1/2 | 16.5 | 21.8 |
| (ODO4339) | | |
| 83789 Kidney NAT (ODO4339) | 21.0 | 20.9 |
| 83790 Kidney Ca, Clear cell type | 13.2 | 12.6 |
| (ODO4340) | | |
| 83791 Kidney NAT (ODO4340) | 16.8 | 16.4 |
| 83792 Kidney Ca, Nuclear grade 3 | 2.1 | 3.2 |
| (ODO4348) | | |
| 83793 Kidney NAT (ODO4348) | 7.3 | 7.1 |
| 87474 Kidney Cancer (ODO4622-01) | 5.1 | 7.3 |
| 87475 Kidney NAT (ODO4622-03) | 2.7 | 2.9 |
| 85973 Kidney Cancer (ODO4450-01) | 33.7 | 33.9 |
| 85974 Kidney NAT (ODO4450-03) | 26.1 | 14.4 |
| Kidney Cancer Clontech 8120607 | 3.6 | 3.8 |
| Kidney NAT Clontech 8120608 | 13.9 | 8.3 |
| Kidney Cancer Clontech 8120613 | 4.6 | 5.0 |
| Kidney NAT Clontech 8120614 | 6.9 | 5.8 |
| Kidney Cancer Clontech 9010320 | 15.0 | 14.3 |
| KidneyNAT Clontech 9010321 | 12.9 | 14.9 |
| Normal Uterus GENPAK 061018 | 5.4 | 6.9 |
| Uterus Cancer GENPAK 064011 | 23.0 | 22.2 |
| Normal Thyroid Clontech A + 6570-1 | 46.3 | 68.8 |
| Thyroid Cancer GENPAK 064010 | 10.4 | 14.2 |
| Thyroid Cancer INVITROGEN A302152 | 6.4 | 5.2 |
| Thyroid NAT INVITROGEN A302153 | 47.3 | 50.7 |
| Normal Breast GENPAK 061019 | 28.5 | 23.5 |
| 84877 Breast Cancer (ODO4566) | 2.5 | 1.5 |
| 85975 Breast Cancer (ODO4590-01) | 12.8 | 11.3 |
| 85976 Breast Cancer Mets (ODO4590-03) | 20.9 | 18.7 |
| 87070 Breast Cancer Metastasis | 25.0 | 24.3 |
| (ODO4655-05) | | |
| GENPAK Breast Cancer 064006 | 3.3 | 4.3 |
| Breast Cancer Res. Gen. 1024 | 4.1 | 22.2 |
| Breast Cancer Clontech 9100266 | 7.8 | 8.1 |
| Breast NAT Clontech 9100265 | 7.7 | 7.1 |
| Breast Cancer INVITROGEN A209073 | 26.1 | 25.3 |
| Breast NAT INVITROGEN A2090734 | 21.5 | 24.7 |
| Normal Liver GENPAK 061009 | 56.3 | 55.5 |
| Liver Cancer GENPAK 064003 | 100.0 | 100.0 |
| Liver Cancer Research Genetics RNA 1025 | 21.5 | 23.8 |
| Liver Cancer Research Genetics RNA 1026 | 5.4 | 4.3 |
| Paired Liver Cancer Tissue Research | 66.4 | 41.8 |
| Genetics RNA 6004-T | | |
| Paired Liver Tissue Research Genetics | 4.0 | 4.8 |
| RNA 6004-N | | |
| Paired Liver Cancer Tissue Research | 6.8 | 8.5 |
| Genetics RNA 6005-T | | |
| Paired Liver Tissue Research Genetics | 13.5 | 14.7 |
| RNA 6005-N | | |
| Normal Bladder GENPAK 061001 | 14.3 | 15.3 |
| Bladder Cancer Research Genetics | 3.3 | 3.3 |
| RNA 1023 | | |
| Bladder Cancer INVITROGEN A302173 | 12.6 | 12.5 |
| 87071 Bladder Cancer (ODO4718-01) | 4.7 | 5.6 |
| 87072 Bladder Normal Adjacent | 11.0 | 11.4 |
| (ODO4718-03) | | |
| Normal Ovary Res. Gen. | 6.0 | 3.8 |
| Ovarian Cancer GENPAK 064008 | 27.7 | 21.6 |
| 87492 Ovary Cancer (ODO4768-07) | 29.7 | 30.1 |
| 87493 Ovary NAT (ODO4768-08) | 7.0 | 7.2 |
| Nonnal Stomach GENPAK 061017 | 8.1 | 10.4 |
| Gastric Cancer Clontech 9060358 | 3.0 | 3.3 |
| NAT Stomach Clontech 9060359 | 6.2 | 4.6 |
| Gastric Cancer Clontech 9060395 | 7.6 | 7.7 |
| NAT Stomach Clontech 9060394 | 4.1 | 3.2 |
| Gastric Cancer Clontech 9060397 | 13.2 | 12.4 |
| NAT Stomach Clontech 9060396 | 2.7 | 1.6 |
| Gastric Cancer GENPAK 064005 | 5.9 | 5.8 |

TABLE 38
Panel 4D

| | Relative | | Relative |
|---|---|---|---|
| | Expression | | Expression |
| | (%) | | (%) |
| | 4dtm2199f_{—} | | 4dtm2199f_{—} |
| Tissue Name | ag1439 | Tissue Name | ag1439 |
| 93768_Secondary Th1_anti- | 17.8 | 93100_HUVEC | 13.1 |
| CD28/anti-CD3 | | (Endothelial)_IL-1b | |
| 93769_Secondary Th2_anti- | 13.7 | 93779_HUVEC | 27.4 |
| CD28/anti-CD3 | | (Endothelial) lEN gamma | |
| | | 93102_HUVEC | |
| 93770_Secondary Tr1_anti- | 14.8 | (Endothelial)_TNF alpha + IFN | 7.2 |
| GD28/anti-CD3 | | gamma | |
| 93573_Secondary Th1_resting | 0.5 | 93101_HUVEC | 26.8 |
| day 4-6 in IL-2 | | (Endothelial)_TNF alpha + IL4 | |
| 93572_Secondary Th2_resting | 0.8 | 93781_HUVEC | 11.6 |
| day 4-6 in IL-2 | | (Endothelial)_IL-11 | |
| 93571_Secondary Tr1_resting | 0.2 | 93583_Lung Microvascular | 15.4 |
| day 4-6 in IL-2 | | Endothelial Cells_none | |
| | | 93584_Lung Microvascular | |
| 93568_primary Th1_anti- | 58.2 | Endothelial Cells_TNFa (4 | 11.5 |
| CD28/anti-CD3 | | ng/ml) and IL1b (1 ng/ml) | |
| 93569_primary Th2_anti- | 56.6 | 92662_Microvascular Dermal | 22.4 |
| CD28/anti-CD3 | | endothelium_none | |
| | | 92663_Microsvasular Dermal | |
| 93570_primary Tr1_anti- | 74.7 | endothelium_TNFa (4 ng/ml) | 12.7 |
| CD28/anti-CD3 | | and IL1b (1 ng/ml) | |
| | | 93773_Bronchial | |
| 93565_primary Th1_resting dy | 3.0 | epithelium_TNFa (4 ng/ml) and | 84.1 |
| 4-6 in IL-2 | | IL1b (1 ng/ml)** | |
| 93566_primary Th2_resting dy | 2.5 | 93347_Small Airway | 31.2 |
| 4-6 in IL-2 | | Epithelium_none | |
| | | 93348_Small Airway | |
| 93567_primary Tr1_resting dy | 3.7 | Epithelium_TNFa (4 ng/ml) | 100.0 |
| 4-6 in IL-2 | | and IL1b (1 ng/ml) | |
| 93351_CD45RACD4 | 20.4 | | |
| lymphocyte_anti-CD28/anti- | | 92668_Coronery Artery | 13.2 |
| CD3 | | SMC_resting | |
| 93352_CD4SRO CD4 | 11.7 | 92669_Coronery Artery | 13.6 |
| lymphocyte_anti-CD2 8/anti- | | SMC_TNFa (4 ng/ml) and IL1b | |
| CD3 | | (1 ng/ml) | |
| 93251_CD8 Lymphocytes_anti- | 2.6 | | |
| CD28/anti-CD3 | | 93107_astrocytes_resting | 14.6 |
| 93353_chronic CD8 | 5.3 | | |
| Lymphocytes 2ry_resting dy 4- | | 93108_astrocytesTNFa (4 | 12.9 |
| 6 in IL-2 | | ng/ml) and IL1b (1 ng/ml) | |
| 93574_chronic CD8 | 3.5 | | |
| Lymphocytes 2ry_activated | | 92666_KU-8 12 | 5.7 |
| CD3/CD28 | | (Basophil)_resting | |
| | | 92667_KU-812 | 4.5 |
| 93354_CD4_none | 1.8 | (Basophil)_PMA/ionoycin | |
| 93252_Secondary | | 93579_CCD11O6 | |
| Th1/Th2/Trl_anti-CD95 CH11 | 2.0 | (Keratinocytes)_none | 36.6 |
| | | 93580_CCD1106 | 84.1 |
| | | (Keratinocytes)_TNFa and | |
| 93103_LAK cells_resting | 0.7 | IFNg** | |
| 93788_LAK cells_IL-2 | 1.3 | 93791_Liver Cirrhosis | 4.7 |
| 93787_LAK cells_IL-2 + IL-12 | 35.4 | 93792_Lupus Kidney | 6.7 |
| 93789_LAK cells_IL-2 + IFN | 5.8 | | |
| gamma | | 93577_NCI-H292 | 54.7 |
| 93790_LAK cells_IL-2 + IL-18 | 2.5 | 93358_NCI-H292_IL-4 | 59.5 |
| 93104_LAK | 1.2 | | |
| cells_PMA/ionomycin and IL- | | | |
| 18 | | 93360_NCI-H292_IL-9 | 68.3 |
| 93578_NK Cells IL-2_resting | 1.5 | 93359_NCI-H292_IL-13 | 48.3 |
| 93109_Mixed Lymphocyte | 0.7 | | |
| Reaction_Two Way MLR | | 93357_NCI-H292_IFN gamma | 13.9 |
| 93110_Mixed Lymphocyte | 3.3 | | |
| Reaction_Two Way MLR | | 93777_HPAEC_- | 15.1 |
| 93111_Mixed Lymphocyte | 3.5 | 93778_HPAEC_IL-1 beta/TNA | 15.7 |
| Reaction_Two Way MLR | | alpha | |
| 93112_Mononuclear Cells | 0.2 | 93254_Normal Human Lung | 12.2 |
| (PBMCs)_resting | | Fibroblast_none | |
| | | 93253_Normal Human Lung | 20.7 |
| 93113_Mononuclear Cells | 19.1 | Fibroblast_TNFa (4 ng/ml) and | |
| (PBMCs)_PWM | | IL-lb (1 ng/ml) | |
| 93114_Mononuclear Cells | 22.5 | 93257_Normal Human Lung | 28.5 |
| (PBMCs)_PHA-L | | Fibroblast_IL-4 | |
| | | 93256_Normal Human Lung | 25.2 |
| 93249_Ramos (B cell)_none | 0.3 | Fibroblast_IL-9 | |
| 93250_Ramos (B | 0.3 | 93255_Normal Human Lung | 46.7 |
| cell)_ionomycin | | Fibroblast_IL-13 | |
| 93258_Normal Human Lung | | | |
| 93349_B lymphocytes_PWM | 49.3 | Fibroblast_IEN gamma | 19.2 |
| 93350_B lymphoytes_CD40L | | 93106_Dermal Fibroblasts | |
| and IL-4 | 1.8 | CCD1070_resting | 40.1 |
| 92665_EOL-1 | 12.7 | | |
| (Eosinophil)_dbcAMP | | 93361_Dermal Fibroblasts | 44.4 |
| differentiated | | CCD1070 TNF alpha 4 ng/ml | |
| 93248_EOL-1 | 2.3 | | |
| (Eosinophil)_dbcAMP/PMAion | | 93105_Dermal Fibroblasts | 61.1 |
| omycin | | CCD1070_IL-1 beta 1 ng/ml | |
| | | 93772_dermal fibroblast_IFN | 2.9 |
| 93356_Dendritic Cells_none | 0.4 | gamma | |
| 93355_Dendritic Cells_LPS | 0.4 | | |
| 100 ng/ml | | 93771_dermal fibroblast_IL-4 | 12.7 |
| 93775_Dendritic Cells_anti- | 0.4 | | |
| CD40 | | 93259_IBD Colitis 1** | 9.2 |
| 93774_Monocytes_resting | 0.6 | 93260_IBD Colitis 2 | 1.4 |
| 93776_Monocytes_LPS 50 | 0.1 | | |
| ng/ml | | 93261_IBD Crohns | 3.8 |
| 93581_Macrophages resting | 1.8 | 735010_Colon_normal | 12.9 |
| 93582_Macrophages_LPS 100 | 0.3 | | |
| ng/ml | | 735019_Lung_none | 11.0 |
| 93098_HUVEC | 26.1 | | |
| (Endothelial)_none | | 64028-1_Thymus_none | 81.2 |
| 93099_HUVEC | 51.0 | 64030-1_Kidney_none | 7.7 |
| (Endothelial)_starved | | | |

Panel 1.2 Summary:

Ag1439 Expression of the NOV5 gene is highest in skeletal muscle (CT=24.2). However, the expression of this gene is quite widespread. Interestingly, NOV5 gene expression is preferentially seen in cancer cell lines compared to normal tissues, and in particular, notably higher gene expression is detected in ovarian cancer and lung cancer cell lines. Since normal cultured cell lines are highly proliferative, this observation may indicate that the expression of the NOV5 gene is used to distinguish proliferating cells over resting or quiescent cells. In addition, therapeutic modulation of the activity of this gene product is of use in the treatment of ovarian and lung cancer.

Among CNS tissues, high expression of this gene is detected in cerebral cortex (CT=26.3) and hippocampus (CT=26.8). More moderate expression is also detected in amygdala, cerebellum, thalamus and spinal cord. In Drosophilia, the LRR region of axon guidance proteins has been shown to be critical for function (especially in axon repulsion). The NOV5 gene encodes a protein with predicted leucine-rich-repeats, making it an excellent candidate neuronal guidance protein for axons, dendrites and/or growth cones in general. Therefore, therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). This protein also contains homology to the GPCR family of receptors. Several neurotransmitter receptors are GPCRs, including the dopamine receptor family, the serotonin receptor family, the GABAB receptor, muscarinic acetylcholine receptors, and others; thus this GPCR may represent a novel neurotransmitter receptor. Targeting various neurotransmitter receptors (dopamine, serotonin) has proven to be an effective therapy in psychiatric illnesses such as schizophrenia, bipolar disorder and depression. Furthermore the cerebral cortex and hippocampus are regions of the brain that are known to play critical roles in Alzheimer's disease, seizure disorders, and in the normal process of memory formation. Therapeutic modulation of this gene or its protein product may be beneficial in one or more of these diseases, as may stimulation and/or blockade of the receptor coded for by the gene. Levels of this gene are high, however, in areas outside of the central nervous system (such as the heart, muscle, liver and kidney), suggesting the possibility of a wider role in intercellular signaling.

Among metabolically relevant tissues, the NOV5 gene is expressed in heart and fetal heart (CT=25), pancreas (CT=30), adrenal gland (CT=27), thyroid (CT=29), pituitary gland (CT=31), skeletal muscle (CT=24), liver (CT=25) and fetal liver (CT=29). Therefore, this gene product may be a small-molecule target for the treatment of disease in metabolic tissues, such as diabetes and obesity.

Panel 2D Summary:

AR1439 Results from two replicate experiments using the same probe/primer set are in excellent agreement. Expression of the NOV5 gene in Panel 2D is highest in a sample derived from a liver cancer (CT=29.3). However, the gene is also expressed at more moderate levels in most of the other samples on this panel. In some instances there appears to be substantial dysregulation of expression with disease association. For example, overexpression of the NOV5 gene appears to be associated with ovarian, liver and gastric cancers. Thus, the modulation of the expression of this gene, or the function of its product, is of utility in the treatment of these cancers.

Panel 4D Summary:

Ag1439 The NOV5 gene is expressed in numerous cell types across Panel 4D, with particularly high expression seen in activated Th1 cells, activated Th2 cells, activated T regulatory cells, cytokine-activated and resting dermal and lung fibroblasts, and cytokine-activated endothelia from several sources. The NOV5 gene encodes a LRR/GPCR with predicted serine-threonine kinase activity and may therefore be a suitable target for small molecule drug discovery for the treatment of autoimmune and inflammatory diseases.

NOV6

Expression of gene NOV6 was assessed using the primer-probe set Ag1471, described in Table 39. Results from RTQ-PCR runs are shown in Table 40.

TABLE 39
Probe Name Ag1471

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-CCATCATCCATGAAGAAAAGG-3′ | 59.4 | 21 | 254 | 96 |
| Probe | TET-5′- | 69.9 | 26 | 304 | 97 |
| | AAGGGAGACCTGGCCTTCCTCAACTT-3′- | | | | |
| | TAMRA | | | | |
| Reverse | 5′GAGTCTGCTGCAGGTTGTTCT-3′ | 59.7 | 21 | 332 | 98 |

TABLE 40
Panel 1.2

| | Relative | | Relative |
|---|---|---|---|
| | Expression (%) | | Expression (%) |
| | 1.2tm1924t_ | | 1.2tm1924t_ |
| Tissue Name | ag1471 | Tissue Name | ag1471 |
| Endothelial cells | 15.9 | Renal Ca. 786-0 | 3.4 |
| Heart (fetal) | 63.7 | Renal Ca. A498 | 10.0 |
| Pancreas | 1.5 | Renal ca. RXF 393 | 22.1 |
| Pancreatic ca. CAPAN 2 | 2.1 | Renal ca. ACHN | 13.8 |
| Adrenal Gland (new lot*) | 74.7 | Renal Ca. UO-3 1 | 20.2 |
| Thyroid | 1.4 | Renal Ca. TK-10 | 19.3 |
| Salivary gland | 27.9 | Liver | 40.6 |
| Pituitary gland | 0.9 | Liver (fetal) | 22.1 |
| Brain (fetal) | 0.5 | Liver Ca. (hepatoblast) HepG2 | 4.0 |
| Brain (whole) | 1.8 | Lung | 9.4 |
| Brain (amygdala) | 3.7 | Lung (fetal) | 7.6 |
| Brain (cerebellum) | 1.5 | Lung ca. (small cell) LX-1 | 2.6 |
| Brain (hippocampus) | 10.3 | Lung ca. (small cell) NCI-H69 | 19.2 |
| Brain (thalamus) | 6.0 | Lung ca. (s.cell var.) SHP-77 | 2.0 |
| Cerebral Cortex | 21.8 | Lung ca. (large cell)NCI-H460 | 50.7 |
| Spinal cord | 2.4 | Lung ca. (non-sm. cell) A549 | 15.9 |
| CNS ca. (glio/astro) U87-MG | 37.4 | Lung ca. (non-s.cell) NCI-H23 | 20.3 |
| CNS ca. (glio/astro) U-118-MG | 20.0 | Lung ca (non-s.cell) HOP-62 | 55.9 |
| CNS ca. (astro) SW1783 | 8.7 | Lung ca. (non-s.d) NCI-H522 | 25.2 |
| CNS ca.* (neuro; met) SK-N- | 9.8 | Lung ca. (squam.) SW 900 | 43.5 |
| AS | | | |
| CNS ca. (astro) SF-539 | 2.3 | Lung ca. (squam.) NCI-H596 | 14.8 |
| CNS ca. (astro) SNB-75 | 2.6 | Mammary gland | 9.7 |
| | | Breast ca.* (p1. effusion) MCF- | 20.0 |
| CNS ca. (glio) SNB-19 | 1.8 | 7 | |
| | | Breast ca.* (pl.ef) MDA-MB- | 1.5 |
| CNS ca. (glio) U251 | 1.9 | 231 | |
| CNS ca. (glio) SF-295 | 37.1 | Breast ca.* (pl. effusion) T47D | 15.4 |
| Heart | 100.0 | Breast ca. BT-549 | 6.2 |
| Skeletal Muscle (new lot*) | 57.0 | Breast ca. MDA-N | 4.3 |
| Bone marrow | 6.2 | Ovary | 60.7 |
| Thymus | 1.4 | Ovarian ca. OVCAR-3 | 32.5 |
| Spleen | 15.3 | Ovarian ca. OVCAR-4 | 36.9 |
| Lymph node | 2.0 | Ovarian ca. OVCAR-5 | 16.7 |
| Colorectal | 8.8 | Ovarian ca. OVCAR-8 | 9.3 |
| Stomach | 3.8 | Ovarian ca. IGROV-1 | 12.9 |
| Small intestine | 20.6 | Ovarian ca.* (ascites) SK-OV-3 | 36.9 |
| Colon Ca. SW480 | 1.9 | Uterus | 5.8 |
| Colon ca.* (SW480 met)SW620 | 4.0 | Placenta | 5.2 |
| Colon ca. HT29 | 3.1 | Prostate | 33.7 |
| Colon Ca. HCT-116 | 5.4 | Prostate ca.* (bone met)PC-3 | 7.9 |
| Colon Ca. CaCo-2 | 3.1 | Testis | 0.7 |
| 83219 CC Well to Mod Diff | 7.2 | Melanoma Hs688(A).T | 2.2 |
| (ODO3866) | | | |
| Colon ca. HCC-2998 | 34.2 | Melanoma* (met) Hs688(B).T | 2.0 |
| Gastric ca.* (liver met) NCI- | 5.3 | | |
| N87 | | Melanoma UACC-62 | 4.8 |
| Bladder | 93.3 | Melanoma M14 | 2.3 |
| Trachea | 1.8 | Melanoma LOX IMVI | 5.2 |
| Kidney | 62.0 | Melanoma* (met) SK-MEL-5 | 2.7 |
| Kidney (fetal) | 7.8 | Adipose | 81.2 |

Panel 1.2 Summary:

Ag1471 Expression of the NOV6 gene is high to moderate in the majority of the samples on this panel. Highest expression is detected in heart (CT=22). Therefore, this gene may play a role in cardiovascular diseases including cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, and transplantation. In addition, the NOV6 gene is more highly expressed in adult kidney (CT=22.4) when compared to fetal kidney (CT=25.4). Thus, this gene may act in the differentiation of adult kidney cells and therapeutic modulation of the NOV6 gene product is of use in hyperproliferative diseases of the kidney, such as polycystic kidney disease.

The NOV6 gene encodes a protein that is highly homologous to nuclear factor kappa B inhibitor alpha, a protein that inhibits the proinflammatory transcription factor nuclear factor kappa B. Among metabolically relevant tissues, this gene has high expression in fetal and adult heart (CT=22), adrenal gland (CT=22), skeletal muscle (CT=22.5) and fetal and adult liver (CT=23-24). It also is moderately expressed in pancreas (CT=28), thyroid (CT=28) and pituitary gland (CT=28.5). Thus, the NOV6 gene product (or agonists of this protein) may be a drug treatment for the prevention and/or treatment of inflammatory conditions in each of the above tissues.

The NOV6 gene is also highly expressed in the brain in at least the thalamus, cerebral cortex, amygdala, cerebellum, hippocampus and thalamus, as well as the spinal cord. The close homology of this gene to the inhibitor of NF-kappaB (IkappaB) suggests that it possesses analogous function in the CNS. IkappaB is a critical mediator of neuronal apoptosis in a number of important pathological processes, including oxidative or nitrosative stress, hypoxia-ischaemia and excitoxicity. These processes are thought to underlie neuronal cell death at the heart of a number of diseases, including stroke, and neurodegenerative diseases such as Alzheimer's Disease, Parkinson's Disease, and trinucleotide repeat disorders, among others. Therefore, the NOV6 gene product and agents that modulate its action could act as therapeutic agents for the treatment of these disorders. Moreover, the role of NF-kappaB in synaptic processes underlying learning and memory suggest a possible utility for this gene product and agents that modulate its action in memory disorders. The role of NF-kappaB in inflammation also suggest a utility for the NOV6 gene product and agents that modulate its action in CNS disorders involving inflammation, such as neurodegenerative diseases such as Alzheimer's Disease, Parkinson's disease, Huntington's Disease and others.

NOV7

Expression of gene NOV7 was assessed using the primer-probe set Ag2440, described in Table 41. Results from RTQ-PCR runs are shown in Tables 42 and 43.

TABLE 41
Probe Name Ag244O

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-AACAGCCATGCAACCAAAC-3′ | 59.6 | 19 | 356 | 99 |
| Probe | FAM-5′- | 67.6 | 29 | 375 | 100 |
| | TGCAGCAAGCAACATACTGATATTTCTGA- | | | | |
| | 3′-TAMRA | | | | |
| Reverse | 5′-TTTCTTCCTGGCAAATTTCC-3′ | 59.1 | 20 | 414 | 101 |

TABLE 42
Panel 2D

| | Relative | | Relative |
|---|---|---|---|
| | Expression (%) | | Expression (%) |
| | 2Dtm3071f_ | | 2Dtm3071f_ |
| Tissue Name | ag2440 | Tissue Name | ag2440 |
| Normal Colon GENPAK | 34.2 | Kidney NAT Clontech 8120608 | 0.0 |
| 061003 | | | |
| 83219 CC Well to Mod Diff | 7.5 | Kidney Cancer Clontech | |
| (ODO3866) | | 8120613 | 3.1 |
| 83220CC NAT (ODO3866) | 5.7 | Kidney NAT Clontech 8120614 | 1.9 |
| 83221 CC Gr.2 rectosigmoid | 0.4 | Kidney Cancer Clontech | 0.0 |
| (ODO3868) | | 9010320 | |
| 83222 CC NAT (ODO3868) | 0.0 | Kidney NAT Clontech 9010321 | 0.6 |
| 83235 CC Mod Diff | 0.0 | Normal Uterus GENPAK | 0.0 |
| (ODO3920) | | 061018 | |
| | | Uterus Cancer GENPAK | 2.3 |
| 83236 CC NAT (ODO3920) | 4.6 | 064011 | |
| 83237 CC Gr.2 ascend colon | 6.2 | Normal Thyroid Clontech A + | 0.6 |
| (ODO3921) | | 6570-1 | |
| | | Thyroid Cancer GENPAK | 5.8 |
| 83238 CC NAT (ODO3921) | 3.7 | 064010 | |
| 83241 CC from Partial | 17.9 | Thyroid Cancer INVITROGEN | 4.8 |
| Hepatectomy (ODO4309) | | A302152 | |
| | | Thyroid NAT INVITROGEN | 3.9 |
| 83242 Liver NAT (ODO4309) | 2.7 | A302153 | |
| 87472 Colon mets to lung | 2.2 | Normal Breast GENPAK | 17.9 |
| (OD04451-01) | | 061019 | |
| 87473 Lung NAT (OD04451- | 1.2 | 84877 Breast Cancer | 0.0 |
| 02) | | (ODO4566) | |
| Normal Prostate Clontech A + | 0.0 | 85975 Breast Cancer | |
| 6546-1 | | (ODO4590-01) | 5.3 |
| 84140 Prostate Cancer | 0.0 | 85976 Breast Cancer Mets | |
| (OD04410) | | (ODO4590-03) | 3.9 |
| 84141 Prostate NAT | 0.0 | 87070 Breast Cancer Metastasis | |
| (OD04410) | | (ODO4655-05) | 35.4 |
| 87073 Prostate Cancer | 9.3 | GENPAK Breast Cancer | |
| (ODO4720-01) | | 064006 | 10.7 |
| 87074 Prostate NAT | 9.8 | | |
| (OD04720-02) | | Breast Cancer Res. Gen. 1024 | 34.9 |
| Normal Lung GENPAK 061010 | 18.6 | Breast Cancer Clontech | 0.0 |
| | | 9100266 | |
| 83239 Lung Met to Muscle | 0.7 | | |
| (ODO4286) | | Breast NAT Clontech 9100265 | 0.0 |
| 83240 Muscle NAT | 0.0 | Breast Cancer INVITROGEN | 40.6 |
| (ODO4286 | | A209073 | |
| 84136 Lung Malignant Cancer | 0.0 | Breast NAT INVITROGEN | 7.7 |
| (OD03126) | | A2090734 | |
| | | Normal Liver GENPAK | 0.0 |
| 84137 LungNAT (OD03126) | 0.0 | 061009 | |
| 84871 Lung Cancer (OD04404) | 0.0 | Liver Cancer GENPAK 064003 | 0.9 |
| | | Liver Cancer Research Genetics | 0.8 |
| 84872 Lung NAT (OD04404) | 0.0 | RNA 1025 | |
| | | Liver Cancer Research Genetics | 0.0 |
| 84875 Lung Cancer (OD04565) | 0.2 | RNA 1026 | |
| | | Paired Liver Cancer Tissue | 0.0 |
| | | Research Genetics RNA 6004- | |
| 84876 Lung NAT (OD04565) | 0.0 | T | |
| 85950 Lung Cancer (OD04237- | 0.5 | Paired Liver Tissue Research | 0.3 |
| 01) | | Genetics RINA 6004-N | |
| | | Paired Liver Cancer Tissue | 0.0 |
| 85970 Lung NAT (ODO4237- | 0.0 | Research Genetics RINA 6005- | |
| 02) | | T | |
| 83255 Ocular Mel Met to Liver | 10.7 | Paired Liver Tissue Research | 0.0 |
| (ODO4310) | | Genetics RNA 6005-N | |
| 83256 Liver NAT (ODO4310) | 0.0 | Normal Bladder GENPAK | 6.2 |
| | | 061001 | |
| 84139 Melanoma Mets to Lung | 2.0 | Bladder Cancer Research | 0.6 |
| (OD04321) | | Genetics RNA 1023 | |
| | | Bladder Cancer INVITROGEN | 0.0 |
| 84138 LungNAT (OD04321) | 2.1 | A302173 | |
| Normal Kidney GENPAK | 100.0 | 87071 Bladder Cancer | 0.0 |
| 061008 | | (OD04718-01) | |
| 83786 Kidney Ca, Nuclear | 2.1 | 87072 Bladder Normal | 0.0 |
| grade 2 (OD04338) | | Adjacent (ODO4718-03) | |
| 83787 Kidney NAT (OD04338) | 27.9 | Normal Ovary Res. Gen. | 0.0 |
| 83788 Kidney Ca Nuclear grade | 0.2 | Ovarian Cancer GENPAK | 4.9 |
| 1/2 (OD04339) | | 064008 | |
| | | 87492 Ovary Cancer | 0.6 |
| 83789 Kidney NAT (OD04339) | 0.8 | (OD04768-07 | |
| 83790 Kidney Ca. Clear cell | 0.0 | 87493 Ovary NAT (OD04768- | 0.0 |
| type (OD04340) | | 08) | |
| | | Normal Stomach GENPAK | 9.9 |
| 83791 Kidney NAT (OD04340) | 1.7 | 061017 | |
| 83792 Kidney Ca. Nuclear | 0.0 | Gastric Cancer Clontech | 0.0 |
| grade 3 OD04348 | | 9060358 | |
| | | NAT Stomach Clontech | 0.0 |
| 83793 Kidney NAT (OD04348) | 10.7 | 9060359 | |
| 87474 Kidney Cancer | 0.0 | Gastric Cancer Clontech | 1.2 |
| (OD04622-01) | | 9060395 | |
| 87475 Kidney NAT (OD04622- | 0.0 | NAT Stomach Clontech | 1.4 |
| 03) | | 9060394 | |
| 85973 Kidney Cancer | 0.0 | Gastric Cancer Clontech | 4.6 |
| (OD04450-01) | | 9060397 | |
| 85974 Kidney NAT (OD04450- | 0.0 | NAT Stomach Clontech | 0.0 |
| 03) | | 9060396 | |
| Kidney Cancer Clontech | 0.0 | Gastric Cancer GENPAK | 9.9 |
| 8120607 | | 064005 | |

TABLE 43
Panel 4D

| | Relative | | Relative |
|---|---|---|---|
| | Expression | | Expression |
| | (%) | | (%) |
| | 4Dtm3072f_{—} | | 4Dtm3072f_{—} |
| Tissue Name | ag2440 | Tissue Name | ag2440 |
| 93768_Secondary Th1_anti- | 0.0 | 93100_HUVEC | 0.0 |
| CD28/anti-CD3 | | (Endothelial)_IL-lb | |
| 93769_Secondary Th2_anti- | 0.0 | 93779_HUVEC | 21.0 |
| CD28/anti-CD3 | | (Endothelial)_IFN gamma | |
| | | 93102_HUVEC | 0.0 |
| 93770_Secondary Tr1_anti- | 0.0 | (Endothelial)_TNF alpha + IFN | |
| CD2S/anti-CD3 | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial) IL-11 | |
| 93571_Secondary Tr1_resting | 0.0 | 93583_Lung Microvascular | 0.0 |
| day 4-6 in IL-2 | | Endothelial Cells_none | |
| | | 93584_Lung Microvascular | 0.0 |
| 93568_primary Th1_anti- | 0.0 | Endothelial Cells_TNFa (4 | |
| CD28/anti-CD3 | | ng/ml) and IL1b (1 ng/ml) | |
| 93569_primary Th2_anti- | 0.0 | 92662_Microvascular Dermal | 0.0 |
| CD28/anti-CD3 | | endothelium_none | |
| | | 92663_Microsvasular Dermal | 0.0 |
| 93570_primary Tr1_anti- | 0.0 | endothelium_TNFa (4 ng/ml) | |
| CD28/anti-CD3 | | and IL1b (1 ng/ml) | |
| | | 93773_Bronchial | 1.7 |
| 93565_primary Th1_resting dy | 0.0 | epithelium_TNFa (4 ng/ml) and | |
| 4-6 in IL-2 | | IL1b (1 ng/ml)** | |
| 93566_primary Th2_resting dy | 0.0 | 93347_Small Airway | 0.0 |
| 4-6 in IL-2 | | Epithelium_none | |
| | | 93348_Small Airway | 0.0 |
| 93567_primary Tr1_resting dy | 0.0 | Epithelium_TNFa (4 ng/ml) | |
| 4-6 in IL-2 | | and IL1b (1 ng/ml) | |
| 93351_CD45RACD4 | 0.0 | | |
| lymphocyte_anti-CD28/anti- | | 92668_Coronery Artery | 0.0 |
| CD3 | | SMC_resting | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | | |
| CD28/anti-CD3 | | 93107_astrocytes resting | 0.0 |
| 93353_chronic CD8 | | | |
| Lymphocytes 2ry_resting dy 4- | 0.0 | 93108_astrocytes_TNFa (4 | 0.0 |
| 6 in IL-2 | | ng/ml) and IL1b (1 ng/ml) | |
| 93574_chronic CD8 | | | |
| Lymphocytes 2ry_activated | 0.0 | 92666_KU-812 | 0.0 |
| CD3/CD28 | | (Basophil)_resting | |
| | | 92667_KU-812 | 12.1 |
| 93354_CD4_none | 0.0 | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| | | 93580_CCD1106 | 0.0 |
| | | (Keratinocytes)_TNFa and | |
| 93103_LAK cells_resting | 4.8 | IFNg ** | |
| 93788_LAK cells_IL-2 | 4.7 | 93791_Liver Cirrhosis | 25.2 |
| 93787_LAK cells_IL-2 + IL-12 | 4.0 | 93792_Lupus Kidney | 20.6 |
| 93789_LAK cells_IL-2 + IFN | 4.1 | | |
| gamma | | 93577_NCI-H292 | 0.0 |
| 93790_LAK cells_IL-2 + IL-18 | 4.9 | 93358_NCI-H292_IL-4 | 0.0 |
| 93104_LAK | 0.0 | | |
| cells_PMA/ionomycin and IL- | | | |
| 18 | | 93360_NCI-H292_IL-9 | 0.0 |
| 93578_NK Cells IL-2_resting | 0.0 | 93359_NCI-H292_IL-13 | 0.0 |
| 93109_Mixed Lymphocyte | 6.2 | | |
| Reaction_Two Way MLR | | 93357_NCI-H292_IFN gamma | 0.0 |
| 93110_Mixed Lymphocyte | 4.9 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| | | 93253_Normal Human Lung | 0.0 |
| 93113_Mononuclear Cells | 9.2 | Fibroblast_TNFa (4 ng/ml) and | |
| (PBMCs)_PWM | | IL-1b (1 ng/ml) | |
| 93114_Mononuclear Cells | 0.0 | 93257_Normal Human Lung | 0.0 |
| (PBMCs)_PHA-L | | Fibroblast_IL-4 | |
| | | 93256_Normal Human Lung | 0.0 |
| 93249_Ramos (B cell)_none | 0.0 | Fibroblast_IL-9 | |
| 93250_Ramos(B | 0.0 | 93255_Normal Human Lung | 0.0 |
| cell)_ionomycin | | Fibroblast_IL-13 | |
| | | 93258_Normal Human Lung | 0.0 |
| 93349_B lymphocytes_PWM | 9.5 | Fibroblast_IFN gamma | |
| 93350_B lymphoytes_CD40L | 12.3 | 93106_Dermal Fibroblasts | 0.0 |
| and IL-4 | | CCD1070_resting | |
| 92665_EOL-1 | 0.0 | | |
| (Eosinophil)_dbcAMP | | 93361_Dermal Fibroblasts | 0.0 |
| differentiated | | CCD1070_TNF alpha 4 ng/ml | |
| 93248_EOL-1 | 0.0 | | |
| (Eosinophil)_dbcAMP/PMAion | | 93105_Dermal Fibroblasts | 0.0 |
| omycin | | CCD1070_IL-1 beta 1 ng/ml | |
| | | 93772_dermal fibroblast_IFN | 0.0 |
| 93356_Dendritic Cells none | 4.9 | gamma | |
| 93355_Dendritic Cells_LPS | 0.0 | | |
| 100 ng/ml | | 93771_dermal fibroblast_IL-4 | 0.0 |
| 93775_Dendritic Cells_anti- | 3.5 | | |
| CD40 | | 93259_IBD Colitis 1** | 8.8 |
| 93774_Monocytes_resting | 0.0 | 93260_IBD Colitis 2 | 3.3 |
| 93776_Monocytes_LPS 50 | 0.0 | | |
| ng/ml | | 93261_IBD Crohns | 2.9 |
| 93581_Macrophages_resting | 5.4 | 735010_Colon_normal | 57.0 |
| 93582_Macrophages_LPS 100 | 0.0 | | |
| ng/ml | | 735019_Lung_none | 12.3 |
| 93098_HUVEC | 0.0 | | |
| (Endothelial)_none | | 64028-1_Thymus_none | 100.0 |
| 93099_HUVEC | 0.0 | | |
| (Endothelial)_starved | | 64030-1_Kidney_none | 9.7 |

Panel 1.3D Summary:

Ag2440 Expression of the NOV7 gene is low/undetectable (CT values>35) across all of the samples on this panel (data not shown).

Panel 2D Summary:

Ag2440 The expression of the NOV7 gene is highest in normal kidney tissue (CT=30.8) and also shows low but significant expression in colon tissue and breast tissue. Of particular interest, is the higher expression of this gene observed in samples derived from breast cancers when compared to normal breast tissues. Thus, expression of the NOV7 gene could be used to distinguish breast cancer cells from normal breast tissue. In addition, therapeutic modulation of protein encoded by the NOV7 gene, through the use of small molecule drugs or antibodies, could be of utility in the treatment of breast cancer.

Panel 4D Summary:

Ag2440 Expression of the NOV7 gene is highest in the thymus, but nevertheless is very moderate (CT 33.1). Therefore, protein therapeutics or antibodies against the gene product encoded by the NOV7 gene could be of use in T cell mediated disease and autoimmunity. This gene is also expressed at low levels in colon (CT=33.9).

Panel CNS_neurodegeneration_v1.0 Summary:

Ag2440 Expression of the NOV7 gene is low/undetectable (CT values>35) across all of the samples on this panel (data not shown).

NOV8

Expression of gene NOV8 was assessed using the primer-probe sets Ag1507, Ag1558, and Ag1602 (identical sequences), described in Table 44. Results from RTQ-PCR runs are shown in Tables 45, 46, and 47.

TABLE 44
Probe Name Ag15O7/Ag1558/Ag16O2

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-CCCCTGATTTACACAGCTTTTA- | 58.3 | 22 | 1076 | 102 |
| | 3′ | | | | |
| Probe | TET-5′- | 66.4 | 26 | 1107 | 103 |
| | ACAACAATGCCTTCAAGAGCCTCTTT- | | | | |
| | 3′-TAMRA | | | | |
| Reverse | 5′-CCCTGTGTTCATCTCTGCTTAG- | 59 | 22 | 1134 | 104 |
| | 3′ | | | | |

TABLE 45
Panel 1.2

| | Relative | | Relative |
|---|---|---|---|
| | Expression (%) | | Expression (%) |
| | 1.2tm2155t_ | | 1.2tm2155t_ |
| Tissue Name | ag1507 | Tissue Name | ag1507 |
| Endothelial cells | 0.3 | Renal Ca. 786-0 | 0.0 |
| Heart (fetal) | 0.2 | Renal ca. A498 | 1.1 |
| Pancreas | 0.3 | Renal ca. RXF 393 | 0.0 |
| Pancreatic Ca. CAPAN 2 | 0.1 | Renal ca. ACHN | 0.6 |
| Adrenal Gland (new lot*) | 0.2 | Renal Ca. UO-31 | 0.7 |
| Thyroid | 0.0 | Renal ca. TK-10 | 1.5 |
| Salivary gland | 0.5 | Liver | 0.2 |
| Pituitary gland | 0.0 | Liver (fetal) | 0.0 |
| Brain (fetal) | 0.0 | Liver ca. (hepatoblast) HepG2 | 1.1 |
| Brain (whole) | 0.2 | Lung | 0.0 |
| Brain (amygdala) | 0.8 | Lung (fetal) | 0.0 |
| Brain (cerebellum) | 0.1 | Lung ca. (small cell) LX-1 | 0.3 |
| Brain (hippocampus) | 0.5 | Lung Ca. (small cell) NCI-H69 | 1.3 |
| Brain (thalamus) | 0.1 | Lung Ca. (s.cell var.) SHP-77 | 0.0 |
| Cerebral Cortex | 0.6 | Lung Ca. (large cell)NCI-H460 | 0.2 |
| Spinal cord | 0.0 | Lung Ca. (non-sm. cell) A549 | 0.8 |
| CNS Ca. (glio/astro) U87-MG | 0.4 | Lung Ca. (non-s.cell) NCI-H23 | 1.0 |
| CNS Ca. (glio/astro) U-118-MG | 0.1 | Lung Ca (non-s.cell) HOP-62 | 1.4 |
| CNS Ca. (astro) SW1783 | 0.0 | Lung ca. (non-s.d) NCI-H522 | 0.8 |
| CNS Ca.* (neuro; met) SK-N- | | | |
| AS | 0.0 | Lung Ca. (squam.) SW 900 | 0.8 |
| CNS Ca. (astro) SF-539 | 0.2 | Lung ca. (squam.) NCI-H596 | 0.1 |
| CNS Ca. (astro) SNB-75 | 0.0 | Mammary gland | 0.0 |
| | | Breast ca.* (pl. effusion) MCF- | |
| CNS ca. (glio) SNB-19 | 0.6 | 7 | 0.0 |
| | | Breast Ca* (pl.ef) MDA-MB- | |
| CNS Ca. (gito) U251 | 0.4 | 231 | 0.1 |
| CNS Ca. (glio) SF-295 | 0.1 | Breast Ca.* (pl. effusion) T47D | 0.8 |
| Heart | 0.7 | Breast ca. BT-549 | 0.4 |
| Skeletal Muscle (new lot*) | 0.0 | Breast Ca. MDA-N | 1.2 |
| Bone marrow | 0.0 | Ovary | 0.7 |
| Thymus | 0.0 | Ovarian Ca. OVCAR-3 | 0.2 |
| Spleen | 0.2 | Ovarian Ca. OVCAR-4 | 0.5 |
| Lymph node | 0.0 | Ovarian Ca. OVCAR-5 | 3.9 |
| Colorectal | 0.2 | Ovarian Ca. OVCAR-8 | 2.8 |
| Stomach | 0.0 | Ovarian Ca. IGROV-1 | 1.9 |
| Small intestine | 0.2 | Ovarian ca.* (ascites) SK-OV-3 | 1.4 |
| Colon ca. SW480 | 0.0 | Uterus | 0.0 |
| Colon Ca.* (SW480 met)SW620 | 0.0 | Placenta | 0.0 |
| Colon Ca. HT29 | 0.6 | Prostate | 0.1 |
| Colon Ca. HCT-116 | 0.5 | Prostate Ca* (bone met)PC-3 | 0.6 |
| Colon Ca. CaCo-2 | 0.1 | Testis | 1.2 |
| 83219 CC Well to Mod Diff | | | |
| (ODO3866) | 0.7 | Melanoma Hs688(A).T | 0.0 |
| Colon Ca. HCC-2998 | 1.4 | Melanoma* (met) Hs688(B).T 0.3 | |
| Gastric ca.* (liver met) NCI- | | | |
| N87 | 0.6 | Melanoma UACC-62 | 0.2 |
| Bladder | 1.5 | Melanoma M14 | 2.2 |
| Trachea | 0.0 | Melanoma LOX IMVI | 0.5 |
| Kidney | 1.1 | Melanoma* (met) SK-MEL-5 | 0.1 |
| Kidney (fetal) | 0.3 | Adipose | 100.0 |

TABLE 46
Panel 2D

| | Relative | | Relative |
|---|---|---|---|
| | Expression (%) | | Expression (%) |
| | 2dtm4625t_ | | 2dtm4625t_ |
| Tissue Name | ag1602 | Tissue Name | ag1602 |
| Normal Colon GENPAK | 35.6 | | |
| 061003 | | Kidney NAT Clontech 8120608 | 0.0 |
| 83219 CC Well to Mod Diff | 47.3 | Kidney Cancer Clontech | 3.8 |
| ODO3866 | | 8120613 | |
| 83220 CC NAT (ODO3866) | 11.3 | Kidney NAT Clontech 8120614 | 0.0 |
| 83221 CC Gr.2 rectosigmoid | 27.2 | Kidney Cancer Clontech | 14.2 |
| (ODO3868) | | 9010320 | |
| 83222 CC NAT (ODO3868) | 4.0 | Kidney NAT Clontech 9010321 | 18.3 |
| 83235 CC Mod Diff | 0.0 | Normal Uterus GENPAK | 0.0 |
| (ODO3920) | | 061018 | |
| | | Uterus Cancer GENPAK | 18.2 |
| 83236 CC NAT (ODO3920) | 9.0 | 064011 | |
| 83237 CC Gr.2 ascend colon | 0.0 | Normal Thyroid Clontech A + | 0.0 |
| (ODO3921) | | 6570-1 | |
| | | Thyroid Cancer GENPAK | 0.0 |
| 83238 CCNAT (ODO3921) | 27.9 | 064010 | |
| 83241 CC from Partial | 8.1 | Thyroid Cancer INVITROGEN | 5.0 |
| Hepatectomy (ODO4309) | | A302152 | |
| | | Thyroid NAT INVITROGEN | 18.7 |
| 83242 Liver NAT (ODO4309) | 8.7 | A302153 | |
| 87472 Colon mets to lung | 9.0 | Normal Breast GENPAK | 0.0 |
| (OD04451-01) | | 061019 | |
| 87473 Lung NAT (OD04451- | 15.5 | 84877 Breast Cancer | 31.0 |
| 02) | | (ODO4566) | |
| Normal Prostate Clontech A + | 22.7 | 85975 Breast Cancer | 7.7 |
| 6546-1 | | (ODO4590-01) | |
| 84140 Prostate Cancer | 0.0 | 85976 Breast Cancer Mets | 10.9 |
| (OD04410) | | (ODO4590-03) | |
| 84141 Prostate NAT | 10.8 | 87070 Breast Cancer Metastasis | 40.9 |
| (OD04410) | | (ODO4655-05) | |
| 87073 Prostate Cancer | 25.9 | GENPAK Breast Cancer | 8.5 |
| (OD04720-01) | | 064006 | |
| 87074 Prostate NAT | 25.7 | | |
| (OD04720-02) | | Breast Cancer Res. Gen. 1024 | 0.0 |
| | | Breast Cancer Clontech | 0.0 |
| Normal Lung GENPAK 061010 | 100.0 | 9100266 | |
| 83239 Lung Met to Muscle | 27.2 | | |
| (ODO4286) | | Breast NAT Clontech 9100265 | 0.0 |
| 83240 Muscle NAT | 28.5 | Breast Cancer INVITROGEN | 9.0 |
| (OD04286) | | A209073 | |
| 84136 Lung Malignant Cancer | 11.5 | Breast NAT INVITROGEN | 25.9 |
| OD03126 | | A2090734 | |
| | | Normal Liver GENPAK | 17.3 |
| 84137 Lung NAT(OD03126) | 11.2 | 061009 | |
| 84871 Lung Cancer (OD04404) | 10.1 | Liver Cancer GENPAK 064003 | 13.6 |
| | | Liver Cancer Research Genetics | 10.2 |
| 84872 Lung NAT (OD04404) | 0.0 | RNA 1025 | |
| | | Liver Cancer Research Genetics | 0.0 |
| 84875 Lung Cancer (OD04565) | 0.0 | RNA 1026 | |
| | | Paired Liver Cancer Tissue | 9.3 |
| | | Research Genetics RNA 6004- | |
| 84876 Lung NAT (OD04565) | 7.4 | T | |
| 85950 Lung Cancer (OD04237- | 0.0 | Paired Liver Tissue Research | 0.0 |
| 01) | | Genetics RNA 6004-N | |
| | | Paired Liver Cancer Tissue | |
| 85970 Lung NAT (OD04237- | 17.7 | Research Genetics RNA 6005- | 10.0 |
| 02) | | T | |
| 83255 Ocular Mel Met to Liver | 0.0 | Paired Liver Tissue Research | 0.0 |
| (ODO4310) | | Genetics RNA 6005-N | |
| | | Normal Bladder GENPAK | 0.0 |
| 83256 Liver NAT (ODO4310) | 0.0 | 061001 | |
| 84139 Melanoma Mets to Lung | 0.0 | Bladder Cancer Research | 0.0 |
| (OD04321) | | Genetics RNA 1023 | |
| | | Bladder Cancer INVITROGEN | 32.1 |
| 84138 Lung NAT (OD04321) | 27.4 | A302173 | |
| Normal Kidney GENPAK | 9.5 | 87071 Bladder Cancer | 9.3 |
| 061008 | | (ODO4718-01 | |
| 83786 Kidney Ca, Nuclear | 0.0 | 87072 Bladder Normal | 6.3 |
| grade 2 (OD04338) | | Adjacent (ODO4718-03) | |
| 83787 Kidney NAT (OD04338) | 0.0 | Normal Ovary Res. Gen. | 8.5 |
| 83788 Kidney Ca Nuclear grade | 27.5 | Ovarian Cancer GENPAK | 10.2 |
| 1/2 OD04339 | | 064008 | |
| | | 87492 Ovary Cancer | 27.0 |
| 83789 Kidney NAT (OD04339) | 28.5 | OD04768-07 | |
| 83790 Kidney Ca, Clear cell | 16.0 | 87493 Ovary NAT (ODO4768- | 0.0 |
| type (OD04340) | | 08) | |
| | | Normal Stomach GENPAK | 5.0 |
| 83791 Kidney NAT (OD04340) | 17.9 | 061017 | |
| 83792 Kidney Ca, Nuclear | 0.0 | Gastric Cancer Clontech | 0.0 |
| grade 3 (OD04348) | | 9060358 | |
| | | NAT Stomach Clontech | 0.0 |
| 83793 Kidney NAT (OD04348) | 9.0 | 9060359 | |
| 87474 Kidney Cancer | 0.0 | Gastric Cancer Clontech | 3.9 |
| OD04622-01 | | 9060395 | |
| 87475 Kidney NAT (OD04622- | 0.0 | NAT Stomach Clontech | 18.2 |
| 03) | | 9060394 | |
| 85973 Kidney Cancer | 14.0 | Gastric Cancer Clontech | 9.9 |
| (OD04450-01 | | 9060397 | |
| 85974 Kidney NAT (OD04450- | 0.0 | NAT Stomach Clontech | 0.0 |
| 03) | | 9060396 | |
| Kidney Cancer Clontech | 0.0 | Gastric Cancer GENPAK | 50.7 |
| 8120607 | | 064005 | |

TABLE 47
Panel 4D

| | Relative | Relative |
|---|---|---|
| | Expression (%) | Expression (%) |
| | 4dx4tm5019t_{—} | 4dtm4117t_{—} |
| Tissue Name | ag1507_b1 | ag1558 |
| 93768_Secondary Th1_anti-CD28/anti-CD3 | 48.8 | 29.5 |
| 93769_Secondary Th2_anti-CD28/anti-CD3 | 17.4 | 31.9 |
| 93770_Secondary Tr1_anti-CD28/anti-CD3 | 10.7 | 18.0 |
| 93573_Secondary ml resting day 4-6 in IL-2 | 0.0 | 0.0 |
| 93572_Secondary Th2_resting day 4-6 in IL-2 | 8.3 | 7.5 |
| 93571_Secondary Tr1_resting day 4-6 in IL-2 | 0.0 | 7.3 |
| 93568_primary Th1_anti-CD28/anti-CD3 | 57.6 | 17.7 |
| 93569_primary Th2_anti-CD28/anti-CD3 | 8.0 | 42.0 |
| 93570_primary Tr1_anti -CD28/anti-CD3 | 27.2 | 43.2 |
| 93S65_primary Th1_resting dy 4-6 in IL-2 | 56.1 | 34.6 |
| 93s60_primary Th2_resting dy 4-6 in IL-2 | 23.2 | 20.0 |
| 93567_primary Tr1_resting dy 4-6 in IL-2 | 9.0 | 15.8 |
| 93351_CD4SRA CD4 lymphocyte_anti-CD28/anti-CD3 | 7.1 | 48.3 |
| 93352_CD45RO CD4 lymphocyte_anti-CD28/anti-CD3 | 34.5 | 31.0 |
| 93251_CD8 Lymphocytes_anti-CD28/anti-CD3 | 17.3 | 16.3 |
| 93353_chronic CD8 Lymphocytes 2ry_resting dy 4-6 in IL-2 | 8.3 | 32.5 |
| 93574_chronic CD8 Lymphocytes 2ry_activated CD3/CD28 | 10.4 | 12.3 |
| 93354_CD4_none | 13.9 | 15.8 |
| 93252_Secondary Th1/Th2/Tr1_anti-CD95 CH11 | 15.6 | 0.0 |
| 93103_LAK cells_resting | 17.1 | 54.7 |
| 93788_LAK cells_IL-2 | 30.5 | 13.4 |
| 93787_LAK cells_IL-2 + IL-12 | 25.1 | 8.0 |
| 93789_LAK cells_IL-2 + IFN gamma | 51.0 | 30.4 |
| 93790_LAK cells_lL-2 + IL-18 | 12.4 | 84.1 |
| 93104_LAK cells_PMA/ionomycin and IL-18 | 16.7 | 24.8 |
| 93578_NK Cells IL-2_resting | 37.0 | 32.3 |
| 93109_Mixed Lymphocyte Reaction_Two Way MLR | 8.1 | 48.6 |
| 93110_Mixed Lymphocyte Reaction_Two Way MLR | 7.5 | 15.7 |
| 93111_Mixed Lymphocyte Reaction_Two Way MLR | 7.4 | 0.0 |
| 93112_Mononuclear Cells (PBMCs)_resting | 0.0 | 7.2 |
| 93113_Mononuclear Cells (PBMCs)_PWM | 100.0 | 64.2 |
| 93114_Mononuclear Cells (PBMCs)_PHA-L | 71.0 | 23.8 |
| 93249_Ramos (B cell)_none | 0.0 | 8.1 |
| 93250_Ramos (B cell)_ionomycin | 42.1 | 36.9 |
| 93349_B lymphocytes_PWM | 12.7 | 69.3 |
| 93350_B lymphoytes_CD40L and IL-4 | 45.9 | 45.1 |
| 92665_EOL-1 (Eosinophil)_dbcAMP differentiated | 9.1 | 3.2 |
| 93248_EOL-1 (Bosinophil)_dbcAMP/PMAionomycin | 6.6 | 30.4 |
| 93356_Dendritic Cells_none | 51.8 | 26.8 |
| 93355_Dendritic Cells_LPS 100 ng/ml | 15.3 | 0.0 |
| 93775_Dendritic Cells_anti-CD40 | 20.8 | 0.0 |
| 93774_Monocytes resting | 7.4 | 0.0 |
| 93776_Monocytes_LPS 50 ng/ml | 47.8 | 37.1 |
| 93581_Macrophages_resting | 22.2 | 32.3 |
| 93582_Macrophages_LPS 100 ng/ml | 0.0 | 16.3 |
| 93098_HUVEC (Endothelial)_none | 0.0 | 0.0 |
| 93099_HUVEC (Endothelial)_starved | 10.9 | 30.6 |
| 93100_HUVEC (Endothelial)_IL-1b | 0.0 | 0.0 |
| 93779_HUVEC (Endothelial) IFN gamma | 0.0 | 8.5 |
| 93102_HUVEC (Endothelial)_TNF alpha + IFN gamma | 0.0 | 18.2 |
| 93101_HUVEC (Endothelial)_TNF alpha + IL4 | 0.0 | 0.0 |
| 93781_HUVEC (Endothelial)_IL-11 | 0.0 | 0.0 |
| 93583_Lung Microvascular Endothelial Cells_none | 5.1 | 4.2 |
| 93584_Lung Microvascular Endothelial Cells_TNFa (4 ng/ml) | 0.0 | 7.6 |
| and IL1b (1 ng/ml) | | |
| 92662_Microvascular Dermal endothelium_none | 19.2 | 6.4 |
| 92663_Microsvasular Dermal endothelium_TNFa (4 ng/ml) and | 9.6 | 0.0 |
| IL1b(1 ng/ml) | | |
| 93773_Bronchial epithelium_TNFa (4 ng/ml) and IL1b (1 | 0.0 | 0.0 |
| ng/ml)** | | |
| 93347_Small Airway Epithelium_none | 0.0 | 7.6 |
| 93348_Small Airway Epithelium_TNFa (4 ng/ml) and IL1b (1 | 80.6 | 49.7 |
| ng/ml) | | |
| 92668_Coronery Artery SMC_resting | 10.3 | 0.0 |
| 92669_Coronery Artery SMC_TNFa (4 ng/ml) and IL1b (1 | 7.3 | 7.9 |
| ng/ml) | | |
| 93107_astrocytes resting | 0.0 | 0.0 |
| 93108_astrocytes_TNFa (4 ng/ml) and IL1b (1 ng/ml) | 0.0 | 8.2 |
| 92666_KU-812 (Basophil)_resting | 0.0 | 7.6 |
| 92667_KU-812 (Basophil)_PMA/ionoycin | 20.9 | 7.3 |
| 93579_CCD1106 (Keratinocytes)_none | 4.2 | 7.3 |
| 93580_CCD1106 (Keratinocytes)_TNFa and IFNg** | 0.0 | 0.0 |
| 93791_Liver Cirrhosis | 18.8 | 94.6 |
| 93792_Lupus Kidney | 0.0 | 0.0 |
| 93577_NCI-H292 | 14.5 | 14.6 |
| 93358_NCI-H292 IL-4 | 16.4 | 23.5 |
| 93360_NCI-H292_IL-9 | 28.0 | 7.3 |
| 93359_NCI-H292_IL-13 | 18.9 | 23.0 |
| 93357_NCI-H292_IFN gamma | 13.3 | 8.0 |
| 93777_HPAEC - | 0.0 | 10.4 |
| 93778_HPAEC_IL-1 beta/TNA alpha | 18.9 | 0.0 |
| 93254_Normal Human Lung Fibroblast_none | 0.0 | 0.0 |
| 93253_Normal Human Lung Fibroblast_TNFa (4 ng/ml) and IL- | 8.0 | 0.0 |
| 1b (1 ng/ml) | | |
| 93257_Normal Human Lung Fibroblast_IL-4 | 8.9 | 7.8 |
| 93256_Normal Human Lung Fibroblast_IL-9 | 7.7 | 16.3 |
| 93255_Normal Human Lung Fibroblast_IL-13 | 15.2 | 0.0 |
| 93258_Normal Human Lung Fibroblast_IFN gamma | 10.4 | 7.4 |
| 93106_Dermal Fibroblasts CCD1070_resting | 0.0 | 26.1 |
| 93361_Dermal Fibroblasts CCD1070_TNF alpha 4 ng/ml | 65.6 | 109.0 |
| 93105_Dermal Fibroblasts CCD1070 IL-1 beta 1 ng/ml | 14.7 | 31.0 |
| 93772_dermal fibroblast_IFN gamma | 0.0 | 9.6 |
| 93771_dermal fibroblast_IL-4 | 39.8 | 0.0 |
| 93259_IBD Colitis 1** | 6.6 | 0.0 |
| 93260_IBD Colitis 2 | 8.0 | 8.1 |
| 93261_IBD Crohns | 8.2 | 14.7 |
| 735010_Colon_normal | 30.5 | 48.3 |
| 735019_Lung_none | 14.5 | 11.7 |
| 64028-1_Thymus_none | 22.1 | 10.1 |
| 64030-1_Kidney_none | 0.0 | 0.0 |

Panel 1.2 Summary:

Ag1507 Expression of the NOV8 gene appears to be highest in adipose tissue. However, this sample is contaminated by genomic DNA and must therefore be disregarded. Taking this into account this gene is most highly expressed in a sample derived from an ovarian cancer cell line (OVCAR-5) (CT=32.5). Overall, there is a predominant pattern showing overexpression of the NOV8 gene in cancer cell lines, when compared to normal tissues. For example, relative overexpression of this gene is seen in ovarian cancer cell lines, melanoma cell lines, lung cancer cell lines, renal cancer cell lines and colon cancer cell lines. Thus, expression of the NOV8 gene could be used to distinguish cultured cell lines from normal tissues. In addition, these data indicate that the expression of this gene is associated with cancer and thus, therapeutic modulation of the NOV8 gene product is of use in the treatment of a variety of cancers.

Panel 1.3D Summary:

Ag1507/Ag1558/Ag1602 Expression of the NOV8 gene is low/undetectable (CT values>35) across all of the samples on this panel (data not shown).

Panel 2D Summary:

Ag1602 Significant expression of the NOV8 gene is limited to a sample of normal lung (CT=34.2). Therefore, NOV8 nucleic acids can be used as a marker to identify lung tissue. In addition, the NOV8 gene product may play a role in the development of lung diseases including asthma and emphysema. Ag1507/Ag1558 Expression of the NOV8 gene is low/undetectable (CT values>34.5) across all of the samples on this panel (data not shown).

Panel 4D Summary:

Ag1507/Ag1558 Expression of the NOV8 gene is low but significant in activated dermal fibroblasts and PHA stimulated PBMC (CT 34.4). Results from the experiment using Ag1507 are quite similar to Ag1558 except that expression is also seen in activated small airway epithelium (CT 34.6). This result is consistent with what was observed in Panel 2D. Expression in small airway epithelium is expected since the NOV8 gene encodes a protein with homology to the serotonin receptor. Therefore, the use of antibodies or the extracellular domain of this receptor could be beneficial for the treatment of allergic diseases such as asthma, eczema, atopic dermatitis, and any disease associated with delayed type hypersensitivity. Ag1602 Expression of the NOV8 gene is low/undetectable (CT values>34.5) across all of the samples on this panel (data not shown).

Panel CNSD.01 Summary:

Ag1602 Expression of the NOV8 gene is low/undetectable (CT values>34.5) across all of the samples on this panel (data not shown).

Panel CNS_neurodegeneration_v1.0 Summary:

Ag1507/Ag1558/Ag1602 Expression of the NOV8 gene is low/undetectable (CT values>34.5) across all of the samples on this panel (data not shown).

### Example 2.

SNP analysis of NOVX clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

Results

Variants are reported individually but any combination of all or a select subset of variants are also included as contemplated NOVX embodiments of the invention.

NOV1a SNP data:

NOV1a (clone sggc_draft_dj881p19_{—}20000725) has seven SNP variants, whose variant positions for its nucleotide and amino acid sequences is numbered according to SEQ ID NOS:1 and 2, respectively. The nucleotide sequence of the NOV1 variant differs as shown in Table 48.

TABLE 48
SNP and Coding Variants for NOV1a

| NT Position | Wild Type | | Amino Acid | Amino Acid |
|---|---|---|---|---|
| of cSNP | NT | Variant NT | position | Change |
| 61 | G | A | 17 | A->T |
| 280 | C | T | 88 | No change |
| 685 | T | C | 224 | F->L |
| 874 | A | G | 286 | T->A |
| 882 | C | T | 289 | No change |
| 896 | A | G | 294 | D->G |
| 943 | G | A | 309 | No change |

Further, NOV1a (X56842_da1) has seven SNP variants, whose variant positions for its nucleotide and amino acid sequences is numbered according to SEQ ID NOS:1 and 2, respectively. The nucleotide sequence of the NOV1 variant differs as shown in Table 49.

TABLE 49
SNP and Coding Variants for NOV1a

| NT Position of | | | |
|---|---|---|---|
| cSNP | Wild Type NT | Variant NT | Depth |
| 149 | C | T | 20 |
| 195 | T | C | 20 |
| 217 | T | C | 20 |
| 826 | G | A | 16 |

NOV1b SNP data:

NOV1b has seven SNP variants, whose variant positions for its nucleotide and amino acid sequences is numbered according to SEQ ID NOS:3 and 4, respectively. The nucleotide sequence of the NOV1b variant differs as shown in Table 50.

TABLE 50
SNP and Coding Variants for NOV1b

| NT Position | Wild Type | | Amino Acid | Amino Acid |
|---|---|---|---|---|
| of cSNP | NT | Variant NT | position | Change |
| 294 | C | T | 88 | No change |
| 700 | T | C | 234 | F->L |
| 889 | A | G | 287 | No change |
| 911 | A | G | 294 | D->G |
| 957 | G | A | 309 | No change |
| 993 | G | A | 321 | No change |

NOV3a SNP data:

NOV3a has seven SNP variants, whose variant positions for its nucleotide and amino acid sequences is numbered according to SEQ ID NOS:13 and 14, respectively. The nucleotide sequence of the NOV3a variant differs as shown in Table 51.

TABLE 51
SNP and Coding Variants for NOV3a

| NT Position | Wild Type | | Amino Acid | Amino Acid |
|---|---|---|---|---|
| of cSNP | NT | Variant NT | position | Change |
| 446 | T | C | 149 | F->L |
| 553 | A | G | 184 | No change |

NOV4a SNP data:

In the following positions, one or more consensus positions (Cons. Pos.) of the nucleotide sequence have been identified as SNPs. “Depth” rerepresents the number of clones covering the region of the SNP. The Putative Allele Frequency (Putative Allele Freq.) is the fraction of all the clones containing the SNP. A dash (“−”), when shown, means that a base is not present. The sign “>” means “is changed to”.

NOV4a has one SNP variant, whose variant positions for its nucleotide and amino acid sequences is numbered according to SEQ ID NOS:17 and 18, respectively. The nucleotide sequence of the NOV3a variant differs as shown in Table 52.

TABLE 52
cSNP and Coding Variants for NOV4a

| NT Position | Wild Type | | Amino Acid | Amino Acid |
|---|---|---|---|---|
| of cSNP | NT | Variant NT | position | Change |
| 471 | A | G | 129 | N->S |

NOV4b SNP data:

NOV4b has four SNP variants, whose variant positions for its nucleotide and amino acid sequences is numbered according to SEQ ID NOS:19 and 20, respectively. The nucleotide sequence of the NOV4b variant differs as shown in Table 53.

TABLE 53
cSNP and Coding Variants for NOV4b

| NT Position | Wild Type | | Amino Acid | Amino Acid |
|---|---|---|---|---|
| of cSNP | NT | Variant NT | position | Change |
| 183 | C | T | | None |
| 423 | G | A | 63 | D->N |
| 625 | A | G | 130 | N->S |

NOV6 SNP data:

NOV6 has three SNP variants, whose variant positions for its nucleotide and amino acid sequences is numbered according to SEQ ID NOS:25 and 26, respectively. The nucleotide sequence of the NOV6 variant differs as shown in Table 54.

TABLE 54
SNP and Coding Variants for NOV6

| NT Position | Wild Type | | Amino Acid | Amino Acid |
|---|---|---|---|---|
| of cSNP | NT | Variant NT | position | Change |
| 609 | G | A | 203 | No change |

NOV9 SNP data:

In the following positions, one or more consensus positions (Cons. Pos.) of the nucleotide sequence have been identified as SNPs. “Depth” rerepresents the number of clones covering the region of the SNP. The Putative Allele Frequency (Putative Allele Freq.) is the fraction of all the clones containing the SNP. A dash (“−”), when shown, means that a base is not present. The sign “>” means “is changed to.”

NOV9 has six SNP variants, whose variant positions for its nucleotide and amino acid sequences is numbered according to SEQ ID NOS:31 and 32, respectively. The nucleotide sequence of the NOV6 variant differs as shown in Table 55.

TABLE 55
SNP and Coding Variants for NOV6

| NT Position | Wild Type | | Amino Acid | Amino Acid |
|---|---|---|---|---|
| of cSNP | NT | Variant NT | position | Change |
| 116 | T | C | 5 | S->P |
| 131 | T | C | 10 | S->P |
| 142 | C | T | 13 | S->L |
| 196 | A | G | 31 | K->R |
| 267 | C | T | 55 | A->V |
| 281 | T | C | 60 | L->P |

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, and 32;
(b) a variant of a mature form of an amino acid sequence selected from the group consisting of SEQ ID NOS:2, 4, 6, 8, 10, 12, 14, 16, 18, 20, 22, 24, 26, 28, 30, and 32, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18, 20, 22, 24, 26, 28, 30, and 32; and
(d) a variant of an amino acid sequence selected from the group consisting of SEQ ID NOS:2, 4, 6, 8, 10, 12, 14, 16, 18, 20, 22, 24, 26, 28, 30, and 32; wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 16, 18, 20, 22, 24, 26, 28, 30, and 32.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 29, and 31.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, and 32;
(b) a variant of a mature form of an amino acid sequence selected from the group consisting of SEQ ID NOS:2, 4, 6, 8, 10, 12, 14, 16, 18, 20, 22, 24, 26, 28, 30, and 32, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, and 32;
(d) a variant of an amino acid sequence selected from the group consisting SEQ ID NOS:2, 4, 6, 8, 10, 12, 14, 16, 18, 20, 22, 24, 26, 28, 30, and 32, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, and 32, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, and 31.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, 21, 23, 25, 27, 29,and31;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, and 31, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS:1, 3, 5, 7, 9, 11, 13, 15, 17, 19, 21, 23, 25, 27, 29, and 31, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26, wherein said subject is a human.

28. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

29. The method of claim 28, wherein said subject is a human.

30. A method of treating or preventing a NOVX-associated disorder, said method comprising administering to a subject in which such treatment or prevention is desired the antibody of claim 15 in an amount sufficient to treat or prevent said NOVX-associated disorder in said subject.

31. The method of claim 30, wherein the subject is a human.

32. A pharmaceutical composition comprising the polypeptide of claim 1 and a pharmaceutically-acceptable carrier.

33. A pharmaceutical composition comprising the nucleic acid molecule of claim 5 and a pharmaceutically-acceptable carrier.

34. A pharmaceutical composition comprising the antibody of claim 15 and a pharmaceutically-acceptable carrier.

35. A kit comprising in one or more containers, the pharmaceutical composition of claim 32.

36. A kit comprising in one or more containers, the pharmaceutical composition of claim 33.

37. A kit comprising in one or more containers, the pharmaceutical composition of claim 34.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

39. The method of claim 38 wherein the predisposition is to cancers.

40. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

41. The method of claim 40 wherein the predisposition is to a cancer.

42. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, and 32, or a biologically active fragment thereof.

43. A method of treating a pathological state in a mammal, the method comprising administering to the mammal the antibody of claim 15 in an amount sufficient to alleviate the pathological state.
